SEQUENCE LISTING (137 sequences)

SEQ ID NO: 1 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 2 - protein, 153 aa, Homo sapiens
APVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 3 - protein, 330 aa, Homo sapiens
ASTKGPSVFFLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 106 aa, Homo sapiens
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 6 - protein, 105 aa, Homo sapiens
QPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 7 - protein, 4 aa, Artificial Sequence
  Description of Artificial Sequence Synthetic peptide
FGXG

SEQ ID NO: 8 - protein, 5 aa, Artificial Sequence
  Description of Artificial Sequence Synthetic peptide
LEWIG

SEQ ID NO: 9 - protein, 4 aa, Artificial Sequence
  Description of Artificial Sequence Synthetic peptide
WGXG

SEQ ID NO: 10 - protein, 9 aa, Artificial Sequence
  Description of Artificial Sequence Synthetic peptide
CXXXXXXXX

SEQ ID NO: 11 - protein, 6 aa, Artificial Sequence
  Description of Artificial Sequence Synthetic peptide
GGGGSG

SEQ ID NO: 12 - protein, 5 aa, Artificial Sequence
  Description of Artificial Sequence Synthetic peptide
GGSGG

SEQ ID NO: 13 - protein, 10 aa, Artificial Sequence
  Description of Artificial Sequence Synthetic peptide
GGGGSGGGGS

SEQ ID NO: 14 - protein, 9 aa, Artificial Sequence
  Description of Artificial Sequence Synthetic peptide
GGSGGGGSG

SEQ ID NO: 15 - protein, 10 aa, Artificial Sequence
  Description of Artificial Sequence Synthetic peptide
GGSGGGGSGS

SEQ ID NO: 16 - protein, 13 aa, Artificial Sequence
  Description of Artificial Sequence Synthetic peptide
GGSGGGGSGGGGS

SEQ ID NO: 17 - protein, 14 aa, Artificial Sequence
  Description of Artificial Sequence Synthetic peptide
GGGGSGGGGSGGGG

SEQ ID NO: 18 - protein, 15 aa, Artificial Sequence
  Description of Artificial Sequence Synthetic peptide
GGGGSGGGGSGGGGS

SEQ ID NO: 19 - protein, 6 aa, Artificial Sequence
  Description of Artificial Sequence Synthetic peptide
ASTKGP

SEQ ID NO: 20 - protein, 13 aa, Artificial Sequence
  Description of Artificial Sequence Synthetic peptide
ASTKGPSVFPLAP

SEQ ID NO: 21 - protein, 5 aa, Artificial Sequence
  Description of Artificial Sequence Synthetic peptide
TVAAP

SEQ ID NO: 22 - protein, 6 aa, Artificial Sequence
  Description of Artificial Sequence Synthetic peptide
RTVAAP

SEQ ID NO: 23 - protein, 12 aa, Artificial Sequence
  Description of Artificial Sequence Synthetic peptide
TVAAPSVFIFPP

SEQ ID NO: 24 - protein, 13 aa, Artificial Sequence
  Description of Artificial Sequence Synthetic peptide
RTVAAPSVFIFPP

SEQ ID NO: 25 - protein, 16 aa, Artificial Sequence
  Description of Artificial Sequence Synthetic peptide
AKTTPKLEEGEFSEAR

SEQ ID NO: 26 - protein, 17 aa, Artificial Sequence
  Description of Artificial Sequence Synthetic peptide
AKTTPKLEEGEFSEARV

SEQ ID NO: 27 - protein, 9 aa, Artificial Sequence
  Description of Artificial Sequence Synthetic peptide
AKTTPKLGG

SEQ ID NO: 28 - protein, 10 aa, Artificial Sequence
  Description of Artificial Sequence Synthetic peptide
SAKTTPKLGG

SEQ ID NO: 29 - protein, 6 aa, Artificial Sequence
  Description of Artificial Sequence Synthetic peptide
SAKTTP

SEQ ID NO: 30 - protein, 6 aa, Artificial Sequence
  Description of Artificial Sequence Synthetic peptide
RADAAP

SEQ ID NO: 31 - protein, 9 aa, Artificial Sequence
  Description of Artificial Sequence Synthetic peptide
RADAAPTVS

SEQ ID NO: 32 - protein, 12 aa, Artificial Sequence
  Description of Artificial Sequence Synthetic peptide
RADAAAAGGPGS

SEQ ID NO: 33 - protein, 27 aa, Artificial Sequence
  Description of Artificial Sequence Synthetic peptide
RADAAAAGGGGSGGGGSGGGGSGGGGS

SEQ ID NO: 34 - protein, 18 aa, Artificial Sequence
  Description of Artificial Sequence Synthetic peptide
SAKTTPKLEEGEFSEARV

SEQ ID NO: 35 - protein, 5 aa, Artificial Sequence
  Description of Artificial Sequence Synthetic peptide
ADAAP

SEQ ID NO: 36 - protein, 12 aa, Artificial Sequence
  Description of Artificial Sequence Synthetic peptide
ADAAPTVSIFPP

SEQ ID NO: 37 - protein, 6 aa, Artificial Sequence
  Description of Artificial Sequence Synthetic peptide
QPKAAP

SEQ ID NO: 38 - protein, 13 aa, Artificial Sequence
  Description of Artificial Sequence Synthetic peptide
QPKAAPSVTLFPP

SEQ ID NO: 39 - protein, 6 aa, Artificial Sequence
  Description of Artificial Sequence Synthetic peptide
AKTTPP

SEQ ID NO: 40 - protein, 13 aa, Artificial Sequence
  Description of Artificial Sequence Synthetic peptide
AKTTPPSVTPLAP

SEQ ID NO: 41 - protein, 6 aa, Artificial Sequence
  Description of Artificial Sequence Synthetic peptide
AKTTAP

SEQ ID NO: 42 - protein, 13 aa, Artificial Sequence
  Description of Artificial Sequence Synthetic peptide
AKTTAPSVYPLAP

SEQ ID NO: 43 - protein, 15 aa, Artificial Sequence
  Description of Artificial Sequence Synthetic peptide
GENKVEYAPALMALS

SEQ ID NO: 44 - protein, 15 aa, Artificial Sequence
  Description of Artificial Sequence Synthetic peptide
GPAKELTPLKEAKVS

SEQ ID NO: 45 - protein, 15 aa, Artificial Sequence
  Description of Artificial Sequence Synthetic peptide
GHEAAAVMQVQYPAS

SEQ ID NO: 46 - protein, 247 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSASTKGPQVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSS

SEQ ID NO: 47 - protein, 119 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSS

SEQ ID NO: 48 - protein, 6 aa, Artificial Sequence
  Description of Artificial Sequence Synthetic peptide
ASTKGP

SEQ ID NO: 49 - protein, 122 aa, Artificial Sequence
  Description of Artificial Sequence Synthetic polypeptide
QVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSS

SEQ ID NO: 50 - protein, 330 aa, Artificial Sequence; identical to SEQ ID NO: 4
  Description of Artificial Sequence Synthetic polypeptide

SEQ ID NO: 51 - protein, 221 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITRTVAAPDIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKR

SEQ ID NO: 52 - protein, 108 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITR

SEQ ID NO: 53 - protein, 5 aa, Artificial Sequence
  Description of Artificial Sequence Synthetic peptide
TVAAP

SEQ ID NO: 54 - protein, 108 aa, Artificial Sequence
  Description of Artificial Sequence Synthetic polypeptide
DIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKR

SEQ ID NO: 55 - protein, 106 aa, Artificial Sequence; identical to SEQ ID NO: 5
  Description of Artificial Sequence Synthetic polypeptide

SEQ ID NO: 56 - protein, 254 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSASTKGPSVFPLAPQVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSS

SEQ ID NO: 57 - protein, 119 aa, Artificial Sequence; identical to SEQ ID NO: 47
  Description of Artificial Sequence Synthetic polypeptide

SEQ ID NO: 58 - protein, 13 aa, Artificial Sequence
  Description of Artificial Sequence Synthetic peptide
ASTKGPSVFPLAP

SEQ ID NO: 59 - protein, 122 aa, Artificial Sequence; identical to SEQ ID NO: 49
  Description of Artificial Sequence Synthetic polypeptide

SEQ ID NO: 60 - protein, 330 aa, Artificial Sequence; identical to SEQ ID NO: 4
  Description of Artificial Sequence Synthetic polypeptide

SEQ ID NO: 61 - protein, 228 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITRTVAAPSVFIFPPDIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKR

SEQ ID NO: 62 - protein, 108 aa, Artificial Sequence; identical to SEQ ID NO: 52
  Description of Artificial Sequence Synthetic polypeptide

SEQ ID NO: 63 - protein, 12 aa, Artificial Sequence
  Description of Artificial Sequence Synthetic peptide
TVAAPSVFIFPP

SEQ ID NO: 64 - protein, 108 aa, Artificial Sequence; identical to SEQ ID NO: 54
  Description of Artificial Sequence Synthetic polypeptide

SEQ ID NO: 65 - protein, 106 aa, Artificial Sequence; identical to SEQ ID NO: 5
  Description of Artificial Sequence Synthetic polypeptide

SEQ ID NO: 66 - protein, 247 aa, Artificial Sequence
  Description of Artificial Sequence Synthetic polypeptide
QVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSSASTKGPEVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSS

SEQ ID NO: 67 - protein, 122 aa, Artificial Sequence; identical to SEQ ID NO: 49
  Description of Artificial Sequence Synthetic polypeptide

SEQ ID NO: 68 - protein, 6 aa, Artificial Sequence
  Description of Artificial Sequence Synthetic peptide
ASTKGP

SEQ ID NO: 69 - protein, 119 aa, Artificial Sequence; identical to SEQ ID NO: 47
  Description of Artificial Sequence Synthetic polypeptide

SEQ ID NO: 70 - protein, 330 aa, Artificial Sequence; identical to SEQ ID NO: 4
  Description of Artificial Sequence Synthetic polypeptide

SEQ ID NO: 71 - protein, 221 aa, Artificial Sequence
  Description of Artificial Sequence Synthetic polypeptide
DIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKRTVAAPDIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITR

SEQ ID NO: 72 - protein, 108 aa, Artificial Sequence; identical to SEQ ID NO: 54
  Description of Artificial Sequence Synthetic polypeptide

SEQ ID NO: 73 - protein, 5 aa, Artificial Sequence
  Description of Artificial Sequence Synthetic peptide
TVAAP

SEQ ID NO: 74 - protein, 108 aa, Artificial Sequence; identical to SEQ ID NO: 52
  Description of Artificial Sequence Synthetic polypeptide

SEQ ID NO: 75 - protein, 106 aa, Artificial Sequence; identical to SEQ ID NO: 5
  Description of Artificial Sequence Synthetic polypeptide

SEQ ID NO: 76 - protein, 254 aa, Artificial Sequence
  Description of Artificial Sequence Synthetic polypeptide
QVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSSASTKGPSVFPLAPEVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSS

SEQ ID NO: 77 - protein, 122 aa, Artificial Sequence; identical to SEQ ID NO: 49
  Description of Artificial Sequence Synthetic polypeptide

SEQ ID NO: 78 - protein, 13 aa, Artificial Sequence
  Description of Artificial Sequence Synthetic peptide
ASTKGPSVFPLAP

SEQ ID NO: 79 - protein, 119 aa, Artificial Sequence; identical to SEQ ID NO: 47
  Description of Artificial Sequence Synthetic polypeptide

SEQ ID NO: 80 - protein, 330 aa, Artificial Sequence; identical to SEQ ID NO: 4
  Description of Artificial Sequence Synthetic polypeptide

SEQ ID NO: 81 - protein, 228 aa, Artificial Sequence
  Description of Artificial Sequence Synthetic polypeptide
DIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKRTVAAPSVFIFPPDIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITR

SEQ ID NO: 82 - protein, 108 aa, Artificial Sequence; identical to SEQ ID NO: 54
  Description of Artificial Sequence Synthetic polypeptide

SEQ ID NO: 83 - protein, 12 aa, Artificial Sequence
  Description of Artificial Sequence Synthetic peptide
TVAAPSVFIFPP

SEQ ID NO: 84 - protein, 108 aa, Artificial Sequence; identical to SEQ ID NO: 52
  Description of Artificial Sequence Synthetic polypeptide

SEQ ID NO: 85 - protein, 106 aa, Artificial Sequence; identical to SEQ ID NO: 5
  Description of Artificial Sequence Synthetic polypeptide

SEQ ID NO: 86 - protein, 247 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSASTKGPQVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTVSS

SEQ ID NO: 87 - protein, 119 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSS

SEQ ID NO: 88 - protein, 6 aa, Artificial Sequence
  Description of Artificial Sequence Synthetic peptide
ASTKGP

SEQ ID NO: 89 - protein, 122 aa, Artificial Sequence
  Description of Artificial Sequence Synthetic polypeptide
QVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTVSS

SEQ ID NO: 90 - protein, 330 aa, Artificial Sequence; identical to SEQ ID NO: 4
  Description of Artificial Sequence Synthetic polypeptide

SEQ ID NO: 91 - protein, 221 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQITRTVAAPDIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEIKR

SEQ ID NO: 92 - protein, 108 aa, Artificial Sequence; identical to SEQ ID NO: 52
  Description of Artificial Sequence Synthetic polypeptide

SEQ ID NO: 93 - protein, 5 aa, Artificial Sequence
  Description of Artificial Sequence Synthetic peptide
TVAAP

SEQ ID NO: 94 - protein, 108 aa, Artificial Sequence
  Description of Artificial Sequence Synthetic polypeptide
DIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEIKR

SEQ ID NO: 95 - protein, 106 aa, Artificial Sequence; identical to SEQ ID NO: 5
  Description of Artificial Sequence Synthetic polypeptide

SEQ ID NO: 96 - protein, 247 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTTVTVSSASTKGPQVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSS

SEQ ID NO: 97 - protein, 119 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTTVTVSS

SEQ ID NO: 98 - protein, 6 aa, Artificial Sequence
  Description of Artificial Sequence Synthetic peptide
ASTKGP

SEQ ID NO: 99 - protein, 122 aa, Artificial Sequence; identical to SEQ ID NO: 49
  Description of Artificial Sequence Synthetic polypeptide

SEQ ID NO: 100 - protein, 330 aa, Artificial Sequence; identical to SEQ ID NO: 4
  Description of Artificial Sequence Synthetic polypeptide

SEQ ID NO: 101 - protein, 221 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKRTVAAPDIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKR

SEQ ID NO: 102 - protein, 108 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKR

SEQ ID NO: 103 - protein, 5 aa, Artificial Sequence
  Description of Artificial Sequence Synthetic peptide
TVAAP

SEQ ID NO: 104 - protein, 108 aa, Artificial Sequence; identical to SEQ ID NO: 54
  Description of Artificial Sequence Synthetic polypeptide

SEQ ID NO: 105 - protein, 106 aa, Artificial Sequence; identical to SEQ ID NO: 5
  Description of Artificial Sequence Synthetic polypeptide

SEQ ID NO: 106 - protein, 247 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRAEDTAVYYCARGGVYKGYFDVWGQGTPVTVSSASTKGPQVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSS

SEQ ID NO: 107 - protein, 119 aa, Artificial Sequence; identical to SEQ ID NO: 87
  Description of Artificial Sequence Synthetic polypeptide

SEQ ID NO: 108 - protein, 6 aa, Artificial Sequence
  Description of Artificial Sequence Synthetic peptide
ASTKGP

SEQ ID NO: 109 - protein, 122 aa, Artificial Sequence; identical to SEQ ID NO: 49
  Description of Artificial Sequence Synthetic polypeptide

SEQ ID NO: 110 - protein, 330 aa, Artificial Sequence; identical to SEQ ID NO: 4
  Description of Artificial Sequence Synthetic polypeptide

SEQ ID NO: 111 - protein, 221 aa, Artificial Sequence; identical to SEQ ID NO: 51
  Description of Artificial Sequence Synthetic polypeptide

SEQ ID NO: 112 - protein, 108 aa, Artificial Sequence; identical to SEQ ID NO: 52
  Description of Artificial Sequence Synthetic polypeptide

SEQ ID NO: 113 - protein, 5 aa, Artificial Sequence
  Description of Artificial Sequence Synthetic peptide
TVAAP

SEQ ID NO: 114 - protein, 108 aa, Artificial Sequence; identical to SEQ ID NO: 54
  Description of Artificial Sequence Synthetic polypeptide

SEQ ID NO: 115 - protein, 106 aa, Artificial Sequence; identical to SEQ ID NO: 5
  Description of Artificial Sequence Synthetic polypeptide

SEQ ID NO: 116 - protein, 247 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTPVTVSSASTKGPQVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTVSS

SEQ ID NO: 117 - protein, 119 aa, Artificial Sequence; identical to SEQ ID NO: 47
  Description of Artificial Sequence Synthetic polypeptide

SEQ ID NO: 118 - protein, 6 aa, Artificial Sequence
  Description of Artificial Sequence Synthetic peptide
ASTKGP

SEQ ID NO: 119 - protein, 122 aa, Artificial Sequence; identical to SEQ ID NO: 89
  Description of Artificial Sequence Synthetic polypeptide

SEQ ID NO: 120 - protein, 330 aa, Artificial Sequence; identical to SEQ ID NO: 4
  Description of Artificial Sequence Synthetic polypeptide

SEQ ID NO: 121 - protein, 221 aa, Artificial Sequence; identical to SEQ ID NO: 91
  Description of Artificial Sequence Synthetic polypeptide

SEQ ID NO: 122 - protein, 108 aa, Artificial Sequence; identical to SEQ ID NO: 52
  Description of Artificial Sequence Synthetic polypeptide

SEQ ID NO: 123 - protein, 5 aa, Artificial Sequence
  Description of Artificial Sequence Synthetic peptide
TVAAP

SEQ ID NO: 124 - protein, 108 aa, Artificial Sequence; identical to SEQ ID NO: 94
  Description of Artificial Sequence Synthetic polypeptide

SEQ ID NO: 125 - protein, 106 aa, Artificial Sequence; identical to SEQ ID NO: 5
  Description of Artificial Sequence Synthetic polypeptide

SEQ ID NO: 126 - protein, 254 aa, Artificial Sequence
  Description of Artificial Sequence Synthetic polypeptide
EVQLVESGGGVVQPGRSLRLSCSASGFIFSRYDMSWVRQAPGKGLEWVAYISHGGAGTYYPDSVKGRFTISRDNSKNTLFLQMDSLRPEDTGVYFCARGGVTKGYFDVWGQGTTVTVSSASTKGPSVFPLAPQVQLVESGGGVVQPGRSLRLSCTASGFTFSMFGVHWVRQAPGKGLEWVAAVSYDGSNKYYAESVKGRFTISRDNSKNILFLQMDSLRLEDTAVYYCARGRPKVVIPAPLAHWGQGTLVTFSS

SEQ ID NO: 127 - protein, 119 aa, Artificial Sequence; identical to SEQ ID NO: 97
  Description of Artificial Sequence Synthetic polypeptide

SEQ ID NO: 128 - protein, 13 aa, Artificial Sequence
  Description of Artificial Sequence Synthetic peptide
ASTKGPSVFPLAP

SEQ ID NO: 129 - protein, 122 aa, Artificial Sequence; identical to SEQ ID NO: 49
  Description of Artificial Sequence Synthetic polypeptide

SEQ ID NO: 130 - protein, 330 aa, Artificial Sequence; identical to SEQ ID NO: 4
  Description of Artificial Sequence Synthetic polypeptide

SEQ ID NO: 131 - protein, 228 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLEIKRTVAAPSVFIFPPDIQMTQSPSSVSASVGDRVTITCRASQGISSWLAWYQQKPGKAPKLLIYEASNLETGVPSRFSGSGSGSDFTLTISSLQPEDFATYYCQQTSSFLLSFGGGTKVEHKR

SEQ ID NO: 132 - protein, 108 aa, Artificial Sequence; identical to SEQ ID NO: 102
  Description of Artificial Sequence Synthetic polypeptide

SEQ ID NO: 133 - protein, 12 aa, Artificial Sequence
  Description of Artificial Sequence Synthetic peptide
TVAAPSVFIFPP

SEQ ID NO: 134 - protein, 108 aa, Artificial Sequence; identical to SEQ ID NO: 54
  Description of Artificial Sequence Synthetic polypeptide

SEQ ID NO: 135 - protein, 106 aa, Artificial Sequence; identical to SEQ ID NO: 5
  Description of Artificial Sequence Synthetic polypeptide

SEQ ID NO: 136 - protein, 107 aa, Artificial Sequence
  Description of Artificial Sequence Synthetic polypeptide
DIQMTQSPSSLSASVGDRVTITCRASGNIHNYLTWYQQTPGKAPKLLIYNAKTLADGVPSRFSGSGSGTDYTFTISSLQPEDIATYYCQHFWSIPYTFGQGTKLQIT

SEQ ID NO: 137 - protein, 107 aa, Artificial Sequence; identical to SEQ ID NO: 136
  Description of Artificial Sequence Synthetic polypeptide

(19) United States
(12) Patent Application Publication
(10) Pub. No.: US 2015/0203592 A1
(43) Pub. Date: Jul. 23, 2015

(54) COMPOSITIONS AND METHODS FOR TREATING OSTEOARTHRITIS

(71) Applicant: AbbVie Inc., North Chicago, IL (US)

(72) Inventor: Susanne X. Wang, Worcester, MA (US)

(21) Appl. No.: 14/558,602

(22) Filed: Dec. 2, 2014

Related U.S. Application Data
(60) Provisional application No. 62/049,820, filed on Sep. 12, 2014.
(60) Provisional application No. 62/008,987, filed on Jun. 6, 2014.
(60) Provisional application No. 61/981,589, filed on Apr. 18, 2014.
(60) Provisional application No. 61/970,243, filed on Mar. 25, 2014.
(60) Provisional application No. 61/939,673, filed on Feb. 13, 2014.
(60) Provisional application No. 61/934,432, filed on Jan. 31, 2014.
(60) Provisional application No. 61/910,804, filed on Dec. 2, 2013.

(51) Int. Cl. C07K 16/46; C07K 16/24

(52) U.S. Cl. CPC C07K 16/468; C07K 16/245; C07K 2317/31; C07K 2317/56; A61K 2039/505

(57) ABSTRACT

The present invention relates to the treatment of osteoarthritis in a human subject using anti-IL-1α and anti-IL-1β DVD-Ig proteins. In various embodiments, the osteoarthritis includes knee osteoarthritis or hand osteoarthritis.

### RELATED APPLICATIONS

The instant application claims the benefit and priority to U.S. Provisional Application Ser. No. 62/049,820 filed Sep. 12, 2014; U.S. Provisional Application Ser. No. 62/008,987 filed Jun. 6, 2014; U.S. Provisional Application Ser. No. 61/981,589 filed Apr. 18, 2014, U.S. Provisional Application Ser. No. 61/970,243 filed Mar. 25, 2014, U.S. Provisional Application Ser. No. 61/939,673 filed Feb. 13, 2014, U.S. Provisional Application Ser. No. 61/934,432 filed Jan. 31, 2014, and U.S. Provisional Application Ser. No. 61/910,804 filed Dec. 2, 2013, the contents of which for all of the foregoing are hereby incorporated by reference in their entireties.

### FIELD OF THE INVENTION

The present invention relates to the treatment of osteoarthritis in a human subject, and more specifically to the use of proteins that bind IL-1α and/or IL-1β to treat osteoarthritis.

### BACKGROUND OF THE INVENTION

The articular cartilage, or “hyaline cartilage,” of healthy vertebrates (including humans and other mammals) is a semi-transparent, opalescent connective tissue characterized by a columnar growth pattern of chondrocytes in an extracellular matrix (ECM) composed predominantly of proteoglycans, type II collagen, and water. Articular cartilage provides an effective weight-bearing cushion to prevent contact between opposing bones in a joint and thus is critical to the normal function of the joint. Articular cartilage is not only susceptible to damage by joint trauma, but also to a gradual process of erosion. Initially, such an erosion may be simply an asymptomatic “partial thickness defect” in which an area of reduced hyaline cartilage does not penetrate completely to the subchondral bone. Such partial thickness defects are usually not painful and typically are only detected during arthroscopic examination. However, if the erosive process is not treated, the base of a partial thickness defect may continue to wear away and the diameter of the defect may increase such that the defect eventually progresses to a “full thickness defect” that penetrates the underlying bone. Such full thickness defects may become sufficiently large that surfaces of opposing bones of the joint make contact and begin to erode one another, leading to inflammation, pain, and other degenerative changes, i.e., the classic symptoms of osteoarthritis. Osteoarthritis is thus a degenerative, progressive, and crippling disease that results in joint deformity, instability, impairment, and pain. Eventually, joint replacement surgery may be the only practical recourse for restoring, at least in part, some level of mobility to an individual.

A need remains for new and effective methods and compositions for treating individuals afflicted with osteoarthritis.

### SUMMARY OF THE INVENTION

The invention provides methods for treating osteoarthritis (OA) in a human subject. Such methods comprise administering to an individual (human or other mammal) one or more binding proteins that bind IL-1α and IL-1β. In an another embodiment, the invention provides methods for treating OA of a human subject using one or more of the binding proteins described herein that bind both IL-1α and IL-1β.

An aspect of the invention provides a method for reducing one or more symptoms of osteoarthritis (e.g., moderate-to-severe knee osteoarthritis and/or moderate-to-severe erosive hand osteoarthritis) in an individual comprising administering to the individual a binding protein that binds both IL-1α and IL-1β, wherein the binding protein is a dual variable domain immunoglobulin (DVD-Ig) binding protein including a variable heavy chain comprising an amino acid sequence selected from SEQ ID NOs: 46, 56, 66, 76, 86, 96, 106, 116, and 126, and including a variable light chain comprising an amino acid sequence selected from SEQ ID NOs: 51, 71, 81, 91, 101, 111, 121, and 131, whereby one or more symptoms of osteoarthritis is reduced.

In various embodiments, the individual suffers from a pain condition selected from the group consisting of allodynia, hyperalgesia, and a combination of allodynia and hyperalgesia.

In various embodiments, DVD-Ig binding protein binding to IL-1α and/or IL-1β prevents degradation or loss of cartilage.

In various embodiments, administering to said individual is subcutaneous administration or intravenous administration. In certain embodiments, a dosage of from about 0.1 mg/kg to about 10 mg/kg, from about 0.3 mg/kg to about 3 mg/kg, from about 1 mg/kg to about 3 mg/kg or at about 3 mg/kg can be administered. In certain embodiments, the binding protein is administered at a total dose of between about 1-25 mg, about 25-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, 300-325 mg, or about 325-350 mg of the binding protein. In certain embodiments, a total dose of between about 100 mg and about 200 mg is administered. In various embodiments, the dose is about 25 mg, 100 mg, or 200 mg.

In various embodiments, the binding protein is administered in a single dose. In other embodiments, the binding protein is administered in a multiple doses, e.g., every week, every other week, every three weeks or every four weeks. In certain embodiments, the binding protein is administered every other week. In certain embodiments, the binding protein is administered every four weeks.

In various embodiments, a decrease in one or more biomarker levels selected from the group consisting of high-sensitivity C-reactive protein (hsCRP), MMP degradation product type I (C1M), MMP degradation product type III (C3M) and C-reactive protein (CRPM) is observed in the subject after receiving one or more doses of the DVD-Ig binding protein relative to the one or more biomarker levels in the subject prior to receiving the one or more doses of the DVD-Ig binding protein.

In various embodiments, a decrease in one or more parameter levels selected from the group consisting of systemic inflammation, chronic tissue inflammation, inflammation-mediated tissue destruction, inflammation-mediated joint destruction, and connective tissue turnover is observed in the subject after receiving one or more doses of the DVD-Ig binding protein relative to the one or more parameter levels in the subject prior to receiving the one or more doses of the DVD-Ig binding protein.

In various embodiments, a decrease in one or more characteristics selected from the group consisting of pain, joint swelling, joint stiffness, effusion, rate of bone lesion, rate of joint space narrowing, rate of bony deformity formation, rate of bone sclerosis, synovitis, synovial hypertrophy, synovial hyperplasia, angiogenesis, and the presence of osteophytes is observed in the subject after receiving one or more doses of the DVD-Ig binding protein relative to the one or more characteristics in the subject prior to receiving the one or more doses of the DVD-Ig binding protein.

In various embodiments, an improvement in one or more metrics selected from the group consisting of Western Ontario and McMaster Universities Arthritis Index (WOMAC), Whole-Organ Magnetic Imaging Score (WORMS), Intermittent and Constant Osteoarthritis Pain (ICOAP) score; 11-point Numeric Rating Score (NRS) score, Physician Global Assessment of Disease Activity, Patient Reported Outcome, a Health Assessment Questionnaire (HAQ-DI), a patient global assessment of disease activity (VAS)), measurement or presence of an anti-drug antibody (ADA), tender joint count (TJC), swollen joint count (SJC), patient's assessment of pain, Work Instability Scale for Rheumatoid Arthritis, Short Form Health Survey (SF-36), American College of Rheumatology, ACR, (e.g., ACR20, ACR50, and ACR70); proportion of subjects achieving Low Disease Activity (LDA); Disease Activity Score 28 (DAS28; e.g., DAS28 based on C-reactive protein), Clinical Disease Activity Index (CDAI), simple disease activity index (SDAI), Clinical Remission criteria, and the individual's assessment (for example a questionnaire or a patient's global assessment) is observed in the subject after receiving one or more doses of the DVD-Ig binding protein relative to the one or more metrics in the subject prior to receiving the one or more doses.

An aspect of the invention provides a method for reducing pain associated with osteoarthritis (e.g., moderate-to-severe knee osteoarthritis and/or moderate-to-severe erosive hand osteoarthritis) in an individual, comprising administering to the individual a binding protein that binds both IL-1α and IL-1β, wherein the binding protein is a DVD-Ig binding protein including a variable heavy chain comprising an amino acid sequence selected from SEQ ID NOs: 46, 56, 66, 76, 86, 96, 106, 116, and 126, and including a variable light chain comprising an amino acid sequence selected from SEQ ID NOs: 51, 71, 81, 91, 101, 111, 121, and 131, whereby the pain is reduced.

In various embodiments, the individual suffers from a pain condition selected from the group consisting of allodynia, hyperalgesia, and a combination of allodynia and hyperalgesia.

In various embodiments, DVD-Ig binding protein binding to IL-1α and/or IL-1β prevents degradation or loss of cartilage.

In various embodiments, administering to said individual is subcutaneous administration or intravenous administration. In certain embodiments, a dosage of from about 0.1 mg/kg to about 10 mg/kg, from about 0.3 mg/kg to about 3 mg/kg, from about 1 mg/kg to about 3 mg/kg or at about 3 mg/kg can be administered. In certain embodiments, the binding protein is administered at a total dose of between about 1-25 mg, about 25-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, 300-325 mg, or about 325-350 mg of the binding protein. In certain embodiments, a total dose of between about 100 mg and about 200 mg is administered. In various embodiments, the dose is about 25 mg, 100 mg, or 200 mg.

In various embodiments, the binding protein is administered in a single dose. In other embodiments, the binding protein is administered in a multiple doses, e.g., every week, every other week, every three weeks or every four weeks. In certain embodiments, the binding protein is administered every other week. In certain embodiments, the binding protein is administered every four weeks.

In various embodiments, a decrease in one or more biomarker levels selected from the group consisting of high-sensitivity C-reactive protein (hsCRP), MMP degradation product type I (C1M), MMP degradation product type III (C3M) and C-reactive protein (CRPM) is observed in the subject after receiving one or more doses of the DVD-Ig binding protein relative to the one or more biomarker levels in the subject prior to receiving the one or more doses of the DVD-Ig binding protein.

In various embodiments, a decrease in one or more parameter levels selected from the group consisting of systemic inflammation, chronic tissue inflammation, inflammation-mediated tissue destruction, inflammation-mediated joint destruction, and connective tissue turnover is observed in the subject after receiving one or more doses of the DVD-Ig binding protein relative to the one or more parameter levels in the subject prior to receiving the one or more doses of the DVD-Ig binding protein.

In various embodiments, a decrease in one or more characteristics selected from the group consisting of pain, joint swelling, joint stiffness, effusion, rate of bone lesion, rate of joint space narrowing, rate of bony deformity formation, rate of bone sclerosis, synovitis, synovial hypertrophy, synovial hyperplasia, angiogenesis, and the presence of osteophytes is observed in the subject after receiving one or more doses of the DVD-Ig binding protein relative to the one or more characteristics in the subject prior to receiving the one or more doses of the DVD-Ig binding protein.

In various embodiments, an improvement in one or more metrics selected from the group consisting of WOMAC, WORMS, ICOAP score; 11-point NRS score, Physician Global Assessment of Disease Activity, Patient Reported Outcome, an HAQ-DI, a patient VAS, measurement or presence of an ADA, TJC, SJC, patient's assessment of pain, Work Instability Scale for Rheumatoid Arthritis, SF-36, ACR, (e.g., ACR20, ACR50, and ACR70); proportion of subjects achieving LDA; DAS28 (e.g., DAS28 based on C-reactive protein), CDAI, SDAI, clinical remission criteria, and the individual's assessment (for example a questionnaire or a patient's global assessment) is observed in the subject after receiving one or more doses of the DVD-Ig binding protein relative to the one or more metrics in the subject prior to receiving the one or more doses.

An aspect of the invention provides a method of reducing one or both of osteoarthritis (e.g., moderate-to-severe knee osteoarthritis and/or moderate-to-severe erosive hand osteoarthritis) and pain associated with osteoarthritis in a human subject, the method comprising administering to the human subject a binding protein that binds both IL-1α and IL-1β, wherein administering the binding protein is performed using a dose of between about 1 to about 3 mg/kg of weight of the binding protein to weight of the individual, or wherein administering the binding protein is performed using a dose of between about 100 mg and about 200 mg of the binding protein, and wherein a decrease in one or more biomarker levels selected from the group consisting of hsCRP, C1M, C3M and C-reactive protein CRPM is observed in the subject after receiving one or more doses of the binding protein that binds both IL-1α and IL-1β relative to the one or more biomarker levels in the subject prior to receiving the one or more doses of the binding protein that binds both IL-1α and IL-1β, to reduce one or both of the osteoarthritis and the pain associated with osteoarthritis.

In various embodiments, the binding protein comprises a DVD-Ig binding protein binding protein including a variable heavy chain comprising an amino acid sequence selected from SEQ ID NOs: 46, 56, 66, 76, 86, 96, 106, 116, and 126, and including a variable light chain comprising an amino acid sequence selected from SEQ ID NOs: 51, 71, 81, 91, 101, 111, 121, and 131.

In various embodiments, the individual suffers from a pain condition selected from the group consisting of allodynia, hyperalgesia, and a combination of allodynia and hyperalgesia.

In various embodiments, DVD-Ig binding protein binding to IL-1α and/or IL-1β prevents degradation or loss of cartilage.

In various embodiments, administering to said individual is subcutaneous administration or intravenous administration. In certain embodiments, a dosage of from about 0.1 mg/kg to about 10 mg/kg, from about 0.3 mg/kg to about 3 mg/kg, from about 1 mg/kg to about 3 mg/kg or at about 3 mg/kg can be administered. In certain embodiments, the binding protein is administered at a total dose of between about 1-25 mg, about 25-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, 300-325 mg, or about 325-350 mg of the binding protein. In certain embodiments, a total dose of between about 100 mg and about 200 mg is administered.

In various embodiments, the binding protein is administered in a single dose. In other embodiments, the binding protein is administered in a multiple doses, e.g., every week, every other week, every three weeks or every four weeks. In certain embodiments, the binding protein is administered every other week. In certain embodiments, the binding protein is administered every four weeks.

An aspect of the invention provides a method of decreasing one or more biomarker levels associated with osteoarthritis (e.g., moderate-to-severe knee osteoarthritis and/or moderate-to-severe erosive hand osteoarthritis) in a subject comprising administering to the subject a DVD-Ig binding protein binding protein including a variable heavy chain comprising an amino acid sequence selected from SEQ ID NOs: 46, 56, 66, 76, 86, 96, 106, 116, and 126, and including a variable light chain comprising an amino acid sequence selected from SEQ ID NOs: 51, 71, 81, 91, 101, 111, 121, and 131, wherein administering the binding protein is performed using a dose of between about 1 to about 3 mg/kg of weight of the binding protein to weight of the individual, or wherein administering the binding protein is performed using a dose of between about 100 mg and about 200 mg of the binding protein, and wherein a decrease in one or more biomarker levels selected from the group consisting of hsCRP, C1M, C3M and C-reactive protein CRPM is observed in the subject after administration of the DVD-Ig binding protein relative to the one or more biomarker levels in the subject prior to administration of the DVD-Ig binding protein, to decrease one or more biomarker levels associated with osteoarthritis.

In various embodiments, the individual suffers from a pain condition selected from the group consisting of allodynia, hyperalgesia, and a combination of allodynia and hyperalgesia.

In various embodiments, DVD-Ig binding protein binding to IL-1α and/or IL-1β prevents degradation or loss of cartilage.

In various embodiments, administering to said individual is subcutaneous administration or intravenous administration. In certain embodiments, a dosage of from about 0.1 mg/kg to about 10 mg/kg, from about 0.3 mg/kg to about 3 mg/kg, from about 1 mg/kg to about 3 mg/kg or at about 3 mg/kg can be administered. In certain embodiments, the binding protein is administered at a total dose of between about 1-25 mg, about 25-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, 300-325 mg, or about 325-350 mg of the binding protein. In certain embodiments, a total dose of between about 100 mg and about 200 mg is administered.

In various embodiments, the binding protein is administered in a single dose. In other embodiments, the binding protein is administered in a multiple doses, e.g., every week, every other week, every three weeks or every four weeks. In certain embodiments, the binding protein is administered every other week. In certain embodiments, the binding protein is administered every four weeks.

An aspect of the invention provides a method for treating osteoarthritis in a human subject comprising the step of administering to the human subject a binding protein that binds both IL-1α and IL-1β, wherein the binding protein is a DVD-Ig binding protein including a variable heavy chain comprising an amino acid sequence selected from SEQ ID NOs: 46, 56, 66, 76, 86, 96, 106, 116, and 126, and including a variable light chain comprising an amino acid sequence selected from SEQ ID NOs: 51, 71, 81, 91, 101, 111, 121, and 131, in a dose to achieve: (a) an area under the curve (AUC) of between about 5 and about 300 μg×day/mL; (b) a serum or plasma half-life (T½) of at least about 8 days; (c) a time point to maximum observed serum concentration (Tmax) of between about 2 days and about 8 days; and/or (d) a maximum observed serum concentration (Cmax) of between about 0.5 and about 25 μg/mL, following administration of the DVD-Ig binding protein to the human subject.

In various embodiments of the method, the AUC is between about 12 and about 280 μg×day/mL, the T½ is at least about 10 days, the Tmax is between about 2.5 days and about 7 days, and/or the Cmax is between about 0.1 and about 23 μg/mL. In other embodiments of the method, the AUC is at least about 30 μg×day/mL, the T½ is at least about 10 days, the Tmax is less than about 7 days, and/or the Cmax is at least about 2.5 μg/mL.

An aspect of the invention provides a method for treating pain associated with osteoarthritis in a human subject, wherein the pain is associated with osteoarthritis, the method comprising the step of administering to the human subject a binding protein that binds both IL-1α and IL-1β, wherein the binding protein is a DVD-Ig binding protein including a variable heavy chain comprising an amino acid sequence selected from SEQ ID NOs: 46, 56, 66, 76, 86, 96, 106, 116, and 126, and including a variable light chain comprising an amino acid sequence selected from SEQ ID NOs: 51, 71, 81, 91, 101, 111, 121, and 131, in a dose to achieve: (a) an AUC of between about 5 and about 300 μg×day/mL; (b) a T½ of at least about 8 days; (c) a Tmax of between about 2 days and about 8 days; and/or (d) a Cmax of between about 0.5 and about 25 μg/mL, following administration of the DVD-Ig binding protein to the human subject.

In various embodiments of the method, the AUC is between about 12 and about 280 μg×day/mL, the T½ is at least about 10 days, the Tmax is between about 2.5 days and about 7 days, and/or the Cmax is between about 0.1 and about 23 μg/mL. In other embodiments of the method, the AUC is at least about 30 μg×day/mL, the T½ is at least about 10 days, the Tmax is less than about 7 days, and/or the Cmax is at least about 2.5 μg/mL.

### BRIEF DESCRIPTION OF THE DRAWINGS

FIG. 1 panel A and FIG. 1 panel B are graphs showing the concentration of ABT-981 in serum (ordinate; μg/mL) of as a function of time (abscissa; days) for subjects intravenously (IV; FIG. 1 panel A) or subcutaneously (SC; FIG. 1 panel B) injected respectively. The healthy patients were administered ABT-981 binding protein at a dose of either: 0.3 mg/kg, 1 mg/kg, 3 mg/kg, or 10 mg/kg of ABT-981. FIG. 1 panel A and FIG. 1 panel B show the ABT-981 serum concentration-time profiles following a single dose, liner scale (mean+SD). [SD=standard deviation]

FIG. 2 panel A and FIG. 2 panel B are two graphs showing the mean dose-normalized Cₘₐₓ of ABT-981 in serum (ordinate; μg/mL/mg/kg) of as a function of dose (abscissa; mg/kg) for subjects intravenously (IV; left graph) or subcutaneously (SC; right graph) injected respectively in part 1 and part 2 of the pharmacokinetic studies described herein. The healthy patients were administered ABT-981 binding protein at a dose of either: 0.3 mg/kg, 1 mg/kg, 3 mg/kg, or 10 mg/kg of ABT-981. FIG. 2 panel A and FIG. 2 panel B show the mean+SD of ABT-981 dose normalized Cₘₐₓ and AUC following single doses after intravenous injection and subcutaneous injection respectively.

FIG. 3 is a line graph showing concentration of high-sensitivity C-reactive protein (hsCRP; mg/dl) as a function of time for knee OA patients administered ABT-981 DVD-Ig binding protein every two weeks (EOW E2W are used interchangeably). The knee OA patients were administered binding protein at a dose of either: 0.3 mg/kg (lower dose; Low Dose EOW), 1 mg/kg (middle dose; MID Dose EOW), or 3 mg/kg (higher dose; High Dose EOW). Control patients were administered a placebo.

FIG. 4 is a line graph showing change in baseline of MMP-generated fragment of type I collagen (C1M) as a function of time for knee OA patients administered EOW different amounts of ABT-981 DVD-Ig binding protein. The knee OA patients were administered binding protein at a dose of either: 0.3 mg/kg (lower dose; Low Dose EOW), 1 mg/kg (middle dose; MID Dose EOW), or 3 mg/kg (higher dose; High Dose EOW). Control patients were administered a placebo.

FIG. 5 is a line graph showing change in baseline of MMP-generated fragment of type III collagen (C3M) as a function of time for knee OA patients administered EOW different amounts of ABT-981 DVD-Ig binding protein. The knee OA patients were administered binding protein at a dose of either: 0.3 mg/kg (lower dose; Low Dose EOW), 1 mg/kg (middle dose; MID Dose EOW), or 3 mg/kg (higher dose; High Dose EOW). Control patients were administered a placebo.

FIG. 6 is a line graph showing change in baseline of C-reactive protein (CPRM) as a function of time for knee OA patients administered EOW different amounts of ABT-981 DVD-Ig binding protein. The knee OA were patients administered binding protein at a dose of either: 0.3 mg/kg (lower dose; Low Dose EOW), 1 mg/kg (middle dose; MID Dose EOW), or 3 mg/kg (higher dose; High Dose EOW). Control patients were administered a placebo.

FIG. 7 depicts the study design for a 52-week Phase II study for ABT-981 in treatment of subjects having symptomatic knee osteoarthritis. Subjects are screened and then analyzed for a period of time for changes in a number of indices/criteria prior to and after administration of doses of ABT-981 binding protein, for example physical function of the knee analyzed by Western Ontario and McMaster Universities (WOMAC) pain scale; intermittent and constant pain observed using the Intermittent and Constant Osteoarthritis Pain score; pain levels using a patient global assessment of arthritis; and knee synovitis/effusion volume, knee bone marrow lesions and extent of osteoarthritis as indicated by magnetic resonance imaging.

FIG. 8 is a graph showing relative mRNA change in IL-1α expression from baseline using delta CT analysis (ordinate) for samples from patients administered different doses (0.3, 1, and 3 mg/kg) of ABT-981 IL-1α/IL-1β DVD-Ig binding protein every other week. Control patients were administered a placebo every other week.

FIG. 9 is a graph showing relative mRNA change in IL-1β expression from baseline using delta CT analysis (ordinate) for samples from patients administered different doses (0.3, 1, and 3 mg/kg) of ABT-981 IL-1α/IL-1β DVD-Ig binding protein every other week. Control patients were administered a placebo every other week.

FIG. 10 is a graph showing ABT-981 serum concentration (μg/mL; ordinate) as a function of time (days; abscissa) for subjects administered different doses and regimens of ABT-981. The subjects were administered 0.3 mg/kg ABT-981 EOW, 1 mg/kg ABT-981 EOW, or 3 mg/kg ABT-981 EOW.

FIG. 11 is a graph showing ABT-981 serum concentration (μg/mL; ordinate) as a function of time (days; abscissa) for subjects administered 3 mg/kg ABT-981 E4W.

FIG. 12 is a drawing of the sensitive immuno PCR assay used to analyze IL-1α and IL-1β levels in serum of subjects administered ABT-981.

FIG. 13 is a schematic representation outlining an erosive hand osteoarthritis study.

### DETAILED DESCRIPTION OF THE INVENTION

The invention is based on the discovery that blocking the function of interleukin-1 (IL-1) can be an effective means to treat osteoarthritis (OA) in a human. According to the invention, blocking IL-1 function for treating OA may be achieved by administering to individual one or more binding proteins that bind IL-1α and IL-1β. Such a “dual-specific” therapy can be achieved by administering to an OA patient a binding protein (e.g., an antibody) that binds IL-1α and a binding protein (e.g., an antibody) that binds IL-1β or by administering a multivalent and multispecific binding protein that binds both IL-1α and IL-1β. Such a multivalent and multispecific binding protein useful in the invention includes a dual variable domain immunoglobulin binding protein (also referred herein as “DVD-Ig™” or “DVD-Ig” binding protein or molecule).

An aspect of the invention provides a method for treating osteoarthritis in an individual comprising the step of administering to the individual: a binding protein that binds both IL-1α and IL-1β, wherein the binding protein is a dual variable domain immunoglobulin (DVD-Ig) comprising first and second polypeptide chains, wherein the first polypeptide chain comprises a first VD1-(X1)n-VD2-C-(X2)n, wherein

VD1 is a first heavy chain variable domain;

VD2 is a second heavy chain variable domain;

C is a heavy chain constant domain;

X1 is a linker with the proviso that it is not CH1;

X2 is an Fc region;

n is 0 or 1; and

wherein the second polypeptide chain comprises a second VD1-(X1)n-VD2-C-(X2)n, wherein

VD1 is a first light chain variable domain;

VD2 is a second light chain variable domain;

C is a light chain constant domain;

X1 is a linker with the proviso that it is not CH1;

X2 does not comprise an Fc region;

n is 0 or 1.

wherein, the VD1-(X1)n-VD2 of the first polypeptide chain comprises an amino acid sequence selected from SEQ ID NOs: 46, 56, 66, 76, 86, 96, 106, 116, and 126, and the VD1-(X1)n-VD2 second polypeptide chain comprises a variable light chain comprising an amino acid sequence selected from SEQ ID NOs: 51, 71, 81, 91, 101, 111, 121, and 131. For example, the binding protein comprises a DVD-Ig binding protein as shown in Table 3. In various embodiments of the method, the binding protein further comprises at least one constant domain sequence. For example, the amino acid sequence for the constant region is described in Tables herein. In various embodiments of the method, the amino acid sequence comprises SEQ ID NOs: 3-6. In various embodiments of the method, the heavy chain constant region is selected from the group consisting of SEQ ID NOs: 50, 60, 70, 80, 90, 100, 110, 120, and 130. In various embodiments of the method, the light chain constant region is selected from the group consisting of SEQ ID NOs: 55, 65, 75, 85, 95, 105, 115, 125, and 135. In various embodiments, the individual is a human patient or human subject. In various embodiments, the binding protein neutralizes IL-1α and/or IL-1β. In various embodiments, the binding protein reduces activity of IL-1α and/or IL-1β.

In various embodiments, the binding protein binds both IL-1α and IL-1β and is formulated in a pharmaceutical composition comprising a pharmaceutically acceptable carrier. In various embodiments, the binding protein that binds both IL-1α and IL-1β is crystallized. In various embodiments, the crystallized binding protein is formulated in a composition comprising an ingredient and a polymeric carrier. For example, the polymeric carrier is a polymer selected from one or more of the group consisting of poly (acrylic acid), poly (cyanoacrylates), poly (amino acids), poly (anhydrides), poly (depsipeptide), poly (esters), poly (lactic acid), poly (lactic-co-glycolic acid) or PLGA, poly (b-hydroxybutryate), poly (caprolactone), poly (dioxanone); poly (ethylene glycol), poly (hydroxypropyl) methacrylamide, poly [(organo)phosphazene], poly (ortho esters), poly (vinyl alcohol), poly (vinylpyrrolidone), maleic anhydride-alkyl vinyl ether copolymers, pluronic polyols, albumin, alginate, cellulose and cellulose derivatives, collagen, fibrin, gelatin, hyaluronic acid, oligosaccharides, glycaminoglycans, sulfated polysaccharides, blends and copolymers thereof. In various embodiments, the ingredient is selected from the group consisting of albumin, sucrose, trehalose, lactitol, gelatin, hydroxypropyl-β-cyclodextrin, methoxypolyethylene glycol and polyethylene glycol.

The method in various embodiments further comprises administering to the individual a second agent that provides a desirable property. For example, the second agent is one or more compounds in the group consisting of budenoside, epidermal growth factor, corticosteroids, cyclosporin, sulfasalazine, aminosalicylates, 6-mercaptopurine, azathioprine, metronidazole, lipoxygenase inhibitors, mesalamine, olsalazine, balsalazide, antioxidants, thromboxane inhibitors, IL-1 receptor antagonists, anti-IL-1β monoclonal antibodies, anti-IL-6 monoclonal antibodies, growth factors, elastase inhibitors, pyridinyl-imidazole compounds, antibodies of TNF, LT, IL-2, IL-6, IL-7, IL-8, IL-12, IL-13, IL-15, IL-16, IL-18, IL-23, EMAP-II, GM-CSF, FGF, and PDGF, antibodies of CD2, CD3, CD4, CD8, CD-19, CD25, CD28, CD30, CD40, CD45, CD69, CD90 or their ligands, methotrexate, cyclosporin, FK506, rapamycin, mycophenolate mofetil, leflunomide, NSAIDs, ibuprofen, corticosteroids, prednisolone, phosphodiesterase inhibitors, adenosine agonists, antithrombotic agents, complement inhibitors, adrenergic agents, IRAK, NIK, IKK, p38, MAP kinase inhibitors, IL-1β converting enzyme inhibitors, TNFα converting enzyme inhibitors, T-cell signaling inhibitors, metalloproteinase inhibitors, sulfasalazine, azathioprine, 6-mercaptopurines, angiotensin converting enzyme inhibitors, soluble cytokine receptors, soluble p55 TNF receptor, soluble p75 TNF receptor, sIL-1RI, sIL-1RII, sIL-6R, anti-inflammatory cytokines, IL-4, IL-10, IL-11, IL-13, and TGF-β.

In various embodiments, the step of administering to said individual is by at least one mode of administration selected from: parenteral, subcutaneous, intramuscular, intravenous, intraarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, topical, oral, and transdermal. For example, the binding protein is subcutaneously administered as described in any of the working examples herein. Alternatively, the binding protein is intravenously administered as described in any of the working examples herein.

Administering the binding protein is performed in various embodiments at least two times or is performed periodically. For example the binding protein is administered at least two times, at least three times, or at least four times over a period of time. In various embodiments, the binding protein is administered multiple times to the individual over a period of days, weeks, months or years.

In various embodiments of the method, the binding protein is administered once per day, every other day, every week, every other week, every three weeks, every month, every two months, every few months or every six months.

Administering the binding protein is performed in various embodiments using a dose of at least: from 0.005 (milligrams per kilogram) mg/kg to 0.01 mg/kg, from 0.01 mg/kg to 0.05 mg/kg, from 0.05 mg/kg to 0.1 mg/kg, from 0.1 mg/kg to 0.5 mg/kg, from 0.5 mg/kg to 1 mg/kg, from 1 mg/kg to 2 mg/kg, from 2 mg/kg to 3 mg/kg, from 3 mg/kg to 4 mg/kg, from 4 mg/kg to 5 mg/kg, from 5 mg/kg to 6 mg/kg, from 6 mg/kg to 7 mg/kg, from 7 mg/kg to 8 mg/kg, from 8 mg/kg to 9 mg/kg, or from 9 mg/kg to 10 mg/kg of weight of the binding protein to weight of the individual. In various embodiments, the binding protein is administered at 0.3 mg/kg, 1 mg/kg, or 3 mg/kg.

In various embodiments of the method, the binding protein is administered using a single dose.

In various embodiments, the binding protein is administering using multiple doses. For example, the doses are administered multiple times using constant doses or ascending doses. Alternatively, the binding protein is administered multiple times using a descending dose.

In various embodiments, the method further comprises observing a reduction in an indicium of the osteoarthritis. In various embodiments, the method further comprises observing a reduction in a condition associated with the osteoarthritis. For example, the indicium or condition is presence of an osteophyte, bone sclerosis, effusion, joint swelling, synovitis, synovial hypertrophy and hyperplasia, angiogenesis, inflammation, stiffness, joint space narrowing, or pain associated with the osteoarthritis.

In various embodiments, the method further comprises observing or detecting a modulation (e.g., reduction or increase) in presence or activity of a biomarker. In various embodiments, the biomarker indicates presence or extent of the osteoarthritis. For example, the biomarker corresponds to presence of inflammation. In various embodiments of the method, the biomarker comprises at least one selected from the group consisting of: a carbohydrate; a peptide; a protein; and a genetic material. For example, the genetic material comprises DNA or RNA.

The biomarker comprises in various embodiments at least one selected from the group consisting of: a cell; a peptide or protein expressed by the cell; or a molecule that binds to the cell. In various embodiments of the method, the biomarker comprises a monocyte, a macrophage, B cells, T cells, a cytokine, (e.g., TNF, and IL-1Ra), a growth factor, an interleukin (e.g., IL-4, Il-6, IL-10, and IL-13), an osteoinductive factor, an interferon, a necrosis factor, a steroid, a proteoglycan, a fiber, a serum protein, an immunoglobulin, or a hormone. In various embodiments of the method, the biomarker comprises at least one selected from the group consisting of: a high-sensitivity C-reactive protein (hsCRP); a matrix metallopeptidase (MMP; for example MMP-9); a vascular endothelial growth factor (VEGF), a MMP degradation product for example MMP degradation product of type I, II, or III collagen (C1M, C2M, C3M); a C-reactive protein (CRPM), a prostaglandin, nitric oxide, a disintegrin and metalloproteinase with thrombospondin motifs (ADAMTS), an adipokine, an endothelial growth factor (EGF), a bone morphogenetic protein (BMP), a nerve growth factor (NGF), a substance P, an inducible Nitric Oxide Synthase (iNOS), CTX-I, CTX-II, TIINE, creatinine, and a vimentin (for example a citrullinated and MMP-degraded vimentin; VICM). In various embodiments, the biomarker comprises a local tissue degradation biomarker.

In various embodiments herein, observing or detecting the biomarker comprises obtaining a sample from the individual. In various embodiments, the sample is selected from: a cell, a fluid, and a tissue. For example, the fluid is at least one selected from: serum, plasma, synovial fluid, saliva, and urine. The cell or the tissue comprises for example at least one type selected from: vascular; epithelial; endothelial; dermal; connective; muscular; neuronal; soft tissue for example cartilage and collagen; bone; bone marrow; joint tissue; and an articular joint. For example, the biomarker is detected using an assay, a computer, or a probe. For example, the probe is a molecular probe that detects the presence of the biomarker. In an embodiment, the binding protein reduces the osteoarthritis and/or modulates (e.g., reduces and increases) expression and/or activity of the biomarker by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

In various embodiments, the binding protein produces localized effects. In various embodiments, the binding protein produces systemic effects.

In various embodiments of the method, the binding protein reduces the osteoarthritis in at least one metric or criteria from the group consisting of: Western Ontario and McMaster Universities Arthritis Index (WOMAC), Whole-Organ Magnetic Imaging Score (WORMS), Intermittent and Constant Osteoarthritis Pain (ICOAP) score; 11-point Numeric Rating Score (NRS) score, and the individual's assessment (for example a questionnaire or a patient's global assessment). In various embodiments, observing or evaluating is performed over a period of time selected from the group consisting of: hours, days, weeks, and months. In various embodiments, observing or evaluating determines that the binding protein does not produce adverse effects in the individual. In various embodiments of the method, observing or evaluating determines that the binding protein is at least one characteristic selected from the group consisting of: efficacious, therapeutic, safe, and producing beneficial biochemical and/or effects in the individual. In an embodiment, the binding protein reduces the osteoarthritis and/or modulates the metric by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

An aspect of the invention provides a method for treating pain associated with osteoarthritis, in which the method comprises the step of administering to the individual: a binding protein that binds both IL-1α and IL-1β, wherein the binding protein is a DVD-Ig binding protein comprises a variable heavy chain comprising SEQ ID NO: 46, and comprises a variable light chain comprising SEQ ID NO: 51. For example, the binding protein comprises a DVD-Ig binding protein shown in Table 3. In various embodiments of the method, the binding protein further comprises at least one constant domain sequence. For example, the amino acid sequence for the constant region is described in Tables herein. In various embodiments, the constant region amino acid sequence comprises SEQ ID NOs: 3-6. In various embodiments of the method, the heavy chain constant region is SEQ ID NO: 50, 100 or 130. In various embodiments of the method, the light chain constant region is SEQ ID NO: 55, 75, or 95.

In various embodiments of the method, the individual suffers from a pain condition selected from the group consisting of allodynia, hyperalgesia, and a combination of allodynia and hyperalgesia. For example the pain condition is associated with knee osteoarthritis or erosive hand osteoarthritis. In various embodiments of the method, the pain is nociceptive pain associated with osteoarthritis. For example, the pain is mechanical nociceptive pain associated with osteoarthritis.

In various embodiments of the method, the binding protein that binds both IL-1α and IL-1β is formulated in a pharmaceutical composition comprising a pharmaceutically acceptable carrier. In various embodiments of the method, the binding protein is crystallized. For example the crystallized binding protein that binds both IL-1α and IL-1β is formulated in a composition comprising an ingredient and a polymeric carrier. For example, the ingredient, when present, is for stabilizing the composition. In various embodiments of the method, the ingredient is selected from the group consisting of albumin, sucrose, trehalose, lactitol, gelatin, hydroxypropyl-β-cyclodextrin, methoxypolyethylene glycol and polyethylene glycol. In various embodiments of the method, the polymeric carrier is a polymer selected from one or more of the group consisting of poly (acrylic acid), poly (cyanoacrylates), poly (amino acids), poly (anhydrides), poly (depsipeptide), poly (esters), poly (lactic acid), poly (lactic-co-glycolic acid) or PLGA, poly (b-hydroxybutryate), poly (caprolactone), poly (dioxanone); poly (ethylene glycol), poly ((hydroxypropyl) methacrylamide, poly [(organo)phosphazene], poly (ortho esters), poly (vinyl alcohol), poly (vinylpyrrolidone), maleic anhydride-alkyl vinyl ether copolymers, pluronic polyols, albumin, alginate, cellulose and cellulose derivatives, collagen, fibrin, gelatin, hyaluronic acid, oligosaccharides, glycaminoglycans, sulfated polysaccharides, blends and copolymers thereof

In various embodiments, the method further comprises administering to the individual at least one additional agent, for example a second agent that provides a desirable property. In various embodiments of the method, the desired property is selected from one or more antibody parameters. In another embodiment, the antibody parameters are selected from the group consisting of antigen specificity, affinity to antigen, potency, biological function, epitope recognition, stability, solubility, production efficiency, immunogenicity, pharmacokinetics, bioavailability, tissue cross reactivity, and orthologous antigen binding. In various embodiments of the method, the second agent is one or more compounds in the group consisting of budenoside, epidermal growth factor, corticosteroids, cyclosporin, sulfasalazine, aminosalicylates, 6-mercaptopurine, azathioprine, metronidazole, lipoxygenase inhibitors, mesalamine, olsalazine, balsalazide, antioxidants, thromboxane inhibitors, IL-1 receptor antagonists, anti-IL-1β monoclonal antibodies, anti-IL-6 monoclonal antibodies, growth factors, elastase inhibitors, pyridinyl-imidazole compounds, antibodies of TNF, LT, IL-2, IL-6, IL-7, IL-8, IL-12, IL-13, IL-15, IL-16, IL-18, IL-23, EMAP-II, GM-CSF, FGF, and PDGF, antibodies of CD2, CD3, CD4, CD8, CD-19, CD25, CD28, CD30, CD40, CD45, CD69, CD90 or their ligands, methotrexate, cyclosporin, FK506, rapamycin, mycophenolate mofetil, leflunomide, NSAIDs, ibuprofen, corticosteroids, prednisolone, phosphodiesterase inhibitors, adenosine agonists, antithrombotic agents, complement inhibitors, adrenergic agents, IRAK, NIK, IKK, p38, MAP kinase inhibitors, IL-1β converting enzyme inhibitors, TNFα converting enzyme inhibitors, T-cell signaling inhibitors, metalloproteinase inhibitors, sulfasalazine, azathioprine, 6-mercaptopurines, angiotensin converting enzyme inhibitors, soluble cytokine receptors, soluble p55 TNF receptor, soluble p75 TNF receptor, sIL-1RI, sIL-1RII, sIL-6R, anti-inflammatory cytokines, IL-4, IL-10, IL-11, IL-13, and TIFF-β.

In various embodiments, the osteoarthritis comprises symptomatic osteoarthritis or radiographic osteoarthritis. In various embodiments of the method, the individual is suffering from knee osteoarthritis. In various embodiments of the method, the individual is suffering from hand osteoarthritis, e.g., erosive hand osteoarthritis.

In various embodiments of the method, administering the binding protein to said individual is by at least one mode selected from parenteral, subcutaneous, intramuscular, intravenous, intra-articular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, topical, oral, and transdermal.

In various embodiments of the method, the binding protein is administered using a single dose. In various embodiments of the method, administering the binding protein to said individual is performed periodically, for example at least two times over a period of hours, days, weeks or months. For example, administering is performed every two days, every four days, every week, or every two weeks. In various embodiments the binding protein is administered weekly, bi-weekly, monthly, bi-monthly, or semi-annually.

In various embodiments of the method, administering the binding protein is performed using a dose of at least: from 0.005 mg/kg to 0.01 mg/kg, from 0.01 mg/kg to 0.05 mg/kg, from 0.05 mg/kg to 0.1 mg/kg, from 0.1 mg/kg to 0.5 mg/kg, from 0.5 mg/kg to 1 mg/kg, from 1 mg/kg to 2 mg/kg, from 2 mg/kg to 3 mg/kg, from 3 mg/kg to 4 mg/kg, from 4 mg/kg to 5 mg/kg, from 5 mg/kg to 6 mg/kg, from 6 mg/kg to 7 mg/kg, from 7 mg/kg to 8 mg/kg, from 8 mg/kg to 9 mg/kg, or from 9 mg/kg to 10 mg/kg of weight of the binding protein to weight of the individual. In various embodiments of the method, the binding protein is administering using multiple doses, e.g., two or three doses over a period of time. For example, the dose is administered multiple times over a period of hours, days, weeks or months. In various embodiments of the method, the doses are administered every few hours, every day, every other day, every week, every other week, every month, every few months, or every year. In various embodiments of the method, the multiple doses administered are maintained constant. In various embodiments, multiple doses administered are modulated (i.e., increased or decreased dose relative to a prior dose). For example, the doses are modulated based on the binding protein treatment effects observed in the individual. For example, the doses are modulated based on presence or absence of a clinical indicator for effective treatment, and/or presence of absence of an indicator of an adverse effect in the individual.

The method in various embodiments further comprises observing or detecting a reduction in an indicium of the pain. For example the pain condition is associated with knee osteoarthritis or erosive hand osteoarthritis. In various embodiments of the method, the pain is nociceptive pain associated with osteoarthritis. For example, the pain is mechanical nociceptive pain associated with osteoarthritis.

The method in various embodiments further comprises measuring, observing or detecting presence or activity in a biomarker. In various the biomarker is a molecule that indicates presence or extent of the pain in the individual. For example the method involves measuring, observing or detecting a change in concentration or activity of the biomarker over a period of time. For example, the change is a reduction in amount of the biomarker. Alternatively, the change is an increase in amount of the biomarker. Alternatively, the measuring, observing or detecting comprises using an assay, a questionnaire, a strip, a well, a gel, a detector, an indicator, a dye, an imager, and a slide.

In various embodiments of the method, the biomarker comprises at least one selected from the group consisting of: a carbohydrate; a peptide; a protein; and a genetic material. For example, the genetic material comprises DNA or RNA. In various embodiments of the method, the biomarker comprises a growth factor, an interleukin, an osteoinductive factor, an interferon, a necrosis factor, a steroid, a proteoglycan, a fiber, a serum protein, an immunoglobulin, a hormone.

In various embodiments of the method, the biomarker comprises at least one selected from the group consisting of: a cell; a peptide or protein expressed by the cell; or a molecule that binds to the cell. For example, the biomarker is located in the serum or the cartilage of the individual. In various embodiments of the method, the biomarker comprises at least one selected from the group consisting of: a high-sensitivity C-reactive protein (hsCRP); a matrix metallopeptidase (MMP; for example MMP-9); a vascular endothelial growth factor (VEGF), a MMP degradation product for example a MMP degradation product of type I, II, or III collagen (C1M, C2M, C3M); a C-reactive protein (CRPM), CTX-I, CTX-II, TIINE, creatinine, and a vimentin (for example a citrullinated and MMP-degraded vimentin, VICM). In an embodiment, the binding protein reduces the osteoarthritis and/or modulates (e.g., reduces and increases) expression and/or activity of the biomarker by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

Measuring, observing or detecting the biomarker in various embodiments comprises obtaining a sample from the individual. For example, the sample is selected from: a cell, a fluid, and a tissue. In various embodiments of the method, the fluid is at least one selected from the group consisting of: serum, plasma, synovial fluid, saliva, and urine. In various embodiments of the method, the cell or the tissue is at least one selected from the group consisting of: vascular; epithelial; endothelial; dermal; connective; muscular; neuronal; soft tissue including cartilage and collagen; bone; bone marrow; joint tissue; and an articular joint. For example, the sample is collected after administering the binding protein and the biomarker is measured, observed or detecting. These biomarker data are then compared to biomarker data obtained from a control sample collected prior to the administering.

An aspect of the invention provides a method for treating osteoarthritis in a hand or a knee of an individual comprising the step of administering to the individual a DVD-Ig binding protein that binds both IL-1α and IL-1β, wherein a heavy chain comprises an amino acid sequence selected from SEQ ID NOs: 46, 56, 66, 76, 86, 96, 106, 116, and 126, and a variable light chain comprises an amino acid sequence selected from SEQ ID NOs: 51, 71, 81, 91, 101, 111, 121, and 131, wherein the binding protein is administered in an effective dose. In various embodiments, the binding protein further comprises at least one constant domain sequence. An amino acid sequence for the constant region is in various embodiments at least one of SEQ ID NOs: 3-6. In various embodiments of the method, the heavy chain constant region is selected from the group consisting of SEQ ID NO: 50, 60, 70, 80, 90, 100, 110, 120, and 130. In various embodiments of the method, the light chain constant region is SEQ ID NO: 55, 65, 75, 85, 95, 105, 115, 125, and 135.

An aspect of the invention provides a method for treating osteoarthritis in a hand or a knee of an individual and/or pain associated with the osteoarthritis, the method comprising: administering to the individual a DVD-Ig binding protein that binds both IL-1α and IL-1β, wherein the binding protein is a DVD-Ig comprises a variable heavy chain comprising SEQ ID NO: 46, and comprises a variable light chain comprising SEQ ID NO: 51, wherein administering the binding protein is performed for example using a dose of at least: from 0.005 mg/kg to 0.01 mg/kg, from 0.01 mg/kg to 0.05 mg/kg, from 0.05 mg/kg to 0.1 mg/kg, from 0.1 mg/kg to 0.5 mg/kg, from 0.5 mg/kg to 1 mg/kg, from 1 mg/kg to 2 mg/kg, from 2 mg/kg to 3 mg/kg, from 3 mg/kg to 4 mg/kg, from 4 mg/kg to 5 mg/kg, from 5 mg/kg to 6 mg/kg, from 6 mg/kg to 7 mg/kg, from 7 mg/kg to 8 mg/kg, from 8 mg/kg to 9 mg/kg, or from 9 mg/kg to 10 mg/kg of weight of the binding protein to weight of the individual.

Prior to administering the binding protein, the method comprises in various embodiments formulating or preparing a composition comprising the binding protein. For example, formulating or preparing comprises using a pharmaceutically acceptable carrier or buffer. In various embodiments, the composition is sterile. In various embodiments, the composition comprises a lyophilized material, or a re-constituted material from a lyophilized material. In various embodiments, the composition comprises a fluid for example a suspension. The binding protein in various embodiments comprises a crystallized protein or a conjugate.

In various embodiments of the method, administering the binding protein is by at least one mode selected from the group consisting of: parenteral, subcutaneous, intramuscular, intravenous, intra-articular, intraabdominal, intracapsular, intracartilaginous, intraosteal, intrapelvic, intraperitoneal, intrasynovial, intravesical, bolus, topical, oral, and transdermal.

In various embodiments of the method, administering the binding protein is performed periodically, for example at least two times. For example the binding protein is administered every week or every other week using a dose of at least: from 0.005 mg/kg to 0.01 mg/kg, from 0.01 mg/kg to 0.05 mg/kg, from 0.05 mg/kg to 0.1 mg/kg, from 0.1 mg/kg to 0.5 mg/kg, from 0.5 mg/kg to 1 mg/kg, from 1 mg/kg to 2 mg/kg, from 2 mg/kg to 3 mg/kg, from 3 mg/kg to 4 mg/kg, from 4 mg/kg to 5 mg/kg, from 5 mg/kg to 6 mg/kg, from 6 mg/kg to 7 mg/kg, from 7 mg/kg to 8 mg/kg, from 8 mg/kg to 9 mg/kg, or from 9 mg/kg to 10 mg/kg of weight of the binding protein to weight of the individual. In various embodiments the dose of the binding protein is administered weekly, bi-weekly, monthly, bi-monthly, or semi-annually.

The method further comprises in various embodiments observing reduction in an indicium of the osteoarthritis and/or the pain. The method further comprises in various embodiments observing a reduction in presence or activity of a biomarker. Generally, the biomarker indicates presence or extent of the osteoarthritis and/or the pain. For example, the biomarker comprises at least one selected from the group consisting of: a carbohydrate; a peptide; a protein; and a genetic material (e.g., DNA). In various embodiments, the biomarker comprises a growth factor, an interleukin, an osteoinductive factor, an interferon, a necrosis factor, a steroid, a proteoglycan, a fiber, a serum protein, an immunoglobulin, a hormone. In various embodiments, the biomarker comprises at least one selected from the group consisting of: a cell; a peptide or protein expressed by the cell; or a molecule that binds to the cell. In various embodiments of the method, the biomarker comprises at least one selected from the group consisting of: a high-sensitivity C-reactive protein; a matrix metallopeptidase; a vascular endothelial growth factor, a MMP degradation product for example a MMP degradation product of type I, II, or III collagen; a C-reactive protein, and a vimentin. In various embodiments of the method, the binding protein reduces the osteoarthritis and/or modulates (e.g., reduces and increases) expression and/or activity of the biomarker by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

In various embodiments of the method, observing comprises measuring or detecting the biomarker in a sample (e.g., a cell, a fluid, or a tissue) from the individual. For example fluid sample is at least one selected from: serum, plasma, synovial fluid, saliva, and urine. In various embodiments, the cell or the tissue is at least one selected from: vascular; epithelial; endothelial; dermal; connective; muscular; neuronal; soft tissue including cartilage and collagen; bone; bone marrow; joint tissue; and an articular joint.

The method further comprises in various embodiments observing that administering the binding protein produces one at least improved characteristic in the individual compared to another subject administered a control material or other substance.

In various embodiments, the individual prior to administering the binding protein is diagnosed to have inflammatory knee osteoarthritis. For example the inflammatory knee osteoarthritis comprises symptomatic, radiographic, and inflammatory knee osteoarthritis, and wherein administering the binding protein reduces the osteoarthritis and/or a negative condition associated with the osteoarthritis. For example the negative condition is at least one selected from the group consisting of: inflammation/swelling, pain, joint stiffness, effusion, and a bone lesion.

In various embodiments, diagnosing the osteoarthritis, and/or observing or evaluating the effectiveness of the binding protein comprises using a questionnaire, an interview, a procedure, a material, or an exam. For example, diagnosing or evaluating comprises using at least one metric or criteria selected from the group consisting of: Western Ontario and McMaster Universities Arthritis Index (WOMAC), Whole-Organ Magnetic Imaging Score (WORMS), Intermittent and Constant Osteoarthritis Pain (ICOAP) score; 11-point Numeric Rating Score (NRS) score, and the individual's assessment (for example a questionnaire or a patient's global assessment). In an embodiment, the binding protein reduces the osteoarthritis and/or modulates the metric or the criteria by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more. In various embodiments, observing or evaluating is performed over a period of time selected from the group consisting of: hours, days, weeks, and months. In various embodiments, observing or evaluating determines that the binding protein does not produce an adverse effect in the individual. In various embodiments of the method, observing or evaluating determines that the binding protein is at least one characteristic selected from the group consisting of: efficacious, therapeutic, safe, and producing beneficial biochemical and/or effects in the individual.

In various embodiments, administering the binding protein prevents further degradation or loss of cartilage in the joint. In an embodiment, the binding protein reduces the osteoarthritis and/or reduces cartilage degradation by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

In various embodiments, the method further comprises evaluating volume, thickness, composition, or appearance of the cartilage. For example, evaluating the cartilage involves using radiography, magnetic resonance imaging (MRI), ultrasound (US), and optical coherence tomography (OCT).

In various embodiments, administering the binding protein is performed using an effective dose for treating the osteoarthritis. For example, the effective dose is at least about: from 0.005 mg/kg to 0.01 mg/kg, from 0.01 mg/kg to 0.05 mg/kg, from 0.05 mg/kg to 0.1 mg/kg, from 0.1 mg/kg to 0.5 mg/kg, from 0.5 mg/kg to 1 mg/kg, from 1 mg/kg to 2 mg/kg, from 2 mg/kg to 3 mg/kg, from 3 mg/kg to 4 mg/kg, from 4 mg/kg to 5 mg/kg, from 5 mg/kg to 6 mg/kg, from 6 mg/kg to 7 mg/kg, from 7 mg/kg to 8 mg/kg, from 8 mg/kg to 9 mg/kg, from 9 mg/kg to 10 mg/kg, from 10 mg/kg to 12 mg/kg, or from 12 mg/kg to 15 mg/kg. In various embodiments of the method, administering the binding protein comprises at least: about 1-25 milligrams (mg), about 25-50 mg, about 50-75 mg, about 75-100 mg, about 100-125 mg, about 125-150 mg, about 150-175 mg, about 175-200 mg, about 200-225 mg, about 225-250 mg, about 250-275 mg, about 275-300 mg, 300-325 mg, or about 325-350 mg of the binding protein. In various embodiments of the method, administering the binding protein comprises contacting the subject with a 200 mg dose of the binding protein. In various embodiments, the dose is about 25 mg, 100 mg, or 200 mg.

In various embodiments, the administering the binding protein comprises intravenous administration. Alternatively, administering the binding protein comprises subcutaneous administration. In various embodiments prior to administering the binding protein, the method comprises formulating a composition comprising the binding protein. For example, formulating the composition comprises contacting the binding protein with a pharmaceutically acceptable carrier or buffer. In various embodiments, contacting is performed under sterile conditions.

In various embodiments of the method, administering the binding protein is performed subcutaneously or intravenously. In various embodiments, administering the binding protein comprises using 0.3, 1, 3, or 10 mg/kg of a binding protein described herein, e.g., DVD-Ig proteins found in Table 3.

In various embodiments of the method, administering the binding protein comprises contacting the subject with at least one dose of the binding protein over a period of time. In various embodiments of the method, the period of time is about a week, about every other week, every few weeks, about a month, or about every few months. For example, the period of time is about every other week.

In various embodiments of the method, the osteoarthritis comprises erosive hand osteoarthritis or another degenerative arthritis of the fingers and hand. In various embodiments of the method, the osteoarthritis is characterized by fusiform swelling of at least one joint of the hand. In various embodiments of the method, the osteoarthritis is characterized by Heberden's nodes and/or Bouchard's nodes. In various embodiments of the method, the osteoarthritis comprises symptomatic knee osteoarthritis. In various embodiments, the subject has or is suspected of having knee osteoarthritis or hand osteoarthritis.

In various embodiments, the method further comprises observing or detecting improvement of the subject's osteoarthritis. In various embodiments of the method, the improvement is determined or quantified by at least one metric or criteria selected from the group consisting of: American College of Rheumatology Criteria (ACR), Western Ontario and McMaster Universities Arthritis Index (WOMAC), Whole-Organ Magnetic Imaging Score (WORMS), Intermittent and Constant Osteoarthritis Pain (ICOAP) score; 11-point Numeric Rating Score (NRS) score, and the individual's assessment (for example a questionnaire or a patient's global assessment). In an embodiment, the binding protein reduces the osteoarthritis and/or modulates the metric by at least about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more. In various embodiments of the method, observing or detecting comprises analyzing serum of the subject.

In various embodiments, the dose achieves a serum metric or plasma metric. In various embodiments, the dose achieves a positive osteoarthritis metric or a pain metric. In various embodiments, the dose achieves a human therapeutic endpoint. For example, the therapeutic endpoint comprises reduction (e.g., 1-95%) in pain associated with osteoarthritis, cartilage degradation, inflammation/swelling, joint narrowing, joint stiffness, and effusion. In various embodiments, the therapeutic endpoint comprises an improvement in the metric/criteria described herein (e.g., ACR, WOMAC and ICOAP). For example, the improvement comprises about 1%, 3%, 5%, 7% 10%, 15%, 20%, 25%, 30%, 35%, 40%, 45%, 50%, 55%, 60%, 65%, 70%, 75%, 80%, 85%, 90%, 95%, 99% or more.

In various embodiments, the serum metric or the plasma metric is selected from the group of:

pharmacokinetics, absorption, bioavailability, distribution, metabolism, excretion, maximum observed concentration, and area under the curve. For example, the serum or the plasma metric (e.g., Cmax, Tmax, AUC, and half-life) is shown in a Table herein. In various embodiments, the Cmax and achieved is about 1-5 μg/mL, 5-10 μg/mL, 10-15 μg/mL, 15-20 μg/mL, and 20-25 μg/mL. In various embodiments, the AUC is about 20-275 μg·day/mL. For example, the AUC is about 20-50 μg·day/mL, 50-100 μg·day/mL, 100-150 μg·day/mL, 150-200 μg·day/mL, and 20-275 μg·day/mL at 0.3-3.0 mg/kg.

In various embodiments, the osteoarthritis metric or the pain metric is associated with one selected from the group consisting of: Physician Global Assessment of Disease Activity; Patient Reported Outcome; a Health Assessment Questionnaire (HAQ-DI); a patient global assessment of disease activity (VAS)); measurement or presence of an anti-drug antibody (ADA); tender joint count (TJC); swollen joint count (SJC); patient's assessment of pain; Work Instability Scale for Rheumatoid Arthritis; Short Form Health Survey (SF-36); American College of Rheumatology, ACR, (e.g., ACR20, ACR50, and ACR70); proportion of subjects achieving Low Disease Activity (LDA); Disease Activity Score 28 (DAS28; e.g., DAS28 based on C-reactive protein); Clinical Disease Activity Index (CDAI); simple disease activity index (SDAI); and Clinical Remission criteria.

An aspect of the invention provides a method of treating a human subject for osteoarthritis or pain associated with osteoarthritis, the method comprising administering a binding protein that binds both IL-1α and IL-1β, wherein administering the binding protein is performed using a dose of at least: from 0.005 (milligrams per kilogram) mg/kg to 0.01 mg/kg, from 0.01 mg/kg to 0.05 mg/kg, from 0.05 mg/kg to 0.1 mg/kg, from 0.1 mg/kg to 0.5 mg/kg, from 0.5 mg/kg to 1 mg/kg, from 1 mg/kg to 2 mg/kg, from 2 mg/kg to 3 mg/kg, from 3 mg/kg to 4 mg/kg, from 4 mg/kg to 5 mg/kg, from 5 mg/kg to 6 mg/kg, from 6 mg/kg to 7 mg/kg, from 7 mg/kg to 8 mg/kg, from 8 mg/kg to 9 mg/kg, or from 9 mg/kg to 10 mg/kg of weight of the binding protein to weight of the individual, wherein the dose achieves a serum metric, a plasma metric, an osteoarthritis metric, or a pain metric, whereby the osteoarthritis and/or the pain is treated. In various embodiments of the method, a heavy chain comprises an amino acid sequence selected from SEQ ID NOs: 46, 56, 66, 76, 86, 96, 106, 116, and 126, and a variable light chain comprises an amino acid sequence selected from SEQ ID NOs: 51, 71, 81, 91, 101, 111, 121, and 131. An aspect of the invention provides a method of treating a human subject for osteoarthritis or pain associated with osteoarthritis, the method comprising administering a binding protein that binds both IL-1α and IL-1β, wherein administering the binding protein is performed using a dose of at least: about 1-25 milligrams (mg), about 25-50 mg, about 50-75 mg, about 75-100 mg, about 100-125 mg, about 125-150 mg, about 150-175 mg, about 175-200 mg, about 200-225 mg, about 225-250 mg, about 250-275 mg, about 275-300 mg, 300-325 mg, or about 325-350 mg, wherein the dose achieves a serum metric, a plasma metric, an osteoarthritis metric, or a pain metric, whereby the osteoarthritis and/or the pain is treated.

In various embodiments of the method, a heavy chain comprises an amino acid sequence selected from SEQ ID NOs: 46, 56, 66, 76, 86, 96, 106, 116, and 126, and the amino acid sequence of a variable light chain comprising an amino acid sequence selected from SEQ ID NOs: 51, 71, 81, 91, 101, 111, 121, and 131. In various embodiments of the method, the binding protein is a DVD-Ig binding protein including a variable heavy chain comprising amino acid sequence SEQ ID NO: 46, and including a variable light chain comprising amino acid sequence SEQ ID NO: 51. In various embodiments, administering the binding protein increases tolerance to pain, for example mechanical nociceptive pain associated with osteoarthritis.

In various embodiments of the method, the serum or plasma metric is a characteristic selected from the group of: pharmacokinetics, absorption, bioavailability, distribution, metabolism, excretion, volume of distribution, clearance rate, peak concentration/maximum observed concentration (Cmax), and area under the curve (AUC). For example, the serum or plasma metric is described in the working examples described herein, e.g., AUC, Cmax, Tmax and AUCπ.

In various embodiments of the method, at least one pharmacokinetic characteristic selected from the group consisting of an AUC of between about 1 and about 30 μg·day/ml; a half-life of between about 1 and about 30 days; and/or a peak concentration (Cmax) of between about 1 and about 100 μg/ml, is achieved following administration of the anti-IL-Iα/β dual variable domain immunoglobulin, or antigen-binding portion thereof to the subject, thereby treating osteoarthritis in the subject. For example, the mean Cmax and AUCτ achieved is 2.59-22.6 μg/mL and 30.7-248 μg·day/mL at 0.3-3.0 mg/kg respectively. In various embodiments, the serum metric or plasma metric is found in Tables shown herein. For example, the Tmax is from one to three days, three days to seven days, seven to ten days, or ten days to 15 days after dosing. In various embodiments, the serum metric or plasma metric is mean terminal half-life. For example the mean terminal half-life is at least about one to three days, three days to five days, five days to seven days, seven days to ten days, ten days to 13 days, 13 days to 15 days, 15 days to 20 days, 20 days to 25 days, or 25 days to 30 days. In various embodiments of the method, the clearance rate of between about 0.01 to about 2 ml/h/kg. In various embodiments of the method, a volume of distribution of is effective for the binding protein to treat the osteoarthritis in the individual.

In various embodiments of the method, the osteoarthritis metric, the pain metric or the human therapeutic endpoint comprises one selected from the group consisting of: Physician Global Assessment of Disease Activity; Patient Reported Outcome; a Health Assessment Questionnaire (HAQ-DI); a patient global assessment of disease activity (VAS)); measurement or presence of an anti-drug antibody (ADA); tender joint count (TJC); swollen joint count (SJC); patient's assessment of pain; Work Instability Scale for Rheumatoid Arthritis; Short Form Health Survey (SF-36); American College of Rheumatology, ACR, (e.g., ACR20, ACR50, and ACR70); proportion of subjects achieving Low Disease Activity (LDA); Disease Activity Score 28 (DAS28; e.g., DAS28 based on C-reactive protein); Clinical Disease Activity Index (CDAI); simple disease activity index (SDAI); and Clinical Remission criteria.

In various embodiments, the step of administering to said individual is by at least one mode of administration selected from: parenteral, subcutaneous, intramuscular, intravenous, intraarticular, intrabronchial, intraabdominal, intracapsular, intracartilaginous, intracavitary, intracelial, intracerebellar, intracerebroventricular, intracolic, intracervical, intragastric, intrahepatic, intramyocardial, intraosteal, intrapelvic, intrapericardiac, intraperitoneal, intrapleural, intraprostatic, intrapulmonary, intrarectal, intrarenal, intraretinal, intraspinal, intrasynovial, intrathoracic, intrauterine, intravesical, bolus, vaginal, rectal, buccal, sublingual, intranasal, topical, oral, and transdermal. For example, the administration is subcutaneous administration. In an embodiment, administration is intravenous administration.

Administering the binding protein is performed in various embodiments at least two times or is performed periodically, for example at least two times, at least three times, or at least four times over a period of time. In various embodiments, the binding protein is administered multiple times to the individual over a period of days, weeks, months or years. For example, the binding protein is administered once per 12 hours, once per day, every other day, every week, every other week, every three weeks, every month, every two months, every few months or every six months.

The method further comprises in various embodiments observing that administering the binding protein produces one at least improved characteristic in the individual compared to another subject administered a control material or other substance.

In various embodiments, the individual prior to administering the binding protein is diagnosed to have inflammatory knee osteoarthritis. For example the inflammatory knee osteoarthritis comprises symptomatic, radiographic, and inflammatory knee osteoarthritis, and wherein administering the binding protein reduces the osteoarthritis and/or a negative condition associated with the osteoarthritis. For example the negative condition is at least one selected from the group consisting of: inflammation/swelling, pain, joint stiffness, effusion, and a bone lesion. In various embodiments, the individual prior to administering the binding protein is diagnosed to have hand osteoarthritis, for example erosive hand osteoarthritis.

In various embodiments of the method, the binding protein further comprises at least one constant domain sequence. For example, the amino acid sequence for the constant region is described in Tables herein (e.g., Table 2 and Table 3). In various embodiments, the amino acid sequence comprises SEQ ID NOs: 3-6, 50 or 55. In various embodiments of the method, the heavy chain constant region is selected from the group of SEQ ID NOs: 50, 60, 70, 80, 90, 100, 110, 120, and 130. In various embodiments of the method, the light chain constant region is selected from the group of SEQ ID NOs: 55, 65, 75, 85, 95, 105, 115, 125, and 135.

In various embodiments, the metric is associated with a biomarker that comprises at least one selected from the group consisting of: a cell; a peptide or protein expressed by the cell; or a molecule that binds to the cell.

In various embodiments of the method, the biomarker comprises at least one selected from the group consisting of: a high-sensitivity C-reactive protein; a matrix metallopeptidase; a vascular endothelial growth factor, a MMP degradation product for example a MMP degradation product of type I, II, or III collagen; a C-reactive protein, and a vimentin. In various embodiments of the method, the biomarker comprises a biomarker for tissue health, e.g., a biomarker for cartilage. For example the biomarker comprises a cartilage degradation biomarker.

Prior to administering the binding protein, the method comprises in various embodiments formulating or preparing a composition comprising the binding protein. For example, formulating or preparing comprises using a pharmaceutically acceptable carrier or buffer. In various embodiments, the composition is sterile. In various embodiments, the composition comprises a lyophilized material, or a re-constituted material from a lyophilized material. In various embodiments, the composition comprises a fluid for example a suspension. The binding protein in various embodiments comprises a crystallized protein or a conjugate.

### DEFINITIONS

Unless otherwise defined herein, scientific and technical terms used in connection with the present invention shall have the meanings that are commonly understood by those of ordinary skill in the art. The meaning and scope of the terms should be clear, however, in the event of any latent ambiguity, definitions provided herein take precedent over any dictionary or extrinsic definition. Further, unless otherwise required by context, singular terms shall include pluralities and plural terms shall include the singular. In this application the use of the term “or” means “and/or” unless stated otherwise. Furthermore, the use of the term “including”, as well as other forms, such as “includes” and “included”, is not limiting. Also, terms such as “element” or “component” encompass both elements and components comprising one unit and elements and components that comprise more than one subunit unless specifically stated otherwise.

Generally, nomenclatures used in connection with, and techniques of, cell and tissue culture, molecular biology, immunology, microbiology, genetics, protein and nucleic acid chemistry, and nucleic acid hybridization described herein are those well-known and commonly used in the art. The methods and techniques of the present invention are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclatures used in connection with, and the laboratory procedures and techniques of, analytical chemistry, synthetic organic chemistry, and medicinal and pharmaceutical chemistry described herein are those well-known and commonly used in the art. Standard techniques are used for chemical syntheses, chemical analyses, pharmaceutical preparation, formulation, and delivery, and treatment of patients.

That the present invention may be more readily understood, select terms are defined below.

The term “polypeptide” means any polymeric chain of amino acids. The terms “peptide” and “protein” are used interchangeably with the term polypeptide and also refer to a polymeric chain of amino acids. The term “polypeptide” encompasses native or artificial proteins, protein fragments and polypeptide analogs of a protein sequence. A polypeptide may be monomeric or polymeric.

The term “isolated protein” or “isolated polypeptide” means a protein or polypeptide that by virtue of its origin or source of derivation is not associated with naturally associated components that accompany it in its native state, is substantially free of other proteins from the same species, is expressed by a cell from a different species, or does not occur in nature. Thus, a polypeptide that is chemically synthesized or synthesized in a cellular system different from the cell from which it naturally originates will be “isolated” from its naturally associated components. A protein may also be rendered substantially free of naturally associated components by isolation, using protein purification techniques well known in the art.

The term “recovering” means the process of rendering a chemical species such as a polypeptide substantially free of naturally associated components by isolation, e.g., using protein purification techniques well known in the art.

The term “human IL-1α” (also abbreviated herein as “hIL-1α” or “IL-1α”), includes a pleiotropic cytokine involved in various immune responses, inflammatory processes, and hematopoiesis. For example, IL-1α includes the human cytokine produced by activated macrophages; it stimulates thymocyte proliferation by inducing IL-2 release, B-cell maturation and proliferation, and fibroblast growth factor activity. The term “human IL-1α” is intended to include recombinant human IL-1α (“rh IL-1α”) that can be prepared by standard recombinant expression methods.

The term “human IL-1 β” (also abbreviated herein as “hIL-1β” or “IL-1β”) includes a pleiotropic cytokine involved in various immune responses, inflammatory processes, and hematopoiesis. The term human “IL-1β” includes recombinant human IL-1 β (“rh IL-1β”) that can be prepared by standard recombinant expression methods.

The amino acid sequences of human IL-1α and IL-1β are shown in Table 1. See also U.S. Pat. No. 8,841,417 which issued Sep. 23, 2014 (U.S. publication number 2011/0280800 published Nov. 17, 2011) and U.S. Pat. No. 8,664,367 which issued Mar. 4, 2014 (U.S. publication number 2013/0195754 published Aug. 1, 2013), which are incorporated by reference herein in their entireties.

The term “biological activity” refers to all inherent biological properties of the cytokine. Biological properties of IL-1α and IL-1β include, but are not limited to, binding to an IL-1 receptor.

Biological properties of IL-1 include, but are not limited to, binding to the IL-1 receptor; stimulating thymocyte proliferation by inducing IL-2 release, B-cell maturation and proliferation, and fibroblast growth factor activity.

The terms “specific binding” or “specifically binding”, in reference to the interaction of an antibody, a protein, or a peptide with a second chemical species, mean that the interaction is dependent upon the presence of a particular structure (e.g., an antigenic determinant or epitope) on the chemical species, for example, an antibody recognizes and binds to a specific protein structure rather than to proteins generally. If an antibody is specific for epitope “A”, the presence of a molecule containing epitope A (or free, unlabeled A), in a reaction containing labeled “A” and the antibody, will reduce the amount of labeled A bound to the antibody.

TABLE 1
Sequences of Human IL-1α and IL-1β

| | Sequence | Sequence |
|---|---|---|
| Protein | Identifier | 123456789012345678901234567890 |
| Human pro IL-lα | SEQ ID NO: 1 | MAKVPDMFEDLKNCYSENEEDSSSIDHLSL |
| | | NQKSFYHVSYGPLHEGCMDQSVSLSISETS |
| | | KTSKLTFKESMVVVATNGKVLKKRRLSLSQ |
| | | SITDDDLEAIANDSEEEIIKPRSAPFSFLS |
| | | NVKYNFMRIIKYEFILNDALNQSIIRANDQ |
| | | YLTAAALHNLDEAVKFDMGAYKSSKDDAKI |
| | | TVILRISKTQLYVTAQDEDQPVLLKEMPEI |
| | | PKTITGSETNLLFFWETHGTKNYFTSVAHP |
| | | NLFIATKQDYWVCLAGGPPSITDFQILENQ |
| | | A |
| Human mature IL- | Residues 113-271 of | SAPFSFLSNVKYNFMRIIKYEFILNDALNQ |
| 1α | SEQ ID NO: 1 | SIIRANDQYLTAAALHNLDEAVKFDMGAYK |
| | | SSKDDAKITVILRISKTQLYVTAQDEDQPV |
| | | LLKEMPEIPKTITGSETNLLFFWETHGTKN |
| | | YFTSVAHPNLFIATKQDYWVCLAGGPPSIT |
| | | DFQILENQA |
| Human mature IL- | SEQ ID NO: 2 | APVRSLNCTLRDSQQKSLVMSGPYELKALH |
| 1β | | LQGQDMEQQVVFSMSFVQGEESNDKIPVAL |
| | | GLKEKNLYLSCVLKDDKPTLQLESVDPKNY |
| | | PKKKMEKRFVFNKIEINNKLEFESAQFPNW |
| | | YISTSQAENMPVFLGGTKGGQDITDFTMQF |
| | | VSS |

The term “antibody”, broadly refers to any immunoglobulin (Ig) molecule comprised of four polypeptide chains, two heavy (H) chains and two light (L) chains, or any functional fragment, mutant, variant, or derivative thereof, that retains the essential epitope binding features of an Ig molecule. Such mutant, variant, or derivative antibody formats are known in the art, non-limiting embodiments of which are discussed below.

In a full-length antibody, each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, and FR4 Immunoglobulin molecules can be of any type (e.g., IgG, IgE, IgM, IgD, IgA and IgY), class (e.g., IgG1, IgG2, IgG3, IgG4, IgA1 and IgA2) or subclass.

The term “antigen-binding portion” of an antibody refers to one or more fragments of an antibody that retain the ability to specifically bind to an antigen (e.g., hIL-1α). The antigen-binding function of an antibody can be performed by fragments of a full-length antibody. Such antibody embodiments may also have bispecific, dual specific or multi-specific formats, specifically binding to two or more different antigens. Examples of binding fragments encompassed within the term “antigen-binding portion” of an antibody include (i) an Fab fragment, which is a monovalent fragment consisting of the VL, VH, CL, and CH1 domains; (ii) an F(ab′)₂ fragment, which is a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody; (v) a dAb fragment (Ward et al. (1989) Nature 341:544-546, PCT Publication No. WO 90/05144), which comprises a single variable domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies (scFvs) are also intended to be encompassed within the term “antigen-binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, e.g., Holliger et al. (1993) Proc. Natl. Acad. Sci. USA 90:6444-6448; Poljak et al. (1994) Structure 2:1121-1123). Such antibody binding portions are known in the art (Kontermann and Dübel eds., Antibody Engineering (Springer-Verlag, New York, 2001) (ISBN 3-540-41354-5)).

The term “antibody construct” refers to a polypeptide comprising one or more the antigen binding portions of the invention linked to a linker polypeptide or an immunoglobulin constant domain. Linker polypeptides comprise two or more amino acid residues joined by peptide bonds and are used to link one or more antigen binding portions. Such linker polypeptides are well known in the art (see, e.g., Holliger et al. (1993) Proc. Natl. Acad. Sci. USA 90:6444-6448; Poljak et al. (1994) Structure 2:1121-1123). An immunoglobulin constant domain refers to a heavy or light chain constant domain. Human IgG heavy chain (gamma) and light chain (kappa and lambda) constant domain amino acid sequences are known in the art and represented in Table 2.

TABLE 2
Sequences of Human IgG Heavy and Light Chain Constant Domains

| | Sequence | Sequence |
|---|---|---|
| Protein | Identifier | 123456789012345678901234567890 |
| Ig gamma-1 | SEQ ID NO: 3 | ASTKGPSVFFLAPSSKSTSGGTAALGCLVK |
| constant region | | DYFPEPVTVSWNSGALTSGVHTFPAVLQSS |
| | | GLYSLSSVVTVPSSSLGTQTYICNVNHKPS |
| | | NTKVDKKVEPKSCDKTHTCPPCPAPELLGG |
| | | PSVFLFPPKPKDTLMISRTPEVTCVVVDVS |
| | | HEDPEVKFNWYVDGVEVHNAKTKPREEQYN |
| | | STYRVVSVLTVLHQDWLNGKEYKCKVSNKA |
| | | LPAPIEKTISKAKGQPREPQVYTLPPSREE |
| | | MTKNQVSLTCLVKGFYPSDIAVEWESNGQP |
| | | ENNYKTTPPVLDSDGSFFLYSKLTVDKSRW |
| | | QQGNVFSCSVMHEALHNHYTQKSLSLSPGK |
| Ig gamma-1 | SEQ ID NO: 4 | ASTKGPSVFPLAPSSKSTSGGTAALGCLVK |
| constant region | | DYFPEPVTVSWNSGALTSGVHTFPAVLQSS |
| mutant | | GLYSLSSVVTVPSSSLGTQTYICNVNHKPS |
| | | NTKVDKKVEPKSCDKTHTCPPCPAPEAAGG |
| | | PSVFLFPPKPKDTLMISRTPEVTCVVVDVS |
| | | HEDPEVKFNWYVDGVEVHNAKTKPREEQYN |
| | | STYRVVSVLTVLHQDWLNGKEYKCKVSNKA |
| | | LPAPIEKTISKAKGQPREPQVYTLPPSREE |
| | | MTKNQVSLTCLVKGFYPSDIAVEWESNGQP |
| | | ENNYKTTPPVLDSDGSFFLYSKLTVDKSRW |
| | | QQGNVFSCSVMHEALHNHYTQKSLSLSPGK |
| Ig Kappa | SEQ ID NO: 5 | TVAAPSVFIFPPSDEQLKSGTASVVCLLNN |
| constant region | | FYPREAKVQWKVDNALQSGNSQESVTEQDS |
| | | KDSTYSLSSTLTLSKADYEKHKVYACEVTH |
| | | QGLSSPVTKSFNRGEC |
| Ig Lambda | SEQ ID NO: 6 | QPKAAPSVTLFPPSSEELQANKATLVCLIS |
| constant region | | DFYPGAVTVAWKADSSPVKAGVETTTPSKQ |
| | | SNNKYAASSYLSLTPEQWKSHRSYSCQVTH |
| | | EGSTVEKTVAPTECS |

Still further, an antibody or antigen-binding portion thereof may be part of a larger immunoadhesion molecule, formed by covalent or noncovalent association of the antibody, or antigen binding portion thereof, with one or more other proteins or peptides. Examples of such immunoadhesion molecules include use of the streptavidin core region to make a tetrameric scFv molecule (Kipriyanov, S. et al. (1995) Human Antibod. Hybridomas 6:93-101) and use of a cysteine residue, a marker peptide and a C-terminal polyhistidine tag to make bivalent and biotinylated scFv molecules (Kipriyanov, S. et al. (1994) Mol. Immunol 31:1047-1058). Antigen binding portions of antibodies, such as Fab and F(ab′)₂fragments, can be prepared from whole antibodies using conventional techniques, such as papain or pepsin digestion, respectively, of whole antibodies. Moreover, antibodies, antigen binding portions thereof, and immunoadhesion molecules can be obtained using standard recombinant DNA techniques, as described herein.

An “isolated antibody” refers to an antibody that is substantially free of other antibodies having different antigenic specificities (e.g., an isolated antibody that specifically binds hIL-1α is substantially free of antibodies that specifically bind antigens other than hIL-1 α). An isolated antibody that specifically binds hIL-1 α may, however, have cross-reactivity to other antigens, such as IL-1 α molecules from other species. Moreover, an isolated antibody may be substantially free of other cellular material and/or chemicals.

The term “human antibody” includes antibodies having variable and constant regions derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody”, does not include antibodies in which CDR sequences derived from the germline of another mammalian species, such as a mouse, have been grafted onto human framework sequences.

The term “recombinant human antibody” includes all human antibodies that are prepared, expressed, created or isolated by recombinant means, such as antibodies expressed using a recombinant expression vector transfected into a host cell (described further in Section II C, below), antibodies isolated from a recombinant, combinatorial human antibody library (Hoogenboom, H. (1997) Trends Biotechnol. 15: 62-70; Azzazy and Highsmith (2002) Clin. Biochem. 35: 425-445; Gavilondo and Larrick (2000) BioTechniques 29:128-145; Hoogenboom and Chames (2000) Immunol Today 21: 371-378), antibodies isolated from an animal (e.g., a mouse) that is transgenic for human immunoglobulin genes (see, e.g., Taylor et al. (1992) Nucl. Acids Res. 20: 6287-6295; Kellermann and Green (2002) Curr. Opin. Biotechnol. 13: 593-597; Little et al. (2000) Immunol Today 21:364-370) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo.

The term “chimeric antibody” refers to antibodies that comprise heavy and light chain variable region sequences from one species and constant region sequences from another species, such as antibodies having murine heavy and light chain variable regions linked to human constant regions.

The term “CDR-grafted antibody” refers to antibodies that comprise heavy and light chain variable region sequences from one species but in which the sequences of one or more of the CDR regions of VH and/or VL regions are replaced with CDR sequences of another species, such as antibodies that have human heavy and light chain variable regions in which one or more of the human CDRs (e.g., CDR3) has been replaced with murine CDR sequences, for example, as obtained from a murine monoclonal antibody to human IL-1α.

As used herein, the term “CDR” refers to the complementarity determining region within antibody variable sequences. There are three CDRs in each of the variable regions of the heavy chain and the light chain, which are designated CDR1, CDR2, and CDR3, for each of the variable regions. The term “CDR set” as used herein refers to a group of three CDRs that occur in a single variable region (i.e., VH or VL) of an antigen binding site. The exact boundaries of these CDRs have been defined differently according to different systems. The system described by Kabat (Kabat et al. (1987, 1991) Sequences of Proteins of Immunological Interest (National Institutes of Health, Bethesda, Md.) not only provides an unambiguous residue numbering system applicable to any variable region of an antibody, but also provides precise residue boundaries defining the three CDRs. These CDRs may be referred to as Kabat CDRs. Chothia and coworkers (Chothia and Lesk (1987) J. Mol. Biol. 196: 901-917 and Chothia et al. (1989) Nature 342: 877-883) found that certain sub-portions within Kabat CDRs adopt nearly identical peptide backbone conformations, despite having great diversity at the level of amino acid sequence. These sub-portions were designated as L1, L2, and L3 or H1, H2, and H3, where the “L” and the “H” designates the light chain and the heavy chains regions, respectively. These regions may be referred to as Chothia CDRs, which have boundaries that overlap with Kabat CDRs. Other boundaries defining CDRs overlapping with the Kabat CDRs have been described by Padlan et al. (1995) FASEB J. 9: 133-139 and MacCallum (1996) J. Mol. Biol. 262(5): 732-745). Still other CDR boundary definitions may not strictly follow one of the above systems, but will nonetheless overlap with the Kabat CDRs, although they may be shortened or lengthened in light of prediction or experimental findings that particular residues or groups of residues or even entire CDRs do not significantly impact antigen binding. The methods used herein may utilize CDRs defined according to any of these systems, although certain embodiments use Kabat or Chothia defined CDRs.

The terms “Kabat numbering”, “Kabat definition” and “Kabat labeling” are used interchangeably herein. These terms refer to a system of numbering amino acid residues which are more variable (i.e., hypervariable) than other amino acid residues in the heavy and light chain variable regions of an antibody, or an antigen binding portion thereof (Kabat et al. (1971) Ann. NY Acad. Sci. 190: 382-391 and Kabat, E. et al. (1991) Sequences of Proteins of Immunological Interest, Fifth Edition, U.S. Department of Health and Human Services, NIH Publication No. 91-3242). For the heavy chain variable region, the hypervariable region ranges from amino acid positions 31 to 35 for CDR1, amino acid positions 50 to 65 for CDR2, and amino acid positions 95 to 102 for CDR3. For the light chain variable region, the hypervariable region ranges from amino acid positions 24 to 34 for CDR1, amino acid positions 50 to 56 for CDR2, and amino acid positions 89 to 97 for CDR3.

The growth and analysis of extensive public databases of amino acid sequences of variable heavy and light regions over the past twenty years have led to the understanding of the typical boundaries between framework regions (FR) and CDR sequences within variable region sequences and enabled persons skilled in this art to accurately determine the CDRs according to Kabat numbering, Chothia numbering, or other systems. See, e.g., Martin, “Protein Sequence and Structure Analysis of Antibody Variable Domains,” In Kontermann and Dübel, eds., Antibody Engineering (Springer-Verlag, Berlin, 2001), chapter 31, pages 432-433. A useful method of determining the amino acid sequences of Kabat CDRs within the amino acid sequences of variable heavy (VH) and variable light (VL) regions is provided below:

To identify a CDR-L1 amino acid sequence:
Starts approximately 24 amino acid residues from the amino terminus of the VL region;
Residue before the CDR-L1 sequence is always cysteine (C);
Residue after the CDR-L1 sequence is always a tryptophan (W) residue, typically Trp-Tyr-Gln (W-Y-Q), but also Trp-Leu-Gln (W-L-Q), Trp-Phe-Gln (W-F-Q), and Trp-Tyr-Leu (W-Y-L);
Length is typically 10 to 17 amino acid residues.

To identify a CDR-L2 amino acid sequence:
Starts always 16 residues after the end of CDR-L1;
Residues before the CDR-L2 sequence are generally Ile-Tyr (I-Y), but also Val-Tyr (V-Y), Ile-Lys (I-K), and Ile-Phe (I-F);
Length is always 7 amino acid residues.

To identify a CDR-L3 amino acid sequence:
Starts always 33 amino acids after the end of CDR-L2;
Residue before the CDR-L3 amino acid sequence is always a cysteine (C);
Residues after the CDR-L3 sequence are always Phe-Gly-X-Gly (F-G-X-G) (SEQ ID NO:7), where X is any amino acid;
Length is typically 7 to 11 amino acid residues.

To identify a CDR-H1 amino acid sequence:
Starts approximately 31 amino acid residues from amino terminus of VH region and always 9 residues after a cysteine (C);
Residues before the CDR-H1 sequence are always Cys-X-X-X-X-X-X-X-X

(SEQ ID NO:10), where X is any amino acid;
Residue after CDR-H1 sequence is always a Trp (W), typically Trp-Val (W-V), but also Trp-Ile (W-I), and Trp-Ala (W-A);
Length is typically 5 to 7 amino acid residues.

To identify a CDR-H2 amino acid sequence:
Starts always 15 amino acid residues after the end of CDR-H1;
Residues before CDR-H2 sequence are typically Leu-Glu-Trp-Ile-Gly (L-E-W-I-G) (SEQ ID NO:8), but other variations also;
Residues after CDR-H2 sequence are Lys/Arg-Leu/Ile/Val/Phe/Thr/Ala-Thr/Ser/Ile/Ala (K/R-L/I/V/F/T/A-T/S/I/A);
Length is typically 16 to 19 amino acid residues.

To identify a CDR-H3 amino acid sequence:
Starts always 33 amino acid residues after the end of CDR-H2 and always 3 after a cysteine (C)′
Residues before the CDR-H3 sequence are always Cys-X-X (C-X-X), where X is any amino acid, typically Cys-Ala-Arg (C-A-R);
Residues after the CDR-H3 sequence are always Trp-Gly-X-Gly (W-G-X-G) (SEQ ID NO:9), where X is any amino acid;
Length is typically 3 to 25 amino acid residues.

As used herein, the terms “acceptor” and “acceptor antibody” refer to the antibody or nucleic acid sequence providing or encoding at least 80%, at least 85%, at least 90%, at least 95%, at least 98%, or 100% of the amino acid sequences of one or more of the framework regions. In some embodiments, the term “acceptor” refers to the antibody amino acid or nucleic acid sequence providing or encoding the constant region(s). In yet another embodiment, the term “acceptor” refers to the antibody amino acid or nucleic acid sequence providing or encoding one or more of the framework regions and the constant region(s). In a specific embodiment, the term “acceptor” refers to a human antibody amino acid or nucleic acid sequence that provides or encodes at least 80%, at least 85%, at least 90%, at least 95%, at least 98%, or 100% of the amino acid sequences of one or more of the framework regions. In accordance with this embodiment, an acceptor may contain at least 1, at least 2, at least 3, least 4, at least 5, or at least 10 amino acid residues that does (do) not occur at one or more specific positions of a human antibody. An acceptor framework region and/or acceptor constant region(s) may be, e.g., derived or obtained from a germline antibody gene, a mature antibody gene, a functional antibody (e.g., antibodies well-known in the art, antibodies in development, or antibodies commercially available). As used herein, the term “canonical” residue refers to a residue in a CDR or framework that defines a particular canonical CDR structure as defined by Chothia et al. (1987) J. Mol. Biol. 196:901-917 and Chothia et al. (1992) J. Mol. Biol. 227:799-817). According to Chothia et al., critical portions of the CDRs of many antibodies have nearly identical peptide backbone confirmations despite great diversity at the level of amino acid sequence. Each canonical structure specifies primarily a set of peptide backbone torsion angles for a contiguous segment of amino acid residues forming a loop.

As used herein, the terms “donor” and “donor antibody” refer to an antibody providing one or more CDRs. In one embodiment, the donor antibody is an antibody from a species different from the antibody from which the framework regions are obtained or derived. In the context of a humanized antibody, the term “donor antibody” refers to a non-human antibody providing one or more CDRs. As used herein, the term “framework” or “framework sequence” refers to the remaining sequences of a variable region minus the CDRs. Because the exact definition of a CDR sequence can be determined by different systems, the meaning of a framework sequence is subject to correspondingly different interpretations. The six CDRs (CDR-L1, -L2, and -L3 of light chain and CDR-H1, -H2, and -H3 of heavy chain) also divide the framework regions on the light chain and the heavy chain into four sub-regions (FR1, FR2, FR3 and FR4) on each chain, in which CDR1 is positioned between FR1 and FR2, CDR2 between FR2 and FR3, and CDR3 between FR3 and FR4. Without specifying the particular sub-regions as FR1, FR2, FR3 or FR4, a framework region, as referred by others, represents the combined FR's within the variable region of a single, naturally occurring immunoglobulin chain. As used herein, a FR represents one of the four sub-regions, and FRs represents two or more of the four sub-regions constituting a framework region.

As used herein, the term “germline antibody gene” or “gene fragment” refers to an immunoglobulin sequence encoded by non-lymphoid cells that have not undergone the maturation process that leads to genetic rearrangement and mutation for expression of a particular immunoglobulin. (See, e.g., Shapiro et al., Crit. Rev. Immunol., 22(3): 183-200 (2002); Marchalonis et al., Adv. Exp. Med. Biol., 484: 13-30 (2001)). One of the advantages provided by various embodiments of the present invention stems from the recognition that germline antibody genes are more likely than mature antibody genes to conserve essential amino acid sequence structures characteristic of individuals in the species, hence less likely to be recognized as from a foreign source when used therapeutically in that species.

As used herein, the term “key” residues refer to certain residues within the variable region that have more impact on the binding specificity and/or affinity of an antibody, in particular a humanized antibody. A key residue includes, but is not limited to, one or more of the following: a residue that is adjacent to a CDR, a potential glycosylation site (can be either N- or O-glycosylation site), a rare residue, a residue capable of interacting with the antigen, a residue capable of interacting with a CDR, a canonical residue, a contact residue between heavy chain variable region and light chain variable region, a residue within the Vernier zone, and a residue in the region that overlaps between the Chothia definition of a variable heavy chain CDR1 and the Kabat definition of the first heavy chain framework.

The term “humanized antibody” refers to antibodies that comprise heavy and light chain variable region sequences from a non-human species (e.g., a mouse) but in which at least a portion of the VH and/or VL sequence has been altered to be more “human-like”, i.e., more similar to human germline variable sequences. One type of humanized antibody is a CDR-grafted antibody, in which human CDR sequences are introduced into non-human VH and VL sequences to replace the corresponding nonhuman CDR sequences. Also “humanized antibody” is an antibody or a variant, derivative, analog or fragment thereof which immunospecifically binds to an antigen of interest and which comprises a framework (FR) region having substantially the amino acid sequence of a human antibody and a complementary determining region (CDR) having substantially the amino acid sequence of a non-human antibody. As used herein, the term “substantially” in the context of a CDR refers to a CDR having an amino acid sequence at least 80%, at least 85%, at least 90%, at least 95%, at least 98% or at least 99% identical to the amino acid sequence of a non-human antibody CDR. A humanized antibody comprises substantially all of at least one, and typically two, variable domains (Fab, Fab′, F(ab′)₂, FabC, Fv) in which all or substantially all of the CDR regions correspond to those of a non-human immunoglobulin (i.e., donor antibody) and all or substantially all of the framework regions are those of a human immunoglobulin consensus sequence. In an embodiment, a humanized antibody also comprises at least a portion of an immunoglobulin constant region (Fc), typically that of a human immunoglobulin. In some embodiments, a humanized antibody contains both the light chain as well as at least the variable domain of a heavy chain. The antibody also may include the CH1, hinge, CH2, CH3, and CH4 regions of the heavy chain. In some embodiments, a humanized antibody only contains a humanized light chain. In some embodiments, a humanized antibody only contains a humanized heavy chain. In specific embodiments, a humanized antibody only contains a humanized variable domain of a light chain and/or humanized heavy chain.

A humanized antibody may be selected from any class of immunoglobulins, including IgM, IgG, IgD, IgA and IgE, and any isotype including without limitation IgG1, IgG2, IgG3, and IgG4. The humanized antibody may comprise sequences from more than one class or isotype, and particular constant domains may be selected to optimize desired effector functions using techniques well known in the art.

The framework and CDR regions of a humanized antibody need not correspond precisely to the parental sequences, e.g., the donor antibody CDR or the consensus framework may be mutagenized by substitution, insertion and/or deletion of at least one amino acid residue so that the CDR or framework residue at that site does not correspond to either the donor antibody or the consensus framework. In an exemplary embodiment, such mutations, however, will not be extensive. Usually, at least 80%, preferably at least 85%, more preferably at least 90%, and most preferably at least 95% of the humanized antibody residues will correspond to those of the parental FR and CDR sequences. As used herein, the term “consensus framework” refers to the framework region in the consensus immunoglobulin sequence. As used herein, the term “consensus immunoglobulin sequence” refers to the sequence formed from the most frequently occurring amino acids (or nucleotides) in a family of related immunoglobulin sequences (see, e.g., Winnaker, From Genes to Clones (Verlagsgesellschaft, Weinheim, Germany 1987)). In a family of immunoglobulins, each position in the consensus sequence is occupied by the amino acid occurring most frequently at that position in the family. If two amino acids occur equally frequently, either can be included in the consensus sequence.

With respect to constructing DVD-Ig or other binding protein molecules, a “linker” is used to denote a single amino acid or a polypeptide (“linker polypeptide”) comprising two or more amino acid residues joined by peptide bonds and used to link one or more antigen binding portions. Such linker polypeptides are well known in the art (see, e.g., Holliger et al., Proc. Natl. Acad. Sci. USA, 90: 6444-6448 (1993); Poljak, R. J., Structure, 2: 1121-1123 (1994)). Exemplary linkers include, but are not limited to, GGGGSG (SEQ ID NO:11), GGSGG (SEQ ID NO:12), GGGGSGGGGS (SEQ ID NO:13), GGSGGGGSG (SEQ ID NO:14), GGSGGGGSGS (SEQ ID NO:15), GGSGGGGSGGGGS (SEQ ID NO:16), GGGGSGGGGSGGGG (SEQ ID NO:17), GGGGSGGGGSGGGGS (SEQ ID NO:18), ASTKGP (SEQ ID NO:19), ASTKGPSVFPLAP (SEQ ID NO:20), TVAAP (SEQ ID NO:21), RTVAAP (SEQ ID NO:22), TVAAPSVFIFPP (SEQ ID NO:23), RTVAAPSVFIFPP (SEQ ID NO:24), AKTTPKLEEGEFSEAR (SEQ ID NO:25), AKTTPKLEEGEFSEARV (SEQ ID NO:26), AKTTPKLGG (SEQ ID NO:27), SAKTTPKLGG (SEQ ID NO:28), SAKTTP (SEQ ID NO:29), RADAAP (SEQ ID NO:30), RADAAPTVS (SEQ ID NO:31), RADAAAAGGPGS (SEQ ID NO:32), RADAAAAGGGGSGGGGSGGGGSGGGGS (SEQ ID NO:33), SAKTTPKLEEGEFSEARV (SEQ ID NO:34), ADAAP (SEQ ID NO:35), ADAAPTVSIFPP (SEQ ID NO:36), QPKAAP (SEQ ID NO:37), QPKAAPSVTLFPP (SEQ ID NO:38), AKTTPP (SEQ ID NO:39), AKTTPPSVTPLAP (SEQ ID NO:40), AKTTAP (SEQ ID NO:41), AKTTAPSVYPLAP (SEQ ID NO:42), GENKVEYAPALMALS (SEQ ID NO:43), GPAKELTPLKEAKVS (SEQ ID NO:44), and GHEAAAVMQVQYPAS (SEQ ID NO:45).

As used herein, “Vernier” zone refers to a subset of framework residues that may adjust CDR structure and fine-tune the fit to antigen as described by Foote and Winter, J. Mol. Biol., 224:487-499 (1992), which is incorporated herein by reference). Vernier zone residues form a layer underlying the CDRs and may impact on the structure of CDRs and the affinity of the antibody.

As used herein, the term “neutralizing” refers to neutralization of the biological activity of an antigen (e.g., the cytokines IL-1α and IL-1β) when a binding protein specifically binds the antigen. Preferably, a neutralizing binding protein described herein binds to h IL-1β resulting in the inhibition of a biological activity of hIL-1β. Preferably, the neutralizing binding protein binds h IL-1β and reduces a biologically activity of hIL-1β by at least about 20%, 40%, 60%, 80%, 85%, or more Inhibition of a biological activity of h IL-1β by a neutralizing binding protein can be assessed by measuring one or more indicators of h IL-1β biological activity well known in the art. For example inhibition of human IL-6 secretion by IL-1β induction in HS27 cells.

The term “activity” includes activities such as the binding specificity/affinity of an antibody for an antigen, for example, an anti-h IL-1β antibody that binds to an IL-1β antigen and/or the neutralizing potency of an antibody, for example, an anti-IL-1β antibody whose binding to h IL-1β inhibits the biological activity of h IL-1β, for example, inhibition of human IL-6 secretion by IL-1β induction in HS27 cells.

The term “epitope” includes any polypeptide determinant capable of specific binding to an immunoglobulin or T-cell receptor. In certain embodiments, epitope determinants include chemically active surface groupings of molecules such as amino acids, sugar side chains, phosphoryl, or sulfonyl, and, in certain embodiments, may have specific three dimensional structural characteristics, and/or specific charge characteristics. An epitope is a region of an antigen that is bound by an antibody. In certain embodiments, an antibody is said to specifically bind an antigen when it preferentially recognizes its target antigen in a complex mixture of proteins and/or macromolecules. Antibodies are said to “bind to the same epitope” if the antibodies cross-compete (one prevents the binding or modulating effect of the other). In addition, structural definitions of epitopes (overlapping, similar, identical) are informative, but functional definitions are often more relevant as they encompass structural (binding) and functional (modulation, competition) parameters.

The term “surface plasmon resonance”, as used herein, refers to an optical phenomenon that allows for the analysis of real-time biospecific interactions by detection of alterations in protein concentrations within a biosensor matrix, for example using the BIAcore system (Pharmacia Biosensor AB, Uppsala, Sweden and Piscataway, N.J.). For further descriptions, see Jönsson et al., Ann. Biol. Clin., 51: 19-26 (1993); Jönsson et al., BioTechniques, 11: 620-627 (1991); Johnsson et al., J. Mol. Recognit., 8: 125-131 (1995); and Johnsson et al., Anal. Biochem., 198: 268-277 (1991).

The term “Kₒₙ” (also “Kon”, “kon”), as used herein, is intended to refer to the on rate constant for association of a binding protein (e.g., an antibody) to an antigen to form an association complex, e.g., antibody/antigen complex, as is known in the art. The “Kₒₙ” also is known by the terms “association rate constant”, or “ka”, as used interchangeably herein. This value indicates the binding rate of an antibody to its target antigen or the rate of complex formation between an antibody and antigen as is shown by the equation below:

Antibody (“Ab”)+Antigen (“Ag”)→Ab-Ag.

The term “K_{off}” (also “Koff”, “koff”), as used herein, is intended to refer to the off rate constant for dissociation, or “dissociation rate constant”, of a binding protein (e.g., an antibody) from an association complex (e.g., an antibody/antigen complex) as is known in the art. This value indicates the dissociation rate of an antibody from its target antigen or separation of Ab-Ag complex over time into free antibody and antigen as shown by the equation below:

Ab+Ag←Ab-Ag.

The term “K_{D}” (also “K_{d}”), as used herein, is intended to refer to the “equilibrium dissociation constant”, and refers to the value obtained in a titration measurement at equilibrium, or by dividing the dissociation rate constant (Koff) by the association rate constant (Kon). The association rate constant (Kon), the dissociation rate constant (Koff), and the equilibrium dissociation constant (K are used to represent the binding affinity of an antibody to an antigen. Methods for determining association and dissociation rate constants are well known in the art. Using fluorescence-based techniques offers high sensitivity and the ability to examine samples in physiological buffers at equilibrium. Other experimental approaches and instruments such as a BIAcore® (biomolecular interaction analysis) assay can be used (e.g., instrument available from BIAcore International AB, a GE Healthcare company, Uppsala, Sweden). Additionally, a KinExA® (Kinetic Exclusion Assay) assay, available from Sapidyne Instruments (Boise, Id.) can also be used.

The term “AUC” or “area under the curve” is related to clearance. A higher clearance rate is related to a smaller AUC, and a lower clearance rate is related to a larger AUC value. The AUC higher values represent slower clearance rates.

As used herein, the term “volume of distribution” is a term used to quantify the distribution of a drug, e.g., an anti-IL-I α/β dual variable domain immunoglobulin, or antigen-binding portion thereof, between plasma and the rest of the body after dosing. The volume of distribution is the theoretical volume in which the total amount of drug would need to be uniformly distributed in order to produce the desired blood concentration of the drug.

The term “half-life” of (T½) as used herein is a term used to quantify the time taken for half the dose of a drug to be excreted by a subject.

The term “Cmax” as used herein is a term used to quantify to the maximum or peak serum or plasma concentration of an agent observed in a subject after its administration.

The term “bioavailability” or “F” as used herein refers to a fraction or percent of a dose which is absorbed and enters the systemic circulation after administration of a given dosage form. See international publication number WO2013078135 published May 30, 2013, which is incorporated by reference herein in its entirety.

The terms “label” and “detectable label” mean a moiety attached to a specific binding partner, such as an antibody or an analyte, e.g., to render the reaction between members of a specific binding pair, such as an antibody and an analyte, detectable. The specific binding partner, e.g., antibody or analyte, so labeled is referred to as “detectably labeled”. Thus, the term “labeled binding protein” as used herein, refers to a protein with a label incorporated that provides for the identification of the binding protein. In an embodiment, the label is a detectable marker that can produce a signal that is detectable by visual or instrumental means, e.g., incorporation of a radiolabeled amino acid or attachment to a polypeptide of biotinyl moieties that can be detected by marked avidin or streptavidin (e.g., streptavidin containing a fluorescent marker or enzymatic activity that can be detected by optical or colorimetric methods). Examples of labels for polypeptides include, but are not limited to, the following: radioisotopes or radionuclides (e.g., ³H, ¹⁴C, ³⁵S, ⁹⁰Y, ⁹⁹Tc, ¹¹¹In, ¹²⁵I, ¹³¹I, ¹⁷⁷Lu, ¹⁶⁶Ho, or ¹⁵³Sm), chromogens, fluorescent labels (e.g., FITC, rhodamine, lanthanide phosphors), enzymatic labels (e.g., horseradish peroxidase, luciferase, alkaline phosphatase), chemiluminescent markers, biotinyl groups, predetermined polypeptide epitopes recognized by a secondary reporter (e.g., leucine zipper pair sequences, binding sites for secondary antibodies, metal binding domains, epitope tags), and magnetic agents (e.g., gadolinium chelates). Representative examples of labels commonly employed for immunoassays include moieties that produce light, e.g., acridinium compounds, and moieties that produce fluorescence, e.g., fluorescein. Other labels are described herein. In this regard, the moiety itself may not be detectably labeled but may become detectable upon reaction with yet another moiety. Use of the term “detectably labeled” is intended to encompass the latter type of detectable labeling.

The term “IL-1α binding protein conjugate” refers to an IL-1α binding protein described herein chemically linked to a second chemical moiety, such as a therapeutic or cytotoxic agent.

The term “IL-1β binding protein conjugate” refers to an IL-1β binding protein described herein chemically linked to a second chemical moiety, such as a therapeutic or cytotoxic agent. The term “agent” is used herein to denote a chemical compound, a mixture of chemical compounds, a biological macromolecule, or an extract made from biological materials. Preferably the therapeutic or cytotoxic agents include, but are not limited to, pertussis toxin, taxol, cytochalasin B, gramicidin D, ethidium bromide, emetine, mitomycin, etoposide, tenoposide, vincristine, vinblastine, colchicine, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. When employed in the context of an immunoassay, an IL-1β binding protein conjugate may be a detectably labeled antibody, which is used as the detection antibody.

The terms “crystal” and “crystallized” as used herein, refer to a binding protein (e.g., an antibody), or antigen binding portion thereof, that exists in the form of a crystal. Crystals are one form of the solid state of matter that is distinct from other forms such as the amorphous solid state or the liquid crystalline state. Crystals are composed of regular, repeating, three-dimensional arrays of atoms, ions, molecules (e.g., proteins such as antibodies), or molecular assemblies (e.g., antigen/antibody complexes). These three-dimensional arrays are arranged according to specific mathematical relationships that are well-understood in the field. The fundamental unit, or building block, that is repeated in a crystal is called the asymmetric unit. Repetition of the asymmetric unit in an arrangement that conforms to a given, well-defined crystallographic symmetry provides the “unit cell” of the crystal. Repetition of the unit cell by regular translations in all three dimensions provides the crystal. See Giegé et al., Chapter 1, In Crystallization of Nucleic Acids and Proteins, a Practical Approach, 2nd ed., (Ducruix and Giegé, eds.) (Oxford University Press, New York, 1999) pp. 1-16.

The term “polynucleotide” means a polymeric form of two or more nucleotides, either ribonucleotides or deoxynucleotides or a modified form of either type of nucleotide. The term includes single and double stranded forms of DNA.

The term “isolated polynucleotide” shall mean a polynucleotide (e.g., of genomic, cDNA, or synthetic origin, or some combination thereof) that, by virtue of its origin, the “isolated polynucleotide” is not associated with all or a portion of a polynucleotide with which the “isolated polynucleotide” is found in nature; is operably linked to a polynucleotide that it is not linked to in nature; or does not occur in nature as part of a larger sequence.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the invention is intended to include such other forms of expression vectors, such as viral vectors (e.g., replication defective retroviruses, adenoviruses and adeno-associated viruses), which serve equivalent functions.

The term “operably linked” refers to a juxtaposition wherein the components described are in a relationship permitting them to function in their intended manner. A control sequence “operably linked” to a coding sequence is ligated in such a way that expression of the coding sequence is achieved under conditions compatible with the control sequences. “Operably linked” sequences include both expression control sequences that are contiguous with the gene of interest and expression control sequences that act in trans or at a distance to control the gene of interest. The term “expression control sequence” as used herein refers to polynucleotide sequences that are necessary to effect the expression and processing of coding sequences to which they are ligated. Expression control sequences include appropriate transcription initiation, termination, promoter and enhancer sequences; efficient RNA processing signals such as splicing and polyadenylation signals; sequences that stabilize cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include components whose presence is essential for expression and processing, and can also include additional components whose presence is advantageous, for example, leader sequences and fusion partner sequences.

“Transformation”, as defined herein, refers to any process by which exogenous DNA enters a host cell. Transformation may occur under natural or artificial conditions using various methods well known in the art. Transformation may rely on any known method for the insertion of foreign nucleic acid sequences into a prokaryotic or eukaryotic host cell. The method is selected based on the host cell being transformed and may include, but is not limited to, viral infection, electroporation, lipofection, and particle bombardment. Such “transformed” cells include stably transformed cells in which the inserted DNA is capable of replication either as an autonomously replicating plasmid or as part of the host chromosome. They also include cells which transiently express the inserted DNA or RNA for limited periods of time.

The term “recombinant host cell” (or simply “host cell”), is intended to refer to a cell into which exogenous DNA has been introduced. In an embodiment, the host cell comprises two or more (e.g., multiple) nucleic acids encoding antibodies, such as the host cells described in U.S. Pat. No. 7,262,028, for example. Such terms are intended to refer not only to the particular subject cell, but also to the progeny of such a cell. Because certain modifications may occur in succeeding generations due to either mutation or environmental influences, such progeny may not, in fact, be identical to the parent cell, but are still included within the scope of the term “host cell” as used herein. In an embodiment, host cells include prokaryotic and eukaryotic cells selected from any of the Kingdoms of life. In another embodiment, eukaryotic cells include protist, fungal, plant and animal cells. In another embodiment, host cells include but are not limited to the prokaryotic cell line Escherichia coli; mammalian cell lines CHO, HEK 293, COS, NSO, SP2 and PER.C6; the insect cell line Sf9; and the fungal cell Saccharomyces cerevisiae.

Standard techniques may be used for recombinant DNA, oligonucleotide synthesis, and tissue culture and transformation (e.g., electroporation, lipofection). Enzymatic reactions and purification techniques may be performed according to manufacturer's specifications or as commonly accomplished in the art or as described herein. The foregoing techniques and procedures may be generally performed according to conventional methods well known in the art and as described in various general and more specific references that are cited and discussed throughout the present specification. See e.g., Sambrook et al., Molecular Cloning: A Laboratory Manual, 2nd ed. (Cold Spring Harbor Laboratory Press, Cold Spring Harbor, N.Y., 1989).

“Transgenic organism”, as known in the art, refers to an organism having cells that contain a transgene, wherein the transgene introduced into the organism (or an ancestor of the organism) expresses a polypeptide not naturally expressed in the organism. A “transgene” is a DNA construct, which is stably and operably integrated into the genome of a cell from which a transgenic organism develops, directing the expression of an encoded gene product in one or more cell types or tissues of the transgenic organism.

The terms “regulate” and “modulate” are used interchangeably, and, as used herein, refers to a change or an alteration in the activity of a molecule of interest (e.g., the biological activity of human IL-1α or human IL-1β). Modulation may be an increase or a decrease in the magnitude of a certain activity or function of the molecule of interest. Exemplary activities and functions of a molecule include, but are not limited to, binding characteristics, enzymatic activity, cell receptor activation, and signal transduction.

Correspondingly, the term “modulator,” as used herein, is a compound capable of changing or altering an activity or function of a molecule of interest (e.g., the biological activity of hIL-1β). For example, a modulator may cause an increase or decrease in the magnitude of a certain activity or function of a molecule compared to the magnitude of the activity or function observed in the absence of the modulator. In certain embodiments, a modulator is an inhibitor, which decreases the magnitude of at least one activity or function of a molecule. Exemplary inhibitors include, but are not limited to, proteins, peptides, antibodies, peptibodies, carbohydrates or small organic molecules. Peptibodies are described, e.g., in PCT Publication No. WO 01/83525.

The term “agonist”, as used herein, refers to a modulator that, when contacted with a molecule of interest, causes an increase in the magnitude of a certain activity or function of the molecule compared to the magnitude of the activity or function observed in the absence of the agonist. Particular agonists of interest may include, but are not limited to, IL-1β polypeptides, nucleic acids, carbohydrates, or any other molecule that binds to hIL-1β.

The terms “antagonist” and “inhibitor”, as used herein, refer to a modulator that, when contacted with a molecule of interest causes a decrease in the magnitude of a certain activity or function of the molecule compared to the magnitude of the activity or function observed in the absence of the antagonist. Particular antagonists of interest include those that block or modulate the biological or immunological activity of human IL-1β. Antagonists and inhibitors of human IL-1β may include, but are not limited to, proteins, nucleic acids, carbohydrates, or any other molecules, which bind to human IL-1β.

As used herein, the term “effective amount” refers to the amount of a therapy that is sufficient to reduce or ameliorate the severity and/or duration of a disorder or one or more symptoms thereof; prevent the advancement of a disorder; cause regression of a disorder; prevent the recurrence, development, onset, or progression of one or more symptoms associated with a disorder; detect a disorder; or enhance or improve the prophylactic or therapeutic effect(s) of another therapy (e.g., prophylactic or therapeutic agent).

“Patient” and “subject” may be used interchangeably herein to refer to an animal, such as a mammal, including a primate (for example, a human, a monkey, and a chimpanzee), a non-primate (for example, a cow, a pig, a camel, a llama, a horse, a goat, a rabbit, a sheep, a hamster, a guinea pig, a cat, a dog, a rat, a mouse, a whale), a bird (e.g., a duck or a goose), and a shark. Preferably, a patient or subject is a human, such as a human being treated or assessed for a disease, disorder or condition, a human at risk for a disease, disorder or condition, a human having a disease, disorder or condition, and/or human being treated for a disease, disorder or condition.

The term “sample”, as used herein, is used in its broadest sense. A “biological sample”, as used herein, includes, but is not limited to, any quantity of a substance from a living thing or formerly living thing. Such living things include, but are not limited to, humans, non-human primates, mice, rats, monkeys, dogs, rabbits and other animals. Such substances include, but are not limited to, blood (e.g., whole blood), plasma, serum, urine, amniotic fluid, synovial fluid, endothelial cells, leukocytes, monocytes, other cells, organs, tissues, bone marrow, lymph nodes and spleen.

“Component”, “components,” and “at least one component,” refer generally to a capture antibody, a detection or conjugate antibody, a control, a calibrator, a series of calibrators, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample, such as a patient urine, serum or plasma sample, in accordance with the methods described herein and other methods known in the art. Thus, in the context of the present disclosure, “at least one component,” “component,” and “components” can include a polypeptide or other analyte as above, such as a composition comprising an analyte such as polypeptide, which is optionally immobilized on a solid support, such as by binding to an anti-analyte (e.g., anti-polypeptide) antibody. Some components can be in solution or lyophilized for reconstitution for use in an assay.

“Control” refers to a composition known to not analyte (“negative control”) or to contain analyte (“positive control”). A positive control can comprise a known concentration of analyte. “Control,” “positive control,” and “calibrator” may be used interchangeably herein to refer to a composition comprising a known concentration of analyte. A “positive control” can be used to establish assay performance characteristics and is a useful indicator of the integrity of reagents (e.g., analytes).

“Predetermined cutoff” and “predetermined level” refer generally to an assay cutoff value that is used to assess diagnostic/prognostic/therapeutic efficacy results by comparing the assay results against the predetermined cutoff/level, where the predetermined cutoff/level already has been linked or associated with various clinical parameters (e.g., severity of disease, progression/nonprogression/improvement, etc.). While the present disclosure may provide exemplary predetermined levels, it is well-known that cutoff values may vary depending on the nature of the immunoassay (e.g., antibodies employed, etc.). It further is well within the ordinary skill of one in the art to adapt the disclosure herein for other immunoassays to obtain immunoassay-specific cutoff values for those other immunoassays based on this disclosure. Whereas the precise value of the predetermined cutoff/level may vary between assays, correlations as described herein (if any) should be generally applicable.

“Pretreatment reagent,” e.g., lysis, precipitation and/or solubilization reagent, as used in a diagnostic assay as described herein is one that lyses any cells and/or solubilizes any analyte that is/are present in a test sample. Pretreatment is not necessary for all samples, as described further herein. Among other things, solubilizing the analyte (e.g., polypeptide of interest) may entail release of the analyte from any endogenous binding proteins present in the sample. A pretreatment reagent may be homogeneous (not requiring a separation step) or heterogeneous (requiring a separation step). With use of a heterogeneous pretreatment reagent there is removal of any precipitated analyte binding proteins from the test sample prior to proceeding to the next step of the assay.

“Quality control reagents” in the context of immunoassays and kits described herein, include, but are not limited to, calibrators, controls, and sensitivity panels. A “calibrator” or “standard” typically is used (e.g., one or more, such as a plurality) in order to establish calibration (standard) curves for interpolation of the concentration of an analyte, such as an antibody or an analyte. Alternatively, a single calibrator, which is near a predetermined positive/negative cutoff, can be used. Multiple calibrators (i.e., more than one calibrator or a varying amount of calibrator(s)) can be used in conjunction so as to comprise a “sensitivity panel.”

“Risk” refers to the possibility or probability of a particular event occurring either presently or at some point in the future. “Risk stratification” refers to an array of known clinical risk factors that allows physicians to classify patients into a low, moderate, high or highest risk of developing a particular disease, disorder or condition.

“Specific” and “specificity” in the context of an interaction between members of a specific binding pair (e.g., an antigen (or fragment thereof) and an antibody (or antigenically reactive fragment thereof)) refer to the selective reactivity of the interaction. The phrase “specifically binds to” and analogous phrases refer to the ability of antibodies (or antigenically reactive fragments thereof) to bind specifically to analyte (or a fragment thereof) and not bind specifically to other entities.

“Specific binding partner” is a member of a specific binding pair. A specific binding pair comprises two different molecules, which specifically bind to each other through chemical or physical means. Therefore, in addition to antigen and antibody specific binding pairs of common immunoassays, other specific binding pairs can include biotin and avidin (or streptavidin), carbohydrates and lectins, complementary nucleotide sequences, effector and receptor molecules, cofactors and enzymes, enzyme inhibitors and enzymes, and the like. Furthermore, specific binding pairs can include members that are analogs of the original specific binding members, for example, an analyte-analog Immunoreactive specific binding members include antigens, antigen fragments, and antibodies, including monoclonal and polyclonal antibodies as well as complexes, fragments, and variants (including fragments of variants) thereof, whether isolated or recombinantly produced.

“Variant” as used herein means a polypeptide that differs from a given polypeptide (e.g., IL-1β, BNP, NGAL, or HIV polypeptide, or anti-polypeptide antibody) in amino acid sequence by the addition (e.g., insertion), deletion, or conservative substitution of amino acids, but that retains the biological activity of the given polypeptide (e.g., a variant IL-1β can compete with anti-IL-1β antibody for binding to IL-1β). A conservative substitution of an amino acid, i.e., replacing an amino acid with a different amino acid of similar properties (e.g., hydrophilicity and degree and distribution of charged regions) is recognized in the art as typically involving a minor change. These minor changes can be identified, in part, by considering the hydropathic index of amino acids, as understood in the art (see, e.g., Kyte et al., J. Mol. Biol., 157: 105-132 (1982)). The hydropathic index of an amino acid is based on a consideration of its hydrophobicity and charge. It is known in the art that amino acids of similar hydropathic indexes can be substituted and still retain protein function. In one aspect, amino acids having hydropathic indexes of ±2 are substituted. The hydrophilicity of amino acids also can be used to reveal substitutions that would result in proteins retaining biological function. A consideration of the hydrophilicity of amino acids in the context of a peptide permits calculation of the greatest local average hydrophilicity of that peptide, a useful measure that has been reported to correlate well with antigenicity and immunogenicity (see, e.g., U.S. Pat. No. 4,554,101). Substitution of amino acids having similar hydrophilicity values can result in peptides retaining biological activity, for example immunogenicity, as is understood in the art. In one aspect, substitutions are performed with amino acids having hydrophilicity values within ±2 of each other. Both the hydrophobicity index and the hydrophilicity value of amino acids are influenced by the particular side chain of that amino acid. Consistent with that observation, amino acid substitutions that are compatible with biological function are understood to depend on the relative similarity of the amino acids, and particularly the side chains of those amino acids, as revealed by the hydrophobicity, hydrophilicity, charge, size, and other properties. “Variant” also can be used to describe a polypeptide or fragment thereof that has been differentially processed, such as by proteolysis, phosphorylation, or other post-translational modification, yet retains its biological activity or antigen reactivity, e.g., the ability to bind to IL-1β. Use of “variant” herein is intended to encompass fragments of a variant unless otherwise contradicted by context.

A number of abbreviations are used herein to describe aspects of the invention. Below is a list of commonly used abbreviations.
ACR—American College of Rheumatology
ADA—Anti-drug antibody
AE—Adverse event
ALT—Alanine aminotransferase
ANC—Absolute neutrophil count
AUC—Area under the serum concentration-time curve; e.g., (μg·hr/mL or mg·hr/mL)
BA—Bioavailability
BQL—Below quantitation limit
BUN—Blood Urea Nitrogen
C1/F—Apparent clearance
C1M—Matrix metalloproteinase-mediated degradation of type I collagen
C2M—Matrix metalloproteinase-mediated degradation of type II collagen
C3M—Matrix metalloproteinase-mediated degradation of type III collagen
CD—Crohn's disease
CDAI—Clinical Disease Activity Index
CHSO—50% hemolytic complement activity (assay)
CIA—Collagen-induced arthritis
CIC—Circulating immune complex
Cmax—Maximum observed serum concentration
COX—Cyclooxygenase
CR—Clinical Remission
CRPM—Matrix metalloproteinase-mediated C-reactive protein
Ctrough—Trough concentration; lowest concentration of the drug in the blood that is measured after a dose
CTX-I—C-terminal telopeptide type I collagen
CTX-II—C-terminal telopeptide type II collagen
DAS-28—Disease activity score 28
DB—Double-blind
DR—Disease Response
DVD-Ig™—Dual-variable domain immunoglobulin
ECG—Electrocardiogram
eCRF—Electronic case report form
ED50—Dose required to produce a 50% reduction in response
EDC—Electronic data capture
ELISA—Enzyme-linked immunosorbent assay
EOW—Every other week
ESRB—External Safety Review Board
EULAR—European League against Rheumatism
EW—Every Week
F—Bioavailability
FACIT-F—Functional Assessment of Chronic Illness Therapy-Fatigue
FIH—First-in-human
FITC—Fluorescein isothiocyanate
GCP—Good Clinical Practice
GLP—Good Laboratory Practice
HAQ-DI—Health Assessment Questionnaire Disability Index
Hrs—Hours
hsCRP—High sensitivity C-reactive protein
IC50—Inhibitory concentration 50 percent
ICH—International Conference on Harmonisation
IEC—Independent Ethics Committee
IgG—Immunoglobulin G
IgG1—Immunoglobulin G1
IHC—Immunohistochemical
IL—Interleukin
IL-17—Interleukin 17
IP—Intraperitoneal
IRB—Institutional Review Board
IUD—Intrauterine Device
IV—Intravenous(ly)
IVRS—Interactive voice response system
IWRS—Interactive web response system
JAK—Janus kinase
KC—Keratinocyte-derived chemokine
KD—Dissociation constant
LDA—Low Disease Activity
mAb—Monoclonal antibody
MAD—Multiple ascending dose
MAS—Mean arthritic score
MedDRA—Medical Dictionary for Regulatory Activities
mg/kg—Milligrams per kilogram
micro-CT—Micro-computed tomography
MMP—Matrix metalloproteinases
MMP-3—Matrix metalloproteinase 3
MRNA—Messenger ribonucleic acid
MRT—Mean residence time
MSD—Meso Scale Discovery
NA—Not applicable
NOAEL—No-observed-adverse-effect-level
NSAID—Nonsteroidal anti-inflammatory drugs
OLE—Open-Label Extension
PD—Premature Discontinuation or Pharmacodynamic
PDR—Post-dose reaction
PEF—Peak Expiratory Flow
PGA—Physician's Global Assessment of Disease Activity
PK—Pharmacokinetic(s)
PT—Preferred term
PtGA—Patient's Global Assessment of Disease Activity
RA—Rheumatoid arthritisRA-WIS—Rheumatoid Arthritis Work Instability Scale
RBC—Red blood cells
RCT—Randomized Controlled Trial
rIL-17—Recombinant interleukin-17
rTNF—Recombinant tumor necrosis factor
SAD—Single ascending dose
SAE—Serious adverse event
SC—Subcutaneous(ly)
SCR—Screening
SD—Standard deviation
SF-36v2—Short form health surveySGPT/ALT—Serum glutamic-pyruvic transaminase
SGOT/AST—Serum glutamic-oxaloacetic transaminase
SJC—Swollen joint count
SOC—System organ class
SUSAR—Suspected unexplained serious adverse reaction
TB—Tuberculosis
TJC—Tender joint count
Tmax—Time to reach maximum concentration
TNF—Tumor necrosis factor
t½ —Terminal phase elimination half-life
μg/mL—Micrograms per milliliter
ULN—Upper limit of normal
VAS—Visual analog scale
VICM—Citrullinated and matrix metalloproteinase—degraded vimentin
Vss—Volume of distribution
Vss/F—Volume of distribution at steady-state
WBC—White blood cell

### A. Anti-IL-1α and Anti-IL-1β DVD-Ig™ Binding Proteins

A multivalent multispecific dual variable domain immunoglobulin (DVD-Ig™) binding protein is designed such that two different light chain variable domains (VL) from two different parent monoclonal antibodies are linked in tandem directly or via a short linker by recombinant DNA techniques, followed by the light chain constant domain. Similarly, the heavy chain comprises two different heavy chain variable domains (VH) linked in tandem, followed by the constant domain CH1 and Fc region.

In certain aspects, the methods of the invention employ dual variable domain immunoglobulin binding proteins (DVD-Igs) that bind one or more epitopes of IL-1α and IL-1β. An exemplary embodiment of such DVD-Ig molecules comprises a heavy chain that comprises the structural formula VD1-(X1)n-VD2-C-(X2)n, wherein VD1 is a first heavy chain variable domain, VD2 is a second heavy chain variable domain, C is a heavy chain constant domain, X1 is a linker with the proviso that it is not CH1, X2 is an Fc region, and n is 0 or 1, and preferably 1; and a light chain that comprises the structural formula VD1-(X1)n-VD2-C-(X2)n, wherein VD1 is a first light chain variable domain, VD2 is a second light chain variable domain, C is a light chain constant domain, X1 is a linker with the proviso that it is not CH1, and X2 does not comprise an Fc region; and n is 0 or 1, and preferably 1. Such a DVD-Ig may comprise two such heavy chains and two such light chains, wherein each chain comprises variable domains linked in tandem without an intervening constant region between variable regions, wherein a heavy chain and a light chain associate to form two tandem antigen binding sites, and a pair of heavy and light chains may associate with another pair of heavy and light chains to form a tetrameric binding protein with four antigen binding sites. In another embodiment, a DVD-Ig molecule may comprise heavy and light chains that each comprise three variable domains, e.g., VD1, VD2, VD3, linked in tandem without an intervening constant region between variable domains, wherein a pair of heavy and light chains may associate to form three antigen binding sites, and wherein a pair of heavy and light chains may associate with another pair of heavy and light chains to form a tetrameric binding protein with six antigen binding sites.

The linker sequence may be a single amino acid or a linker polypeptide comprising two or more amino acid residues joined by peptide bonds. In an embodiment, a linker sequence is selected from the group consisting of GGGGSG (SEQ ID NO:11), GGSGG (SEQ ID NO:12), GGGGSGGGGS (SEQ ID NO:13), GGSGGGGSG (SEQ ID NO:14), GGSGGGGSGS (SEQ ID NO:15), GGSGGGGSGGGGS (SEQ ID NO:16), GGGGSGGGGSGGGG (SEQ ID NO:17), GGGGSGGGGSGGGGS (SEQ ID NO:18), ASTKGP (SEQ ID NO:19), ASTKGPSVFPLAP (SEQ ID NO:20), TVAAP (SEQ ID NO:21), RTVAAP (SEQ ID NO:22), TVAAPSVFIFPP (SEQ ID NO:23), RTVAAPSVFIFPP (SEQ ID NO:24), AKTTPKLEEGEFSEAR (SEQ ID NO:25), AKTTPKLEEGEFSEARV (SEQ ID NO:26), AKTTPKLGG (SEQ ID NO:27), SAKTTPKLGG (SEQ ID NO:28), SAKTTP (SEQ ID NO:29), RADAAP (SEQ ID NO:30), RADAAPTVS (SEQ ID NO:31), RADAAAAGGPGS (SEQ ID NO:32), RADAAAAGGGGSGGGGSGGGGSGGGGS (SEQ ID NO:33), SAKTTPKLEEGEFSEARV (SEQ ID NO:34), ADAAP (SEQ ID NO:35), ADAAPTVSIFPP (SEQ ID NO:36), QPKAAP (SEQ ID NO:37), QPKAAPSVTLFPP (SEQ ID NO:38), AKTTPP (SEQ ID NO:39), AKTTPPSVTPLAP (SEQ ID NO:40), AKTTAP (SEQ ID NO:41), AKTTAPSVYPLAP (SEQ ID NO:42), GENKVEYAPALMALS (SEQ ID NO:43), GPAKELTPLKEAKVS (SEQ ID NO:44), and GHEAAAVMQVQYPAS (SEQ ID NO:45).

The choice of linker sequences is based on crystal structure analysis of several Fab molecules. There is a natural flexible linkage between the variable domain and the CH1/CL constant domain in Fab or antibody molecular structure. This natural linkage comprises approximately 10-12 amino acid residues, contributed by 4-6 residues from C-terminus of V domain and 4-6 residues from the N-terminus of CL/CH1 domain. DVD-Igs described herein can be generated using N-terminal 5-6 amino acid residues, or 11-12 amino acid residues, of CL or CH1 as linker in light chain and heavy chain of DVD-Ig, respectively. The N-terminal residues of CL or CH1 domains, particularly the first 5-6 amino acid residues, adopt a loop conformation without strong secondary structures, and therefore can act as flexible linkers between the two variable domains. The N-terminal residues of CL or CH1 domains are natural extension of the variable domains, as they are part of the Ig sequences, and therefore minimize to a large extent any immunogenicity potentially arising from the linkers and junctions.

Other linker sequences may include any sequence of any length of CL/CH1 domain but not all residues of CL/CH1 domain; for example the first 5-12 amino acid residues of the CL/CH1 domains; the light chain linkers can be from Cκ or Cλ; and the heavy chain linkers can be derived from CH1 of any isotypes, including Cγ1, Cγ2, Cγ3, Cγ4, Cα1, Cα2, Cδ, Cε, and Cμ. Linker sequences may also be derived from other proteins such as Ig-like proteins, (e.g., TCR, FcR, KIR); G/S based sequences; hinge region-derived sequences; and other natural sequences from other proteins.

In an embodiment a constant domain is linked to the two linked variable domains using recombinant DNA techniques. In an embodiment, a sequence comprising tandemly linked heavy chain variable domains is linked to a heavy chain constant domain and a sequence comprising tandemly linked light chain variable domains is linked to a light chain constant domain. In an embodiment, the constant domains are human heavy chain constant domain and human light chain constant domain, respectively. In an embodiment, the DVD heavy chain is further linked to an Fc region. The Fc region may be a native sequence Fc region, or a variant Fc region. In another embodiment, the Fc region is a human Fc region. In another embodiment the Fc region includes Fc region from IgG1, IgG2, IgG3, IgG4, IgA, IgM, IgE, or IgD.

In a most preferred embodiment, two heavy chain DVD polypeptides and two light chain DVD polypeptides are combined to form a DVD-Ig molecule. Exemplary amino acid sequences of heavy and light chains of DVD-Ig proteins capable of binding human IL-1β and human IL-1α are set forth in Table 1. In Table 3, the amino acid sequences for the E26.13 and E26.35 VL regions are designated SEQ ID NO:62 and SEQ ID NO:92, respectively, instead of SEQ ID NO:136 and SEQ ID NO:137, to account for the inclusion of a C-terminal arginine (R) residue.

| | | |
|---|---|---|
| E26.13 | SEQ ID | DIQMTQSPSSLSASVGDRVTITCRASGNIH |
| VL | NO: | NYLTWYQQTPGKAPKLLIYNAKTLADGVPS |
| | 136 | RFSGSGSGTDYTFTISSLQPEDIATYYCQH |
| | | FWSIPYTFGQGTKLQIT |
| E26.35 | SEQ ID | DIQMTQSPSSLSASVGDRVTITCRASGNIH |
| VL | NO: | NYLTWYQQTPGKAPKLLIYNAKTLADGVPS |
| | 137 | RFSGSGSGTDYTFTISSLQPEDIATYYCQH |
| | | FWSIPYTFGQGTKLQIT |

This C-terminal arginine residue is understood by those skilled in the art of antibody engineering to be the amino acid residue at the junction of VL and CL kappa regions in an IgG molecule and is sometimes included in the CL region or, as in Table 3 below, the VL region.

TABLE 3
Sequences of Variable and Constant Regions of IL-1α/IL-1β
DVD-Ig Binding Proteins

| Protein | Sequence | Sequence |
|---|---|---|
| Protein region | Identifier | 12345678901234567890 |
| E26.13-SS-X3 | SEQ ID NO: 46 | EVQLVESGGGVVQPGRSLRL |
| DVD-Ig HEAVY | | SCSASGFIFSRYDMSWVRQA |
| VARIABLE | | PGKGLEWVAYISHGGAGTYY |
| | | PDSVKGRFTISRDNSKNTLF |
| | | LQMDSLRPEDTGVYFCARGG |
| | | VTKGYFDVWGQGTPVTVSSA |
| | | STKGPQVQLVESGGGVVQPG |
| | | RSLRLSCTASGFTFSMFGVH |
| | | WVRQAPGKGLEWVAAVSYDG |
| | | SNKYYAESVKGRFTISRDNS |
| | | KNILFLQMDSLRLEDTAVYY |
| | | CARGRPKVVIPAPLAHWGQG |
| | | TLVTFSS |
| E26.13 VH | SEQ ID NO: 47 | EVQLVESGGGVVQPGRSLRL |
| | | SCSASGFIFSRYDMSWVRQA |
| | | PGKGLEWVAYISHGGAGTYY |
| | | PDSVKGRFTISRDNSKNTLF |
| | | LQMDSLRPEDTGVYFCARGG |
| | | VTKGYFDVWGQGTPVTVSS |
| LINKER | SEQ ID NO: 48 | ASTKGP |
| X3 VH | SEQ ID NO: 49 | QVQLVESGGGVVQPGRSLRL |
| | | SCTASGFTFSMFGVHWVRQA |
| | | PGKGLEWVAAVSYDGSNKYY |
| | | AESVKGRFTISRDNSKNILF |
| | | LQMDSLRLEDTAVYYCARGR |
| | | PKVVIPAPLAHWGQGTLVTF |
| | | SS |
| CH | SEQ ID NO: 50 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| E26.13-SS-X3 | SEQ ID NO: 51 | DIQMTQSPSSLSASVGDRVT |
| DVD-Ig LIGHT | | ITCRASGNIHNYLTWYQQTP |
| VARIABLE | | GKAPKLLIYNAKTLADGVPS |
| | | RFSGSGSGTDYTFTISSLQP |
| | | EDIATYYCQHFWSIPYTFGQ |
| | | GTKLQITRTVAAPDIQMTQS |
| | | PSSVSASVGDRVTITCRASQ |
| | | GISSWLAWYQQKPGKAPKLL |
| | | IYEASNLETGVPSRFSGSGS |
| | | GSDFTLTISSLQPEDFATYY |
| | | CQQTSSFLLSFGGGTKVEHK |
| | | R |
| E26.13 VL | SEQ ID NO: 52 | DIQMTQSPSSLSASVGDRVT |
| | | ITCRASGNIHNYLTWYQQTP |
| | | GKAPKLLIYNAKTLADGVPS |
| | | RFSGSGSGTDYTFTISSLQP |
| | | EDIATYYCQHFWSIPYTFGQ |
| | | GTKLQITR |
| linker | SEQ ID NO: 53 | TVAAP |
| X3 VL | SEQ ID NO: 54 | DIQMTQSPSSVSASVGDRVT |
| | | ITCRASQGISSWLAWYQQKP |
| | | GKAPKLLIYEASNLETGVPS |
| | | RFSGSGSGSDFTLTISSLQP |
| | | EDFATYYCQQTSSFLLSFGG |
| | | GTKVEHKR |
| CL | SEQ ID NO: 55 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |
| E26.13-LL-X3 | SEQ ID NO: 56 | EVQLVESGGGVVQPGRSLRL |
| DVD-Ig HEAVY | | SCSASGFIFSRYDMSWVRQA |
| VARIABLE | | PGKGLEWVAYISHGGAGTYY |
| | | PDSVKGRFTISRDNSKNTLF |
| | | LQMDSLRPEDTGVYFCARGG |
| | | VTKGYFDVWGQGTPVTVSSA |
| | | STKGPSVFPLAPQVQLVESG |
| | | GGVVQPGRSLRLSCTASGFT |
| | | FSMFGVHWVRQAPGKGLEWV |
| | | AAVSYDGSNKYYAESVKGRF |
| | | TISRDNSKNILFLQMDSLRL |
| | | EDTAVYYCARGRPKVVIPAP |
| | | LAHWGQGTLVTFSS |
| E26.13 VH | SEQ ID NO: 57 | EVQLVESGGGVVQPGRSLRL |
| | | SCSASGFIFSRYDMSWVRQA |
| | | PGKGLEWVAYISHGGAGTYY |
| | | PDSVKGRFTISRDNSKNTLF |
| | | LQMDSLRPEDTGVYFCARGG |
| | | VTKGYFDVWGQGTPVTVSS |
| linker | SEQ ID NO: 58 | ASTKGPSVFPLAP |
| X3 VH | SEQ ID NO: 59 | QVQLVESGGGVVQPGRSLRL |
| | | SCTASGFTFSMFGVHWVRQA |
| | | PGKGLEWVAAVSYDGSNKYY |
| | | AESVKGRFTISRDNSKNILF |
| | | LQMDSLRLEDTAVYYCARGR |
| | | PKVVIPAPLAHWGQGTLVTF |
| | | SS |
| CH | SEQ ID NO: 60 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| E26.13-LL-X3 | SEQ ID NO: 61 | DIQMTQSPSSLSASVGDRVT |
| DVD-Ig LIGHT | | ITCRASGNIHNYLTWYQQTP |
| VARIABLE | | GKAPKLLIYNAKTLADGVPS |
| | | RFSGSGSGTDYTFTISSLQP |
| | | EDIATYYCQHFWSIPYTFGQ |
| | | GTKLQITRTVAAPSVFIFPP |
| | | DIQMTQSPSSVSASVGDRVT |
| | | ITCRASQGISSWLAWYQQKP |
| | | GKAPKLLIYEASNLETGVPS |
| | | RFSGSGSGSDFTLTISSLQP |
| | | EDFATYYCQQTSSFLLSFGG |
| | | GTKVEHKR |
| E26.13 VL | SEQ ID NO: 62 | DIQMTQSPSSLSASVGDRVT |
| | | ITCRASGNIHNYLTWYQQTP |
| | | GKAPKLLIYNAKTLADGVPS |
| | | RFSGSGSGTDYTFTISSLQP |
| | | EDIATYYCQHFWSIPYTFGQ |
| | | GTKLQITR |
| linker | SEQ ID NO: 63 | TVAAPSVFIFPP |
| X3 VL | SEQ ID NO: 64 | DIQMTQSPSSVSASVGDRVT |
| | | ITCRASQGISSWLAWYQQKP |
| | | GKAPKLLIYEASNLETGVPS |
| | | RFSGSGSGSDFTLTISSLQP |
| | | EDFATYYCQQTSSFLLSFGG |
| | | GTKVEHKR |
| CL | SEQ ID NO: 65 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |
| X3-SS-E26.13 | SEQ ID NO: 66 | QVQLVESGGGVVQPGRSLRL |
| DVD-Ig HEAVY | | SCTASGFTFSMFGVHWVRQA |
| VARIABLE | | PGKGLEWVAAVSYDGSNKYY |
| | | AESVKGRFTISRDNSKNILF |
| | | LQMDSLRLEDTAVYYCARGR |
| | | PKVVIPAPLAHWGQGTLVTF |
| | | SSASTKGPEVQLVESGGGVV |
| | | QPGRSLRLSCSASGFIFSRY |
| | | DMSWVRQAPGKGLEWVAYIS |
| | | HGGAGTYYPDSVKGRFTISR |
| | | DNSKNTLFLQMDSLRPEDTG |
| | | VYFCARGGVTKGYFDVWGQG |
| | | TPVTVSS |
| X3 VH | SEQ ID NO: 67 | QVQLVESGGGVVQPGRSLRL |
| | | SCTASGFTFSMFGVHWVRQA |
| | | PGKGLEWVAAVSYDGSNKYY |
| | | AESVKGRFTISRDNSKNILF |
| | | LQMDSLRLEDTAVYYCARGR |
| | | PKVVIPAPLAHWGQGTLVTF |
| | | SS |
| linker | SEQ ID NO: 68 | ASTKGP |
| E26.13 VH | SEQ ID NO: 69 | EVQLVESGGGVVQPGRSLRL |
| | | SCSASGFIFSRYDMSWVRQA |
| | | PGKGLEWVAYISHGGAGTYY |
| | | PDSVKGRFTISRDNSKNTLF |
| | | LQMDSLRPEDTGVYFCARGG |
| | | VTKGYFDVWGQGTPVTVSS |
| CH | SEQ ID NO: 70 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| X3-SS-E26.13 | SEQ ID NO: 71 | DIQMTQSPSSVSASVGDRVT |
| DVD-Ig LIGHT | | ITCRASQGISSWLAWYQQKP |
| VARIABLE | | GKAPKLLIYEASNLETGVPS |
| | | RFSGSGSGSDFTLTISSLQP |
| | | EDFATYYCQQTSSFLLSFGG |
| | | GTKVEHKRTVAAPDIQMTQS |
| | | PSSLSASVGDRVTITCRASG |
| | | NIHNYLTWYQQTPGKAPKLL |
| | | IYNAKTLADGVPSRFSGSGS |
| | | GTDYTFTISSLQPEDIATYY |
| | | CQHFWSIPYTFGQGTKLQIT |
| | | R |
| X3 VL | SEQ ID NO: 72 | DIQMTQSPSSVSASVGDRVT |
| | | ITCRASQGISSWLAWYQQKP |
| | | GKAPKLLIYEASNLETGVPS |
| | | RFSGSGSGSDFTLTISSLQP |
| | | EDFATYYCQQTSSFLLSFGG |
| | | GTKVEHKR |
| LINKER | SEQ ID NO: 73 | TVAAP |
| E26.13 VL | SEQ ID NO: 74 | DIQMTQSPSSLSASVGDRVT |
| | | ITCRASGNIHNYLTWYQQTP |
| | | GKAPKLLIYNAKTLADGVPS |
| | | RFSGSGSGTDYTFTISSLQP |
| | | EDIATYYCQHFWSIPYTFGQ |
| | | GTKLQITR |
| CL | SEQ ID NO: 75 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |
| X3-LL-E26.13 | SEQ ID NO: 76 | QVQLVESGGGVVQPGRSLRL |
| DVD-Ig HEAVY | | SCTASGFTFSMFGVHWVRQA |
| VARIABLE | | PGKGLEWVAAVSYDGSNKYY |
| | | AESVKGRFTISRDNSKNILF |
| | | LQMDSLRLEDTAVYYCARGR |
| | | PKVVIPAPLAHWGQGTLVTF |
| | | SSASTKGPSVFPLAPEVQLV |
| | | ESGGGVVQPGRSLRLSCSAS |
| | | GFIFSRYDMSWVRQAPGKGL |
| | | EWVAYISHGGAGTYYPDSVK |
| | | GRFTISRDNSKNTLFLQMDS |
| | | LRPEDTGVYFCARGGVTKGY |
| | | FDVWGQGTPVTVSS |
| X3 VH | SEQ ID NO: 77 | QVQLVESGGGVVQPGRSLRL |
| | | SCTASGFTFSMFGVHWVRQA |
| | | PGKGLEWVAAVSYDGSNKYY |
| | | AESVKGRFTISRDNSKNILF |
| | | LQMDSLRLEDTAVYYCARGR |
| | | PKVVIPAPLAHWGQGTLVTF |
| | | SS |
| LINKER | SEQ ID NO: 78 | ASTKGPSVFPLAP |
| E26.13 VH | SEQ ID NO: 79 | EVQLVESGGGVVQPGRSLRL |
| | | SCSASGFIFSRYDMSWVRQA |
| | | PGKGLEWVAYISHGGAGTYY |
| | | PDSVKGRFTISRDNSKNTLF |
| | | LQMDSLRPEDTGVYFCARGG |
| | | VTKGYFDVWGQGTPVTVSS |
| CH | SEQ ID NO: 80 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| X3-LL-E26.13 | SEQ ID NO: 81 | DIQMTQSPSSVSASVGDRVT |
| DVD-Ig LIGHT | | ITCRASQGISSWLAWYQQKP |
| VARIABLE | | GKAPKLLIYEASNLETGVPS |
| | | RFSGSGSGSDFTLTISSLQP |
| | | EDFATYYCQQTSSFLLSFGG |
| | | GTKVEHKRTVAAPSVFIFPP |
| | | DIQMTQSPSSLSASVGDRVT |
| | | ITCRASGNIHNYLTWYQQTP |
| | | GKAPKLLIYNAKTLADGVPS |
| | | RFSGSGSGTDYTFTISSLQP |
| | | EDIATYYCQHFWSIPYTFGQ |
| | | GTKLQITR |
| X3 VL | SEQ ID NO: 82 | DIQMTQSPSSVSASVGDRVT |
| | | ITCRASQGISSWLAWYQQKP |
| | | GKAPKLLIYEASNLETGVPS |
| | | RFSGSGSGSDFTLTISSLQP |
| | | EDFATYYCQQTSSFLLSFGG |
| | | GTKVEHKR |
| LINKER | SEQ ID NO: 83 | TVAAPSVFIFPP |
| E26.13 VL | SEQ ID NO: 84 | DIQMTQSPSSLSASVGDRVT |
| | | ITCRASGNIHNYLTWYQQTP |
| | | GKAPKLLIYNAKTLADGVPS |
| | | RFSGSGSGTDYTFTISSLQP |
| | | EDIATYYCQHFWSIPYTFGQ |
| | | GTKLQITR |
| CL | SEQ ID NO: 85 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |
| E26.35-SS-X3 JM | SEQ ID NO: 86 | EVQLVESGGGVVQPGRSLRL |
| DVD-Ig HEAVY | | SCSASGFIFSRYDMSWVRQA |
| VARIABLE | | PGKGLEWVAYISHGGAGTYY |
| | | PDSVKGRFTISRDNSKNTLF |
| | | LQMDSLRAEDTAVYYCARGG |
| | | VYKGYFDVWGQGTPVTVSSA |
| | | STKGPQVQLVESGGGVVQPG |
| | | RSLRLSCTASGFTFSMFGVH |
| | | WVRQAPGKGLEWVAAVSYDG |
| | | SNKYYAESVKGRFTISRDNS |
| | | KNILFLQMDSLRLEDTAVYY |
| | | CARGRPKVVIPAPLAHWGQG |
| | | TLVTVSS |
| E26.35 VH | SEQ ID NO: 87 | EVQLVESGGGVVQPGRSLRL |
| | | SCSASGFIFSRYDMSWVRQA |
| | | PGKGLEWVAYISHGGAGTYY |
| | | PDSVKGRFTISRDNSKNTLF |
| | | LQMDSLRAEDTAVYYCARGG |
| | | VYKGYFDVWGQGTPVTVSS |
| LINKER | SEQ ID NO: 88 | ASTKGP |
| X3 JM VH | SEQ ID NO: 89 | QVQLVESGGGVVQPGRSLRL |
| | | SCTASGFTFSMFGVHWVRQA |
| | | PGKGLEWVAAVSYDGSNKYY |
| | | AESVKGRFTISRDNSKNILF |
| | | LQMDSLRLEDTAVYYCARGR |
| | | PKVVIPAPLAHWGQGTLVTV |
| | | SS |
| CH | SEQ ID NO: 90 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| E26.35-SS-X3 JM | SEQ ID NO: 91 | DIQMTQSPSSLSASVGDRVT |
| DVD-Ig LIGHT | | ITCRASGNIHNYLTWYQQTP |
| VARIABLE | | GKAPKLLIYNAKTLADGVPS |
| | | RFSGSGSGTDYTFTISSLQP |
| | | EDIATYYCQHFWSIPYTFGQ |
| | | GTKLQITRTVAAPDIQMTQS |
| | | PSSVSASVGDRVTITCRASQ |
| | | GISSWLAWYQQKPGKAPKLL |
| | | IYEASNLETGVPSRFSGSGS |
| | | GSDFTLTISSLQPEDFATYY |
| | | CQQTSSFLLSFGGGTKVEIK |
| | | R |
| E26.35 VL | SEQ ID NO: 92 | DIQMTQSPSSLSASVGDRVT |
| | | ITCRASGNIHNYLTWYQQTP |
| | | GKAPKLLIYNAKTLADGVPS |
| | | RFSGSGSGTDYTFTISSLQP |
| | | EDIATYYCQHFWSIPYTFGQ |
| | | GTKLQITR |
| LINKER | SEQ ID NO: 93 | TVAAP |
| X3 JM VL | SEQ ID NO: 94 | DIQMTQSPSSVSASVGDRVT |
| | | ITCRASQGISSWLAWYQQKP |
| | | GKAPKLLIYEASNLETGVPS |
| | | RFSGSGSGSDFTLTISSLQP |
| | | EDFATYYCQQTSSFLLSFGG |
| | | GTKVEIKR |
| CL | SEQ ID NO: 95 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |
| E26.13 JM-SS-X3 | SEQ ID NO: 96 | EVQLVESGGGVVQPGRSLRL |
| DVD-Ig HEAVY | | SCSASGFIFSRYDMSWVRQA |
| VARIABLE | | PGKGLEWVAYISHGGAGTYY |
| | | PDSVKGRFTISRDNSKNTLF |
| | | LQMDSLRPEDTGVYFCARGG |
| | | VTKGYFDVWGQGTTVTVSSA |
| | | STKGPQVQLVESGGGVVQPG |
| | | RSLRLSCTASGFTFSMFGVH |
| | | WVRQAPGKGLEWVAAVSYDG |
| | | SNKYYAESVKGRFTISRDNS |
| | | KNILFLQMDSLRLEDTAVYY |
| | | CARGRPKVVIPAPLAHWGQG |
| | | TLVTFSS |
| E26.13 JM VH | SEQ ID NO: 97 | EVQLVESGGGVVQPGRSLRL |
| | | SCSASGFIFSRYDMSWVRQA |
| | | PGKGLEWVAYISHGGAGTYY |
| | | PDSVKGRFTISRDNSKNTLF |
| | | LQMDSLRPEDTGVYFCARGG |
| | | VTKGYFDVWGQGTTVTVSS |
| LINKER | SEQ ID NO: 98 | ASTKGP |
| X3 VH | SEQ ID NO: 99 | QVQLVESGGGVVQPGRSLRL |
| | | SCTASGFTFSMFGVHWVRQA |
| | | PGKGLEWVAAVSYDGSNKYY |
| | | AESVKGRFTISRDNSKNILF |
| | | LQMDSLRLEDTAVYYCARGR |
| | | PKVVIPAPLAHWGQGTLVTF |
| | | SS |
| CH | SEQ ID NO: 100 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| E26.13 JM-SS-X3 | SEQ ID NO: 101 | DIQMTQSPSSLSASVGDRVT |
| DVD-Ig LIGHT | | ITCRASGNIHNYLTWYQQTP |
| VARIABLE | | GKAPKLLIYNAKTLADGVPS |
| | | RFSGSGSGTDYTFTISSLQP |
| | | EDIATYYCQHFWSIPYTFGQ |
| | | GTKLEIKRTVAAPDIQMTQS |
| | | PSSVSASVGDRVTITCRASQ |
| | | GISSWLAWYQQKPGKAPKLL |
| | | IYEASNLETGVPSRFSGSGS |
| | | GSDFTLTISSLQPEDFATYY |
| | | CQQTSSFLLSFGGGTKVEHK |
| | | R |
| E26.13 JM VL | SEQ ID NO: 102 | DIQMTQSPSSLSASVGDRVT |
| | | ITCRASGNIHNYLTWYQQTP |
| | | GKAPKLLIYNAKTLADGVPS |
| | | RFSGSGSGTDYTFTISSLQP |
| | | EDIATYYCQHFWSIPYTFGQ |
| | | GTKLEIKR |
| LINKER | SEQ ID NO: 103 | TVAAP |
| X3 VL | SEQ ID NO: 104 | DIQMTQSPSSVSASVGDRVT |
| | | ITCRASQGISSWLAWYQQKP |
| | | GKAPKLLIYEASNLETGVPS |
| | | RFSGSGSGSDFTLTISSLQP |
| | | EDFATYYCQQTSSFLLSFGG |
| | | GTKVEHKR |
| CL | SEQ ID NO: 105 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |
| E26.35-SS-X3 | SEQ ID NO: 106 | EVQLVESGGGVVQPGRSLRL |
| DVD-Ig HEAVY | | SCSASGFIFSRYDMSWVRQA |
| VARIABLE | | PGKGLEWVAYISHGGAGTYY |
| | | PDSVKGRFTISRDNSKNTLF |
| | | LQMDSLRAEDTAVYYCARGG |
| | | VYKGYFDVWGQGTPVTVSSA |
| | | STKGPQVQLVESGGGVVQPG |
| | | RSLRLSCTASGFTFSMFGVH |
| | | WVRQAPGKGLEWVAAVSYDG |
| | | SNKYYAESVKGRFTISRDNS |
| | | KNILFLQMDSLRLEDTAVYY |
| | | CARGRPKVVIPAPLAHWGQG |
| | | TLVTFSS |
| E26.35 VH | SEQ ID NO: 107 | EVQLVESGGGVVQPGRSLRL |
| | | SCSASGFIFSRYDMSWVRQA |
| | | PGKGLEWVAYISHGGAGTYY |
| | | PDSVKGRFTISRDNSKNTLF |
| | | LQMDSLRAEDTAVYYCARGG |
| | | VYKGYFDVWGQGTPVTVSS |
| LINKER | SEQ ID NO: 108 | ASTKGP |
| X3 VH | SEQ ID NO: 109 | QVQLVESGGGVVQPGRSLRL |
| | | SCTASGFTFSMFGVHWVRQA |
| | | PGKGLEWVAAVSYDGSNKYY |
| | | AESVKGRFTISRDNSKNILF |
| | | LQMDSLRLEDTAVYYCARGR |
| | | PKVVIPAPLAHWGQGTLVTF |
| | | SS |
| CH | SEQ ID NO: 110 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| E26.35-SS-X3 | SEQ ID NO: 111 | DIQMTQSPSSLSASVGDRVT |
| DVD-Ig LIGHT | | ITCRASGNIHNYLTWYQQTP |
| VARIABLE | | GKAPKLLIYNAKTLADGVPS |
| | | RFSGSGSGTDYTFTISSLQP |
| | | EDIATYYCQHFWSIPYTFGQ |
| | | GTKLQITRTVAAPDIQMTQS |
| | | PSSVSASVGDRVTITCRASQ |
| | | GISSWLAWYQQKPGKAPKLL |
| | | IYEASNLETGVPSRFSGSGS |
| | | GSDFTLTISSLQPEDFATYY |
| | | CQQTSSFLLSFGGGTKVEHK |
| | | R |
| E26.35 VL | SEQ ID NO: 112 | DIQMTQSPSSLSASVGDRVT |
| | | ITCRASGNIHNYLTWYQQTP |
| | | GKAPKLLIYNAKTLADGVPS |
| | | RFSGSGSGTDYTFTISSLQP |
| | | EDIATYYCQHFWSIPYTFGQ |
| | | GTKLQITR |
| LINKER | SEQ ID NO: 113 | TVAAP |
| X3 VL | SEQ ID NO: 114 | DIQMTQSPSSVSASVGDRVT |
| | | ITCRASQGISSWLAWYQQKP |
| | | GKAPKLLIYEASNLETGVPS |
| | | RFSGSGSGSDFTLTISSLQP |
| | | EDFATYYCQQTSSFLLSFGG |
| | | GTKVEHKR |
| CL | SEQ ID NO: 115 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |
| E26.13-SS-X3 JM | SEQ ID NO: 116 | EVQLVESGGGVVQPGRSLRL |
| DVD-Ig HEAVY | | SCSASGFIFSRYDMSWVRQA |
| VARIABLE | | PGKGLEWVAYISHGGAGTYY |
| | | PDSVKGRFTISRDNSKNTLF |
| | | LQMDSLRPEDTGVYFCARGG |
| | | VTKGYFDVWGQGTPVTVSSA |
| | | STKGPQVQLVESGGGVVQPG |
| | | RSLRLSCTASGFTFSMFGVH |
| | | WVRQAPGKGLEWVAAVSYDG |
| | | SNKYYAESVKGRFTISRDNS |
| | | KNILFLQMDSLRLEDTAVYY |
| | | CARGRPKVVIPAPLAHWGQG |
| | | TLVTVSS |
| E26.13 VII | SEQ ID NO: 117 | EVQLVESGGGVVQPGRSLRL |
| | | SCSASGFIFSRYDMSWVRQA |
| | | PGKGLEWVAYISHGGAGTYY |
| | | PDSVKGRFTISRDNSKNTLF |
| | | LQMDSLRPEDTGVYFCARGG |
| | | VTKGYFDVWGQGTPVTVSS |
| LINKER | SEQ ID NO: 118 | ASTKGP |
| X3 JM VH | SEQ ID NO: 119 | QVQLVESGGGVVQPGRSLRL |
| | | SCTASGFTFSMFGVHWVRQA |
| | | PGKGLEWVAAVSYDGSNKYY |
| | | AESVKGRFTISRDNSKNILF |
| | | LQMDSLRLEDTAVYYCARGR |
| | | PKVVIPAPLAHWGQGTLVTV |
| | | SS |
| CH | SEQ ID NO: 120 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| E26.13-SS-X3 JM | SEQ ID NO: 121 | DIQMTQSPSSLSASVGDRVT |
| Anti-IL-lalpha/beta | | ITCRASGNIHNYLTWYQQTP |
| DVD-Ig LIGHT | | GKAPKLLIYNAKTLADGVPS |
| VARIABLE | | RFSGSGSGTDYTFTISSLQP |
| | | EDIATYYCQHFWSIPYTFGQ |
| | | GTKLQITRTVAAPDIQMTQS |
| | | PSSVSASVGDRVTITCRASQ |
| | | GISSWLAWYQQKPGKAPKLL |
| | | IYEASNLETGVPSRFSGSGS |
| | | GSDFTLTISSLQPEDFATYY |
| | | CQQTSSFLLSFGGGTKVEIK |
| | | R |
| E26.13 VL | SEQ ID NO: 122 | DIQMTQSPSSLSASVGDRVT |
| | | ITCRASGNIHNYLTWYQQTP |
| | | GKAPKLLIYNAKTLADGVPS |
| | | RFSGSGSGTDYTFTISSLQP |
| | | EDIATYYCQHFWSIPYTFGQ |
| | | GTKLQITR |
| LINKER | SEQ ID NO: 123 | TVAAP |
| X3 JM VL | SEQ ID NO: 124 | DIQMTQSPSSVSASVGDRVT |
| | | ITCRASQGISSWLAWYQQKP |
| | | GKAPKLLIYEASNLETGVPS |
| | | RFSGSGSGSDFTLTISSLQP |
| | | EDFATYYCQQTSSFLLSFGG |
| | | GTKVEIKR |
| CL | SEQ ID NO: 125 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |
| E26.13 JM-LL-X3 | SEQ ID NO: 126 | EVQLVESGGGVVQPGRSLRL |
| DVD-Ig HEAVY | | SCSASGFIFSRYDMSWVRQA |
| VARIABLE | | PGKGLEWVAYISHGGAGTYY |
| | | PDSVKGRFTISRDNSKNTLF |
| | | LQMDSLRPEDTGVYFCARGG |
| | | VTKGYFDVWGQGTTVTVSSA |
| | | STKGPSVFPLAPQVQLVESG |
| | | GGVVQPGRSLRLSCTASGFT |
| | | FSMFGVHWVRQAPGKGLEWV |
| | | AAVSYDGSNKYYAESVKGRF |
| | | TISRDNSKNILFLQMDSLRL |
| | | EDTAVYYCARGRPKVVIPAP |
| | | LAHWGQGTLVTFSS |
| E26.13 JM VH | SEQ ID NO: 127 | EVQLVESGGGVVQPGRSLRL |
| | | SCSASGFIFSRYDMSWVRQA |
| | | PGKGLEWVAYISHGGAGTYY |
| | | PDSVKGRFTISRDNSKNTLF |
| | | LQMDSLRPEDTGVYFCARGG |
| | | VTKGYFDVWGQGTTVTVSS |
| LINKER | SEQ ID NO: 128 | ASTKGPSVFPLAP |
| X3 VH | SEQ ID NO: 129 | QVQLVESGGGVVQPGRSLRL |
| | | SCTASGFTFSMFGVHWVRQA |
| | | PGKGLEWVAAVSYDGSNKYY |
| | | AESVKGRFTISRDNSKNILF |
| | | LQMDSLRLEDTAVYYCARGR |
| | | PKVVIPAPLAHWGQGTLVTF |
| | | SS |
| CH | SEQ ID NO: 130 | ASTKGPSVFPLAPSSKSTSG |
| | | GTAALGCLVKDYFPEPVTVS |
| | | WNSGALTSGVHTFPAVLQSS |
| | | GLYSLSSVVTVPSSSLGTQT |
| | | YICNVNHKPSNTKVDKKVEP |
| | | KSCDKTHTCPPCPAPEAAGG |
| | | PSVFLFPPKPKDTLMISRTP |
| | | EVTCVVVDVSHEDPEVKFNW |
| | | YVDGVEVHNAKTKPREEQYN |
| | | STYRVVSVLTVLHQDWLNGK |
| | | EYKCKVSNKALPAPIEKTIS |
| | | KAKGQPREPQVYTLPPSREE |
| | | MTKNQVSLTCLVKGFYPSDI |
| | | AVEWESNGQPENNYKTTPPV |
| | | LDSDGSFFLYSKLTVDKSRW |
| | | QQGNVFSCSVMHEALHNHYT |
| | | QKSLSLSPGK |
| E26.13 JM-LL-X3 | SEQ ID NO: 131 | DIQMTQSPSSLSASVGDRVT |
| DVD-Ig LIGHT | | ITCRASGNIHNYLTWYQQTP |
| VARIABLE | | GKAPKLLIYNAKTLADGVPS |
| | | RFSGSGSGTDYTFTISSLQP |
| | | EDIATYYCQHFWSIPYTFGQ |
| | | GTKLEIKRTVAAPSVFIFPP |
| | | DIQMTQSPSSVSASVGDRVT |
| | | ITCRASQGISSWLAWYQQKP |
| | | GKAPKLLIYEASNLETGVPS |
| | | RFSGSGSGSDFTLTISSLQP |
| | | EDFATYYCQQTSSFLLSFGG |
| | | GTKVEHKR |
| E26.13 JM VL | SEQ ID NO: 132 | DIQMTQSPSSLSASVGDRVT |
| | | ITCRASGNIHNYLTWYQQTP |
| | | GKAPKLLIYNAKTLADGVPS |
| | | RFSGSGSGTDYTFTISSLQP |
| | | EDIATYYCQHFWSIPYTFGQ |
| | | GTKLEIKR |
| LINKER | SEQ ID NO: 133 | TVAAPSVFIFPP |
| X3 VL | SEQ ID NO: 134 | DIQMTQSPSSVSASVGDRVT |
| | | ITCRASQGISSWLAWYQQKP |
| | | GKAPKLLIYEASNLETGVPS |
| | | RFSGSGSGSDFTLTISSLQP |
| | | EDFATYYCQQTSSFLLSFGG |
| | | GTKVEHKR |
| CL | SEQ ID NO: 135 | TVAAPSVFIFPPSDEQLKSG |
| | | TASVVCLLNNFYPREAKVQW |
| | | KVDNALQSGNSQESVTEQDS |
| | | KDSTYSLSSTLTLSKADYEK |
| | | HKVYACEVTHQGLSSPVTKS |
| | | FNRGEC |

Linker sequences are indicated as underlined residues.

### B. Methods of Treating Osteoarthritis

The compositions, according to the method of the present invention, may be administered using any amount and any route of administration effective for treating osteoarthritis and/or pain associated with the osteoarthritis. Thus, the expression “amount effective for treating osteoarthritis” or “amount effective for treating pain associated with osteoarthritis”, as used herein, refers to a sufficient amount of composition to beneficially prevent or ameliorate the symptoms of osteoarthritis and/or pain associated with the osteoarthritis. The exact dosage is chosen by the individual physician in view of the patient to be treated. Dosage and administration are adjusted to provide sufficient levels of the active agent(s) or to maintain the desired effect. Additional factors which may be taken into account include the severity of the disease state, e.g., intermediate or advanced stage of osteoarthritis; age, weight and gender of the patient; time and frequency of administration; route of administration; drug combinations; reaction sensitivities; area and volume of region of body being treated; and tolerance/response to therapy. Long acting pharmaceutical compositions might be administered hourly, twice hourly, every 3 to four hours, daily, twice daily, every 3 to 4 days, every week, every other week, or once every few weeks or months depending on half-life and clearance rate of the particular composition. The active agents of the invention are preferably formulated in dosage unit form for ease of administration and uniformity of dosage. The expression “dosage unit form” as used herein refers to a physically discrete unit of active agent appropriate for the patient to be treated. It will be understood, however, that the total daily usage of the compositions of the present invention will be decided by the attending physician within the scope of sound medical judgment. For any active agent, the therapeutically effective dose can be estimated initially either in cell culture assays or in animal models, as provided herein, usually mice, but also potentially from rats, rabbits, dogs, or pigs. The animal cell model provided herein is also used to achieve a desirable concentration and total dosing range and route of administration. Such information can then be used to determine useful doses and routes for administration in humans. In some cases clinical data for humans is used to determine an effective dose, however it is understood that doses may be lower or higher based on the specific conditions of a patient or individual being treated.

In various embodiments, the binding protein is administered to a patient using a dosage level of about 0.0001 mg/kg to about 25 mg/kg of body weight. For example the dosage level is calculated per administration or for a period of time, e.g., a day, a week, and a month. In various embodiments, the binding protein is administered at a dose of at least: about 0.0001 mg/kg to about 0.0005 mg/kg; about 0.0005 mg/kg to about 0.001 mg/kg; about 0.001 mg/kg to about 0.005 mg/kg; about 0.005 to about 0.01 mg/kg; about 0.01 mg/kg to about 0.05 mg/kg; about 0.05 mg/kg to 0.1 mg/kg; about 0.1 mg/kg to about 0.5 mg/kg; about 0.05 mg/kg to about 1 mg/kg; about 1 mg/kg to about 2 mg/kg; about 2 mg/kg to about 3 mg/kg; about 3 mg/kg to about 4 mg/kg; about 4 mg/kg to about 5 mg/kg; about 5 mg/kg to about 6 mg/kg; about 6 mg/kg to about 7 mg/kg; about 7 mg/kg to about 8 mg/kg; about 8 mg/kg to about 9 mg/kg; about 9 mg/kg to about 10 mg/kg; about 10 mg/kg to about 11 mg/kg; about 11 mg/kg to about 12 mg/kg; about 12 mg/kg to about 13 mg/kg; about 13 mg/kg to about 14 mg/kg; about 14 mg/kg to about 15 mg/kg; about 15 mg/kg to about 16 mg/kg; about 16 mg/kg to about 17 mg/kg; about 17 mg/kg to about 18 mg/kg; about 18 mg/kg to 19 mg/kg; about 19 mg/kg to about 20 mg/kg; about 20 mg/kg to about 21 mg/kg; about 21 mg/kg to about 22 mg/kg; about 22 mg/kg to about 23 mg/kg; about 23 mg/kg to about 24 mg/kg; and about 24 mg/kg to about 25 mg/kg. Without being limited by any particular theory or mechanism of action, it is here envisioned that osteoarthritis and/or pain associated with the osteoarthritis in an individual can be modulated using different doses of binding protein (e.g., ABT-981 that binds to IL-1α and IL-1β)because of the many factors which must be taken into account in treating these conditions and each individual.

### C. Production of DVD-Ig Binding Proteins

DVD-Ig binding proteins of the present invention may be produced by any of a number of techniques known in the art including, for example, expression from host cells, wherein expression vector(s) encoding the DVD-Ig heavy and DVD-Ig light chains is (are) transfected into a host cell by standard techniques. The various forms of the term “transfection” are intended to encompass a wide variety of techniques commonly used for the introduction of exogenous DNA into a prokaryotic or eukaryotic host cell, e.g., electroporation, calcium-phosphate precipitation, DEAE-dextran transfection and the like. Although it is possible to express the DVD-Ig proteins of the invention in either prokaryotic or eukaryotic host cells, DVD-Ig proteins are expressed in eukaryotic cells, for example, mammalian host cells, because such eukaryotic cells (and in particular mammalian cells) are more likely than prokaryotic cells to assemble and secrete a properly folded and immunologically active DVD-Ig protein.

Exemplary mammalian host cells for expressing the recombinant antibodies of the invention include Chinese Hamster Ovary (CHO cells) (including dhfr-CHO cells, described in Urlaub and Chasin, Proc. Natl. Acad. Sci. USA, 77: 4216-4220 (1980), used with a DHFR selectable marker, e.g., as described in Kaufman and Sharp, J. Mol. Biol., 159: 601-621 (1982)), NSO myeloma cells, COS cells, SP2 and PER.C6 cells. When recombinant expression vectors encoding DVD-Ig proteins are introduced into mammalian host cells, the DVD-Ig proteins are produced by culturing the host cells for a period of time sufficient to allow for expression of the DVD-Ig proteins in the host cells or secretion of the DVD proteins into the culture medium in which the host cells are grown. DVD-Ig proteins can be recovered from the culture medium using standard protein purification methods.

In an exemplary system for recombinant expression of DVD-Ig proteins of the invention, a recombinant expression vector encoding both the DVD-Ig heavy chain and the DVD-Ig light chain is introduced into dhfr-CHO cells by calcium phosphate-mediated transfection. Within the recombinant expression vector, the DVD-Ig heavy and light chain genes are each operatively linked to CMV enhancer/AdMLP promoter regulatory elements to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the DVD-Ig heavy and light chains and intact DVD-Ig protein is recovered from the culture medium. Standard molecular biology techniques are used to prepare the recombinant expression vector, transfect the host cells, select for transformants, culture the host cells and recover the DVD-Ig protein from the culture medium. Still further the invention provides a method of synthesizing a DVD-Ig protein of the invention by culturing a host cell of the invention in a suitable culture medium until a DVD-Ig protein of the invention is synthesized. The method can further comprise isolating the DVD-Ig protein from the culture medium.

An important feature of DVD-Ig is that it can be produced and purified in a similar way as a conventional antibody. The production of DVD-Ig results in a homogeneous, single major product with desired dual-specific activity, without any sequence modification of the constant region or chemical modifications of any kind. Other previously described methods to generate “bi-specific”, “multi-specific”, and “multi-specific multivalent” full length binding proteins do not lead to a single primary product but instead lead to the intracellular or secreted production of a mixture of assembled inactive, mono-specific, multi-specific, multivalent, full length binding proteins, and multivalent full length binding proteins with combination of different binding sites. As an example, based on the design described by Miller and Presta (PCT Publication No. WO 2001/077342, there are 16 possible combinations of heavy and light chains. Consequently only 6.25% of protein is likely to be in the desired active form, and not as a single major product or single primary product compared to the other 15 possible combinations. Separation of the desired, fully active forms of the protein from inactive and partially active forms of the protein using standard chromatography techniques, typically used in large scale manufacturing, is yet to be demonstrated.

### D. Pharmaceutical Compositions

The invention also provides pharmaceutical compositions comprising an antibody (including a DVD-Ig described herein), or antigen-binding portion thereof, of the invention and a pharmaceutically acceptable carrier. The pharmaceutical compositions comprising antibodies of the invention are for use in, but not limited to, diagnosing, detecting, or monitoring a disorder, in preventing, treating, managing, or ameliorating of a disorder or one or more symptoms thereof, and/or in research. In a specific embodiment, a composition comprises one or more antibodies or binding proteins of the invention. In another embodiment, the pharmaceutical composition comprises one or more antibodies of the invention and one or more prophylactic or therapeutic agents other than antibodies of the invention for treating a disorder in which IL-1 (i.e., IL-1α and IL-1β) activity is detrimental, for example osteoarthritis such as hand osteoarthritis and knee osteoarthritis. In an embodiment, the prophylactic or therapeutic agents are known to be useful for or having been or currently being used in the prevention, treatment, management, or amelioration of a disorder or one or more symptoms thereof. In accordance with these embodiments, the composition may further comprise of a carrier, diluent or excipient.

The antibodies and antibody portions of the invention can be incorporated into pharmaceutical compositions suitable for administration to a subject. Typically, the pharmaceutical composition comprises an antibody or antibody portion of the invention and a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible. Examples of pharmaceutically acceptable carriers include one or more of water, saline, phosphate buffered saline, dextrose, glycerol, ethanol and the like, as well as combinations thereof. In many cases, it will be preferable to include isotonic agents, for example, sugars, polyalcohols such as mannitol, sorbitol, or sodium chloride in the composition. Pharmaceutically acceptable carriers may further comprise minor amounts of auxiliary substances such as wetting or emulsifying agents, preservatives or buffers, which enhance the shelf life or effectiveness of the antibody or antibody portion.

In certain embodiments, the binding protein or antibody is formulated in a viable and stable pharmaceutical composition for administration to subjects. See for example, the formulation is prepared as a lyophilized or aqueous formulation. See for example international application numbers WO2014071212 published May 8, 2014; and WO/2013/096835 published Jun. 27, 2013; each of which is here incorporated by references in its entirety. In various embodiments, the formulation lacks physical or chemical instabilities that are undesirable. Examples of problems associated with chemical instability include deamidation, racemization, hydrolysis, oxidation, beta elimination and disulfide exchange. In various embodiments, the formulation comprises a buffer having a physiologically acceptable molarity and pH.

Various delivery systems are known and can be used to administer one or more antibodies of the invention or the combination of one or more antibodies of the invention and a prophylactic agent or therapeutic agent useful for preventing, managing, treating, or ameliorating a disorder or one or more symptoms thereof, e.g., encapsulation in liposomes, microparticles, microcapsules, recombinant cells capable of expressing the antibody or antibody fragment, receptor-mediated endocytosis (see, e.g., Wu and Wu, J. Biol. Chem., 262: 4429-4432 (1987)), construction of a nucleic acid as part of a retroviral or other vector. Methods of administering a prophylactic or therapeutic agent of the invention include, but are not limited to, parenteral administration (e.g., intradermal, intramuscular, intraperitoneal, intravenous and subcutaneous), epidural administration, intratumoral administration, and mucosal administration (e.g., intranasal and oral routes). In addition, pulmonary administration can be employed, e.g., by use of an inhaler or nebulizer, and formulation with an aerosolizing agent. See, e.g., U.S. Pat. Nos. 6,019,968; 5,985,320; 5,985,309; 5,934,272; 5,874,064; 5,855,913 and 5,290,540; and PCT Publication Nos. WO 92/19244, WO 97/32572, WO 97/44013, WO 98/31346, and WO 99/66903, each of which is incorporated herein by reference their entireties. In one embodiment, an antibody or antibody portion of the invention, combination therapy, or a composition of the invention is administered using Alkermes AIR® pulmonary drug delivery technology (Alkermes, Inc., Cambridge, Mass.). In a specific embodiment, prophylactic or therapeutic agents of the invention are administered intramuscularly, intravenously, intratumorally, orally, intranasally, pulmonary, or subcutaneously. The prophylactic or therapeutic agents may be administered by any convenient route, for example by infusion or bolus injection, by absorption through epithelial or mucocutaneous linings (e.g., oral mucosa, rectal, and intestinal mucosa, etc.) and may be administered together with other biologically active agents. Administration can be systemic or local.

In an embodiment, specific binding of antibody-coupled carbon nanotubes (CNTs) to tumor cells in vitro, followed by their highly specific ablation with near-infrared (NIR) light can be used to target tumor cells. For example, biotinylated polar lipids can be used to prepare stable, biocompatible, noncytotoxic CNT dispersions that are then attached to one or two different neutralite avidin-derivatized DVD-Igs directed against one or more tumor antigens (e.g., CD22) (Chakravarty et al., Proc. Natl. Acad. Sci. USA, 105: 8697-8702 (2008)).

In a specific embodiment, it may be desirable to administer the prophylactic or therapeutic agents of the invention locally to the area in need of treatment; this may be achieved by, for example, and not by way of limitation, local infusion, by injection, or by means of an implant, said implant being of a porous or non-porous material, including membranes and matrices, such as sialastic membranes, polymers, fibrous matrices (e.g., Tissuel®), or collagen matrices. In one embodiment, an effective amount of one or more antibodies of the invention antagonists is administered locally to the affected area to a subject to prevent, treat, manage, and/or ameliorate a disorder or a symptom thereof. In another embodiment, an effective amount of one or more antibodies of the invention is administered locally to the affected area in combination with an effective amount of one or more therapies (e.g., one or more prophylactic or therapeutic agents) other than an antibody of the invention of a subject to prevent, treat, manage, and/or ameliorate a disorder or one or more symptoms thereof.

In another embodiment, the prophylactic or therapeutic agent can be delivered in a controlled release or sustained release system. In one embodiment, a pump may be used to achieve controlled or sustained release (see Langer, supra; Sefton, M. V., CRC Crit. Rev. Biomed. Eng., 14: 201-240 (1987); Buchwald et al., Surgery, 88: 507-516 (1980); Saudek et al., N. Engl. J. Med., 321: 574-579 (1989)). In another embodiment, polymeric materials can be used to achieve controlled or sustained release of the therapies of the invention (see, e.g., Goodson, J. M., Chapter 6, In Medical Applications of Controlled Release, Vol. II, Applications and Evaluation, (Langer and Wise, eds.) (CRC Press, Inc., Boca Raton, 1984) pp. 115-138; Langer and Peppas, J. Macromol. Sci. Rev. Macromol. Chem. Phys., C23(1): 61-126 (1983); see also Levy et al., Science, 228:190-192 (1985); During et al., Ann. Neurol., 25:351-356 (1989); Howard et al., J. Neurosurg., 71:105-112 (1989)); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an exemplary embodiment, the polymer used in a sustained release formulation is inert, free of leachable impurities, stable on storage, sterile, and biodegradable. In yet another embodiment, a controlled or sustained release system can be placed in proximity of the prophylactic or therapeutic target, thus requiring only a fraction of the systemic dose (see, e.g., Goodson, in Medical Applications of Controlled Release, supra, vol. 2, pp. 115-138 (1984)).

Controlled release systems are discussed in the review by Langer (Science, 249:1527-1533 (1990)). Any technique known to one of skill in the art can be used to produce sustained release formulations comprising one or more therapeutic agents of the invention. See, e.g., U.S. Pat. No. 4,526,938, PCT Publication No. WO 91/05548, PCT Publication No. WO 96/20698; Ning et al., “Intratumoral radioimmunotherapy of a human colon cancer xenograft using a sustained-release gel,” Radiotherapy Oncol., 39: 179-189 (1996); Song et al., “Antibody Mediated Lung Targeting of Long-Circulating Emulsions,” PDA J. Pharm. Sci. Technol., 50: 372-377 (1996); Cleek et al., “Biodegradable Polymeric Carriers for a bFGF Antibody for Cardiovascular Application,” Proceed. Int'l. Symp. Control. Rel. Bioact. Mater., 24: 853-854 (1997); and Lam et al., “Microencapsulation of Recombinant Humanized Monoclonal Antibody for Local Delivery,” Proceed. Int'l. Symp. Control Rel. Bioact. Mater., 24: 759-760 (1997), each of which is incorporated herein by reference in their entireties.

In a specific embodiment, where the composition of the invention is a nucleic acid encoding a prophylactic or therapeutic agent, the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic®, DuPont), or coating with lipids or cell-surface receptors or transfecting agents, or by administering it in linkage to a homeobox-like peptide which is known to enter the nucleus (see, e.g., Joliot et al., Proc. Natl. Acad. Sci. USA, 88: 1864-1868 (1991)). Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral, e.g., intra-articular, intravenous, intradermal, subcutaneous, oral, intranasal (e.g., inhalation), transdermal (e.g., topical), transmucosal, and rectal administration. In a specific embodiment, the composition is formulated in accordance with routine procedures as a pharmaceutical composition adapted for intravenous, subcutaneous, intramuscular, oral, intranasal, or topical administration to human beings. Typically, compositions for intravenous administration are solutions in sterile isotonic aqueous buffer. Where necessary, the composition may also include a solubilizing agent and a local anesthetic, such as lignocamne, to ease pain at the site of the injection.

If the compositions of the invention are to be administered topically, the compositions can be formulated in the form of an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 19th ed., Mack Pub. Co., Easton, Pa. (1995); and Remington: The Science and Practice of Pharmacy, 22^{nd} ed., Pharmaceutical Press, London, UK (2005). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as FREON®) or in a squeeze bottle. Moisturizers or humectants can also be added to pharmaceutical compositions and dosage forms if desired. Examples of such additional ingredients are well known in the art.

If the method of the invention comprises intranasal administration of a composition, the composition can be formulated in an aerosol form, spray, mist or in the form of drops. In particular, prophylactic or therapeutic agents for use according to the present invention can be conveniently delivered in the form of an aerosol spray presentation from pressurized packs or a nebulizer, with the use of a suitable propellant (e.g., dichlorodifluoromethane, trichlorofluoromethane, dichlorotetrafluoroethane, carbon dioxide or other suitable gas). In the case of a pressurized aerosol the dosage unit may be determined by providing a valve to deliver a metered amount. Capsules and cartridges (composed of, e.g., gelatin) for use in an inhaler or insufflator may be formulated containing a powder mix of the compound and a suitable powder base such as lactose or starch.

If the method of the invention comprises oral administration, compositions can be formulated orally in the form of tablets, capsules, cachets, gelcaps, solutions, suspensions, and the like. Tablets or capsules can be prepared by conventional means with pharmaceutically acceptable excipients such as binding agents (e.g., pregelatinised maize starch, polyvinylpyrrolidone, or hydroxypropyl methylcellulose); fillers (e.g., lactose, microcrystalline cellulose, or calcium hydrogen phosphate); lubricants (e.g., magnesium stearate, talc, or silica); disintegrants (e.g., potato starch or sodium starch glycolate); or wetting agents (e.g., sodium lauryl sulphate). The tablets may be coated by methods well-known in the art. Liquid preparations for oral administration may take the form of, but not limited to, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives, or hydrogenated edible fats); emulsifying agents (e.g., lecithin or acacia); non-aqueous vehicles (e.g., almond oil, oily esters, ethyl alcohol, or fractionated vegetable oils); and preservatives (e.g., methyl or propyl-p-hydroxybenzoates or sorbic acid). The preparations may also contain buffer salts, flavoring, coloring, and sweetening agents as appropriate. Preparations for oral administration may be suitably formulated for slow release, controlled release, or sustained release of a prophylactic or therapeutic agent(s).

The method of the invention may comprise pulmonary administration, e.g., by use of an inhaler or nebulizer, of a composition formulated with an aerosolizing agent. See, e.g., U.S. Pat. Nos. 6,019,968; 5,985,320; 5,985,309; 5,934,272; 5,874,064; 5,855,913; and 5,290,540; and PCT Publication Nos. WO 92/19244, WO 97/32572, WO 97/44013, WO 98/31346, and WO 99/66903, each of which is incorporated herein by reference their entireties. In a specific embodiment, an antibody of the invention, combination therapy, and/or composition of the invention is administered using Alkermes AIR® pulmonary drug delivery technology (Alkermes, Inc., Cambridge, Mass.).

The method of the invention may comprise administration of a composition formulated for parenteral administration by injection (e.g., by bolus injection or continuous infusion). Alternatively, the composition including the binding protein is formulated for local administration. Formulations for injection may be presented in unit dosage form (e.g., in ampoules or in multi-dose containers) with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle (e.g., sterile pyrogen-free water) before use.

In various embodiments, administering involves directly contacting an arthritic area or pain-affected area, e.g., a knee, foot, toe, wrist, finger, ankle shoulder, or a disc. In various embodiments, administering the binding protein has a systemic effect. In various embodiments, administering involves contacting an adjacent tissue or area with the composition comprising the binding protein and allowing migration or diffusion of the binding protein to the arthritic area or pain-affected area of the individual or patient. For example the binding protein is administered to the epidural space of the back or a vessel/artery that contacts the arthritic area or the pain-affected area.

The methods of the invention may additionally comprise of administration of compositions formulated as depot preparations. Such long acting formulations may be administered by implantation (e.g., subcutaneously or intramuscularly) or by intramuscular injection. Thus, for example, the compositions may be formulated with suitable polymeric or hydrophobic materials (e.g., as an emulsion in an acceptable oil) or ion exchange resins, or as sparingly soluble derivatives (e.g., as a sparingly soluble salt).

The methods of the invention encompass administration of compositions formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions are supplied either separately or mixed together in unit dosage form, for example, as a dry lyophilized powder or water free concentrate in a hermetically sealed container such as an ampoule or sachet indicating the quantity of active agent. Where the mode of administration is infusion, composition can be dispensed with an infusion bottle containing sterile pharmaceutical grade water or saline. Where the mode of administration is by injection, an ampoule of sterile water for injection or saline can be provided so that the ingredients may be mixed prior to administration.

In particular, the invention also provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted (e.g., with water or saline) to the appropriate concentration for administration to a subject. Preferably, one or more of the prophylactic or therapeutic agents or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents or pharmaceutical compositions of the invention should be stored at between 2° C. and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

In various embodiments, the binding protein is administered at a dose that is identified by a physician based on specific conditions of the individual being treated. For example, the size of the area to be treated, extent of osteoarthritis, and level of pain may affect the dose that is administered to the individual/patient.

In various embodiments, the binding protein is administered at a dose about 0.005 (milligrams per kilogram) mg/kg to about 0.01 mg/kg, about 0.01 mg/kg to about 0.05 mg/kg, about 0.05 mg/kg to about 0.1 mg/kg, about 0.1 mg/kg to about 1 mg/kg, about 1 mg/kg to about 2 mg/kg, about 2 mg/kg to about 3 mg/kg, about 3 mg/kg to about 4 mg/kg, about 4 mg/kg to about 5 mg/kg, about 5 mg/kg to about 6 mg/kg, about 6 mg/kg to about 7 mg/kg, about 7 mg/kg to about 8 mg/kg, about 8 mg/kg to about 9 mg/kg, or about 9 mg/kg to about 10 mg/kg of weight of the binding protein to weight of the individual.

The antibodies and antibody portions of the invention can be incorporated into a pharmaceutical composition suitable for parenteral administration. Preferably, the antibody or antibody-portions will be prepared as an injectable solution containing 0.1-250 mg/ml antibody. The injectable solution can be composed of either a liquid or lyophilized dosage form in a flint or amber vial, ampoule or pre-filled syringe. The buffer can be L-histidine (1-50 mM), optimally 5-10 mM, at pH 5.0 to 7.0 (optimally pH 6.0). Other suitable buffers include but are not limited to, sodium succinate, sodium citrate, sodium phosphate or potassium phosphate. Sodium chloride can be used to modify the toxicity of the solution at a concentration of 0-300 mM (optimally 150 mM for a liquid dosage form). Cryoprotectants can be included for a lyophilized dosage form, principally 0-10% sucrose (optimally 0.5-1.0%). Other suitable cryoprotectants include trehalose and lactose. Bulking agents can be included for a lyophilized dosage form, principally 1-10% mannitol (optimally 2-4%). Stabilizers can be used in both liquid and lyophilized dosage forms, principally 1-50 mM L-Methionine (optimally 5-10 mM). Other suitable bulking agents include glycine, arginine, can be included as 0-0.05% polysorbate-80 (optimally 0.005-0.01%). Additional surfactants include but are not limited to polysorbate 20 and BRIJ surfactants. The pharmaceutical composition comprising an antibody or antibody portion of the invention prepared as an injectable solution for parenteral administration, can further comprise an agent useful as an adjuvant, such as those used to increase the absorption, or dispersion of a therapeutic protein (e.g., antibody). A particularly useful adjuvant is hyaluronidase (such as Hylenex® recombinant human hyaluronidase). Addition of hyaluronidase in the injectable solution improves human bioavailability following parenteral administration, particularly subcutaneous administration. It also allows for greater injection site volumes (i.e., greater than 1 ml) with less pain and discomfort, and minimum incidence of injection site reactions (see, PCT Publication No. WO 2004/078140 and US Publication No. 2006/104968).

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for passive immunization of humans with other antibodies. The preferred mode of administration is parenteral (e.g., intravenous, subcutaneous, intraperitoneal, intramuscular). In an exemplary embodiment, the antibody is administered by intravenous infusion or injection. In another preferred embodiment, the antibody is administered by intramuscular or subcutaneous injection.

Therapeutic compositions typically must be sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e., antibody or antibody portion) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile, lyophilized powders for the preparation of sterile injectable solutions, the preferred methods of preparation are vacuum drying and spray-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including, in the composition, an agent that delays absorption, for example, monostearate salts and gelatin.

The binding proteins of the present invention can be administered by a variety of methods known in the art, although for many therapeutic applications, the preferred route/mode of administration is subcutaneous injection, intravenous injection or infusion. As will be appreciated by the skilled artisan, the route and/or mode of administration will vary depending upon the desired results. In certain embodiments, the active compound may be prepared with a carrier that will protect the compound against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known to those skilled in the art. See, e.g., Sustained and Controlled Release Drug Delivery Systems, (J. R. Robinson, ed.) (Marcel Dekker, Inc., New York, 1978).

In certain embodiments, an antibody or antibody portion of the invention may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Supplementary active compounds can also be incorporated into the compositions. In certain embodiments, an antibody or antibody portion of the invention is co-formulated with and/or co-administered with one or more additional therapeutic agents that are useful for treating disorders in which IL-1α and/or IL-1β activity is detrimental. For example, an anti-human IL-1α/IL-1β antibody or antibody portion of the invention may be co-formulated and/or co-administered with one or more additional antibodies that bind other targets (e.g., antibodies that bind other cytokines or that bind cell surface molecules). Furthermore, one or more antibodies of the invention may be used in combination with two or more of the foregoing therapeutic agents. Such combination therapies may advantageously utilize lower dosages of the administered therapeutic agents, thus avoiding possible toxicities or complications associated with the various monotherapies.

It should further be understood that the combinations which are to be included within this invention are those combinations useful for their intended purpose. The agents set forth below are illustrative for purposes and not intended to be limited. The combinations, which are part of this invention, can be the antibodies of the present invention and at least one additional agent selected from the lists below. The combination can also include more than one additional agent, e.g., two or three additional agents if the combination is such that the formed composition can perform its intended function.

Preferred combinations are non-steroidal anti-inflammatory drug(s) also referred to as NSAIDS which include drugs like ibuprofen. Other preferred combinations are corticosteroids including prednisolone; the well-known side-effects of steroid use can be reduced or even eliminated by tapering the steroid dose required when treating patients in combination with the anti-IL-1α and anti-IL-1β antibodies of this invention. Non-limiting examples of therapeutic agents for rheumatoid arthritis with which an antibody or antibody portion of the invention can be combined include, but are not limited to, the following: cytokine suppressive anti-inflammatory drug(s) (CSAIDs); antibodies to or antagonists of other human cytokines or growth factors, for example, TNF, LT, IL-1, IL-2, IL-3, IL-4, IL-5, IL-6, IL-7, IL-8, IL-15, IL-16, IL-18, IL-21, interferons, EMAP-II, GM-CSF, FGF, and PDGF. Antibodies of the invention, or antigen binding portions thereof, can be combined with antibodies to cell surface molecules such as CD2, CD3, CD4, CD8, CD25, CD28, CD30, CD40, CD45, CD69, CD80 (B7.1), CD86 (B7.2), CD90, CTLA or their ligands including CD154 (gp39 or CD40L).

Preferred combinations of therapeutic agents may interfere at different points in the autoimmune and subsequent inflammatory cascade; preferred examples include TNF antagonists like chimeric, humanized or human TNF antibodies, D2E7, (PCT Publication No. WO 97/29131), CA2 (Remicade™) CDP 571, and soluble p55 or p75 TNF receptors, derivatives, thereof, (p75TNFR1gG (Enbrel™) or p55TNFR1gG (Lenercept), and also TNFαconverting enzyme (TACE) inhibitors; similarly IL-1 inhibitors (Interleukin-1-converting enzyme inhibitors, IL-1RA etc.) may be effective for the same reason. Other preferred combinations include Interleukin 11. Yet another preferred combination are other key players of the autoimmune response which may act parallel to, dependent on or in concert with IL-1β function. Yet another preferred combination includes non-depleting anti-CD4 inhibitors. Yet other preferred combinations include antagonists of the co-stimulatory pathway CD80 (B7.1) or CD86 (B7.2) including antibodies, soluble receptors or antagonistic ligands.

The antibodies of the invention, or antigen binding portions thereof, may also be combined with agents, such as methotrexate, 6-MP, azathioprine sulphasalazine, mesalazine, olsalazine chloroquinine/hydroxychloroquine, pencillamine, aurothiomalate (intramuscular and oral), azathioprine, colchicine, corticosteroids (oral, inhaled and local injection), beta-2 adrenoreceptor agonists (salbutamol, terbutaline, salmeteral), xanthines (theophylline, aminophylline), cromoglycate, nedocromil, ketotifen, ipratropium and oxitropium, cyclosporin, FK506, rapamycin, mycophenolate mofetil, leflunomide, NSAIDs, for example, ibuprofen, corticosteroids such as prednisolone, phosphodiesterase inhibitors, adensosine agonists, antithrombotic agents, complement inhibitors, adrenergic agents, agents which interfere with signaling by proinflammatory cytokines such as TNF-α or IL-1 (e.g., IRAK, NIK, IKK, p38, or MAP kinase inhibitors), IL-1β converting enzyme inhibitors, TNF-αconverting enzyme (TACE) inhibitors, T-cell signaling inhibitors such as kinase inhibitors, metalloproteinase inhibitors, sulfasalazine, azathioprine, 6-mercaptopurines, angiotensin converting enzyme inhibitors, soluble cytokine receptors and derivatives thereof (e.g., soluble p55 or p75 TNF receptors and the derivatives p75TNFRIgG (Enbrel™ and p55TNFRIgG (Lenercept)), sIL-1RI, sIL-1RII, sIL-6R), antiinflammatory cytokines (e.g., IL-4, IL-1β, IL-11, IL-13 and TGFβ), celecoxib, folic acid, hydroxychloroquine sulfate, rofecoxib, etanercept, infliximab, naproxen, valdecoxib, sulfasalazine, methylprednisolone, meloxicam, methylprednisolone acetate, gold sodium thiomalate, aspirin, triamcinolone acetonide, propoxyphene napsylate/apap, folate, nabumetone, diclofenac, piroxicam, etodolac, diclofenac sodium, oxaprozin, oxycodone hcl, hydrocodone bitartrate/apap, diclofenac sodium/misoprostol, fentanyl, anakinra, human recombinant, tramadol HCL, salsalate, sulindac, cyanocobalamin/fa/pyridoxine, acetaminophen, alendronate sodium, prednisolone, morphine sulfate, lidocaine hydrochloride, indomethacin, glucosamine sulf/chondroitin, amitriptyline hcl, sulfadiazine, oxycodone HCL/acetaminophen, olopatadine hcl, misoprostol, naproxen sodium, omeprazole, cyclophosphamide, rituximab, IL-1 TRAP, MRA, CTLA4-IG, IL-18 BP, anti-IL-18, anti-IL15, BIRB-796, SC10-469, VX-702, AMG-548, VX-740, Roflumilast, IC-485, CDC-801, and Mesopram.

The pharmaceutical compositions of the invention may include a “therapeutically effective amount” or a “prophylactically effective amount” of an antibody or antibody portion of the invention. A “therapeutically effective amount” refers to an amount effective, at dosages and for periods of time necessary, to achieve the desired therapeutic result. A therapeutically effective amount of the antibody or antibody portion may be determined by a person skilled in the art and may vary according to factors such as the disease state, age, sex, and weight of the individual, and the ability of the antibody or antibody portion to elicit a desired response in the individual. A therapeutically effective amount is also one in which any toxic or detrimental effects of the antibody, or antibody portion, are outweighed by the therapeutically beneficial effects. A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Dosage regimens may be adjusted to provide the optimum desired response (e.g., a therapeutic or prophylactic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the mammalian subjects to be treated; each unit containing a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic or prophylactic effect to be achieved, and (b) the limitations inherent in the art of compounding such an active compound for the treatment of sensitivity in individuals.

It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

It will be readily apparent to those skilled in the art that other suitable modifications and adaptations of the methods of the invention described herein are obvious and may be made using suitable equivalents without departing from the scope of the invention or the embodiments disclosed herein.

Having now described the present invention in detail, the same will be more clearly understood by reference to the following examples, which are included for purposes of illustration only and are not intended to be limiting of the invention.

### EXEMPLIFICATIONS

### Example 1

### Safety, Tolerability and Pharmacokinetics of ABT-981, an IL-1α and IL-1β Dual Target Biologic Drug in Development for Osteoarthritis, Following Single Dose Administration in Healthy Subjects; Phase 1 Study Trial 1

A randomized, double-blind, placebo-controlled Phase 1 study was conducted to assess ABT-981 following a single intravenous (IV) infusion (0.3, 1, 3 or 10 mg/kg) or a single subcutaneous (SC) injection (0.3, 1 or 3 mg/kg). Fifty-six male and female healthy volunteers, 18 to 55 years old, were enrolled in this study. In addition, subjects had to have a condition of general good health based on medical history, physical exam, vital signs, laboratory profile, chest x-ray, and a 12-lead ECG. Furthermore, subject's body mass index (BMI) was 18 to 29.9 kg/m², inclusive, at screening.

Subjects were excluded if they had a previous exposure to anti-IL-1 treatment. Furthermore, subjects were excluded if he or she had had a positive screen for drugs of abuse, alcohol or nicotine. Still further, subjects were excluded if he or she had used any over-the-counter and/or prescription medication, vitamins, and/or herbal supplements two weeks prior to study drug administration. Female subjects who were considering becoming pregnant or male subjects who were considering fathering a child during the study for approximately three months after the last dose of study drug were also excluded.

In each dose cohort, six subjects received active ABT-981 drug and two received placebo. Safety assessment and PK/ADA samples were collected for 84 days following dosing. Subjects were confined through Study Day 8. Subjects returned for safety and pharmacokinetic evaluations on Study Days 11, 15, 22, 29, 36, 43, 57, 71, and 85. Intensive pharmacokinetic monitoring, ADA monitoring, and safety monitoring were performed. The Pharmacokinetic and ADA monitoring involved two parts. Part 1 involved PK analysis prior to the dose on study day 1 (0-hour) and at 2, 4, 6, 10, 14 hours after the start of infusion as well as on study days 2, 3, 4, 5, 6, 7, 8, 11, 15, 22, 29, 36, 43, 57, 71, and 85. ADA analysis was performed on study days 15, 22, 29, 36, 43, 57, 71 and 85 after the start of the infusion. Part 2 of the pharmacokinetic and ADS monitoring involved PK analysis prior to the dose on study day 1 (0-hour) and at 8 hours after the SC injection as well as on study days, 2, 3, 4, 5, 6, 7, 8, 11, 15, 22, 29, 36, 43, 57, 71, and 85. ADA monitoring on study days 15, 22, 29, 36, 43, 57, 71 and 85 was performed after the SC injection. Adverse events were coded using Medical Dictionary for Regulatory Activities (MedDRA) version 16.0. In addition, safety analysis of subjects was performed by monitoring adverse events, and performing vital signs, physical examination, ECG, laboratory tests assessments.

### Pharmacokinetic Variables

Data show that the maximum observed concentration (Cmax) and area under the curve (AUC∞) for subjects administered ABT-981 increased in an approximately dose-proportional manner after single dose administration from 0.3 mg/kg to 10 mg/kg IV and 0.3 mg/kg to 3 mg/kg SC (see Table 4 and Table 5).

TABLE 4
Pharmacokinetic parameters for
ABT-981 following a single intravenous close of ABT-981

| | Intravenous Doses [Mean (% CV)] | | | |
|---|---|---|---|---|
| | Group 1 | Group 2 | Group 3 | Group 4 |
| Parameter | 0.3 mg/kg | 1 mg/kg | 3 mg/kg | 10 mg/kg |
| N | 6 | 6 | 6 | 6 |
| Tₘₐₓ (hr) | 5.7 [2-10] | 3.3 [2-4] | 4.0 [2-6] | 4.4 [4-6] |
| [range] | | | | |
| Cₘₐₓ (μg/mL) | 8.8 (11) | 23.5 (15) | 82.2 (8) | 275 (8) |
| AUC_{inf} | 1860 (27) | 6470 (25) | 18100 (19) | 56600 (18) |
| (μg · hr/mL) | | | | |
| t_{1/2} (hr) | 282 (28) | 322 (21) | 281 (26) | 291 (15) |
| harmonic | | | | |
| mean | | | | |
| Subjects with | 2 (33.3) | 0 (0.0) | 2 (33.3) | 0 (0.0) |
| Measurable | | | | |
| ADA [n (%)]^{a} | | | | |

^{a}The IV placebo group had 1 subject (12.5%) with measurable ADA.

TABLE 5
Pharmacokinetic parameters of ABT-981 following a single
subcutaneous close of ABT-981

| | Subcutaneous Doses [Mean (% CV)] | | |
|---|---|---|---|
| | Group 5 | Group 6 | Group 7 |
| Parameter | 0.3 mg/kg | 1 mg/kg | 3 mg/kg |
| N | 6 | 5^{b} | 6 |
| Tₘₐₓ (hr) [range] | 124 [72-240] | 120 [72-168] | 116 [48-144] |
| Cₘₐₓ (μg/mL) | 1.4 (29) | 5.9 (48) | 18.1 (15) |
| AUC_{inf} (μg · hr/mL) | 755 (25) | 3370 (46) | 9600 (14) |
| t_{1/2} (hr) | 281 (10) | 347 (20) | 284 (10) |
| harmonic mean | | | |
| Subjects with | 1 (16.7) | 1 (20) | 1 (16.7) |
| Measurable ADA | | | |
| [n (%)]^{c} | | | |

^{a}One subject in Group 6 had no measurable ABT-981 concentrations following dosing and was excluded from the analyses.
^{b}The SC placebo group had 1 subject (16.7%) with measurable ADA.

### Safety Summary

Overall rates of occurrence and severity of adverse effects (AEs) were similar between ABT-981 treated and placebo groups. All AEs occurred in the ABT-981 treated subjects were mild or moderate in severity with the exception of 1 severe event of transaminases increased in a subject in the 1 mg/kg IV group—it was considered as having no reasonable possibility of being related to the study drug.

Four subjects had an AE of mild neutropenia; however three of the four had lower baseline neutrophil counts (<2000 cells/mm3) There was no clear association of neutropenia with other AEs, including infections. There were no AEs related to chemistry or urinary values, vital signs, or cardiac parameters. There was 1 serious AE (splenic infarction) experienced by a subject who received placebo IV. No deaths or SAEs were reported with ABT-981 IV or SC and no subjects were discontinued from the study following dosing with ABT-981 IV or SC due to an AE. Favorable half-life profiles (11-14 days) for the ABT-981 protein were observed. Furthermore, data show low occurrence of anti-drug antibodies in patient serum samples.

Following intravenous dosing, sixteen subjects (16/24; 66.7%) who received ABT-981 reported 1 or more adverse events compared to the four subjects (4/8; 50.0%) who received placebo. The majority (12/16) of the adverse events observed in subjects were deemed unrelated to administration of the study drug. The adverse events reported were either mild or moderate in severity with the exception of one severe event of transaminases increased in a subject in the 1 mg/kg intravenous group.

Ten subjects (10/18; 55.6%) who received ABT 981 subcutaneously reported one or more adverse effects compared to the four control subjects (4/6; 66.7%) who received the placebo subcutaneously. Most importantly, the majority of the adverse effects observed were determined to be unrelated to administration of the study drug.

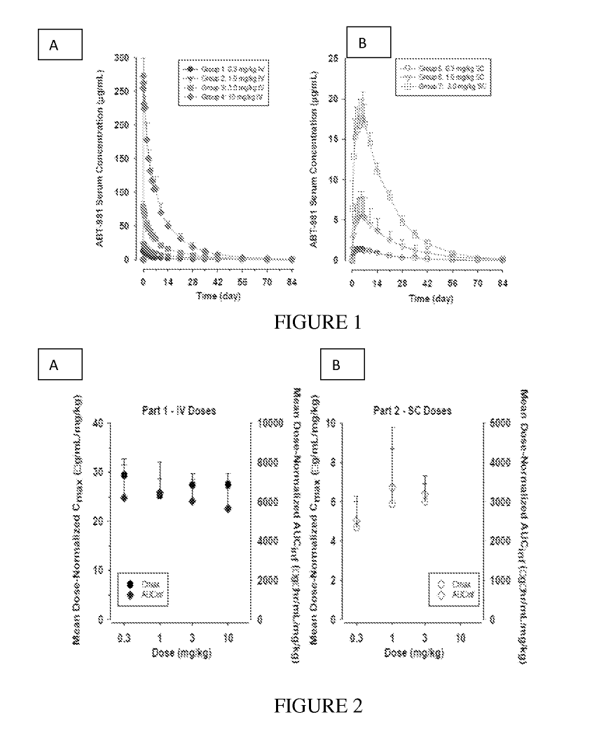

In summary, ABT-981 was analyzed herein in the first-in-human (FIH) single ascending dose two part study including intravenous infusion (0.3, 1, 3, 10 mg/kg), and subcutaneous injection (0.3, 1, and 3.0 mg/kg) in healthy subjects. See FIG. 1 panels A and B, and FIG. 2 panels A and B. The highest average maximum observed serum concentration (Cmax; 275 μg/mL) and area under the serum concentration-time curve from time zero to infinity (AUCinf; 56,600 μg·h/mL) were observed following the 10 mg/kg infusion. The Cmax and AUC values appeared approximately dose proportional from 0.3 mg/kg to 10 mg/kg intravenous dosing and 0.3 to 3 mg/kg subcutaneous dosing. Data show that the terminal phase elimination half-life (t½) ranged from 11 to 14 days and was independent of administration route. Mean time to maximum observed serum concentration (Tmax) after subcutaneous dosing was five days. Measurable ADA titers were observed in 9 subjects (9/55, 16.4%) including two placebo subjects. No apparent ADA effect on ABT-981 pharmacokinetics was observed. No difference was observed in ADA incidence following intravenous versus subcutaneous administration, and there was no clear correlation between ADA incidence and dose. Exposures in healthy subjects increased in an approximately dose-proportional manner after single dose IV and SC administration of ABT-981. ABT-981 was well tolerated in healthy subjects administered single ABT-981 doses either via IV infusion or SC. This human study supports further investigation of this DVD-Ig™ protein following multiple doses in an OA population.

### Example 2

### Treatment of Patients Having Knee Osteoarthritis Using IL-1α/β Binding Protein in Phase 1 Trial 2 Study

This study was a randomized, double-blind, multiple ascending dose (MAD), placebo-controlled trial designed to assess the safety, tolerability, PK and pharmacodynamics (PD) of multiple subcutaneous (SC) injections of ABT-981 in knee OA patients. Subjects were males or females whose ages were between 40 and 70 years. Subjects had a diagnosis of chronic, symptomatic, mild to moderate radiographic knee OA and were otherwise in general good health based upon the results of a medical history, physical examination, vital signs, laboratory profile, chest x-ray and 12-lead electrocardiogram (ECG). Females were postmenopausal for at least 2 years, surgically sterile, sexually inactive or practiced birth control, and were not pregnant or breastfeeding. Males were surgically sterile, sexually inactive or practiced birth control. Knee OA patients were divided into three groups. Each group of patients received four doses of either ABT-981 DVD-Ig or matching placebo (7:2) every two weeks (E2W or EOW). The three groups were SC administered distinct dose levels every other week: 0.3 mg/kg (lower dose; Low Dose EOW), 1 mg/kg (middle dose; MID Dose EOW), or 3 mg/kg (higher dose; High Dose EOW). A fourth group was administered 3 mg/kg ABT-981 or placebo SC; subjects were administered three doses once E4W. The dose amount identifiers low, middle and higher are relative terms used herein, and are not meant to limit the amount/dose that may be administered to a patient under certain circumstances identified by a physician. Serum samples were collected, e.g., days 1, 5, 15, 19, 29, 33, 43, 47, and 57. Additional urine and serum samples for a subset of biomarkers were also collected throughout the study, e.g., days 3, 10, 14, 28, 42, and 45. Serum concentrations of ABT 981 were determined using a validated chimeric electrochemiluminescence (ECL) immunoassay in bridging format. Values for the pharmacokinetic parameters of ABT-981 were estimated using non compartmental methods: maximum observed serum concentration (Cmax), the time to Cmax (peak time, Tmax), the observed serum concentration prior to dose (Ctrough) and the area under the concentration time curve (AUC) from time 0 to the time of the last measurable concentration (AUCt) and AUC from the time zero to time of next dose interval (AUCtau) were estimated after the first and the fourth doses for Dosing Groups 1 through 3 and after the first and third doses for Dosing Group 4. The terminal phase elimination rate constant (β), the terminal elimination half-life (t½), the AUC from time 0 to infinite time (AUC∞) and apparent oral plasma clearance (CL/F) were determined using non-compartmental methods after the final dose in all groups.

No apparent ADA effect on ABT-981 PK was observed in either phase 1 trial. The safety profile and incidence of adverse events were similar between subjects receiving ABT-981 or placebo. Pharmacokinetic data for SC administration in this current study are shown in Table 6. The total variability in Cmax and AUCtau, for ABT-981, expressed as percent CV, in all four dose groups, is shown in Table 7. Safety and tolerability were assessed by adverse event assessments, vital signs monitoring, physical examinations, ECGs and laboratory value assessments. ADA titers were determined

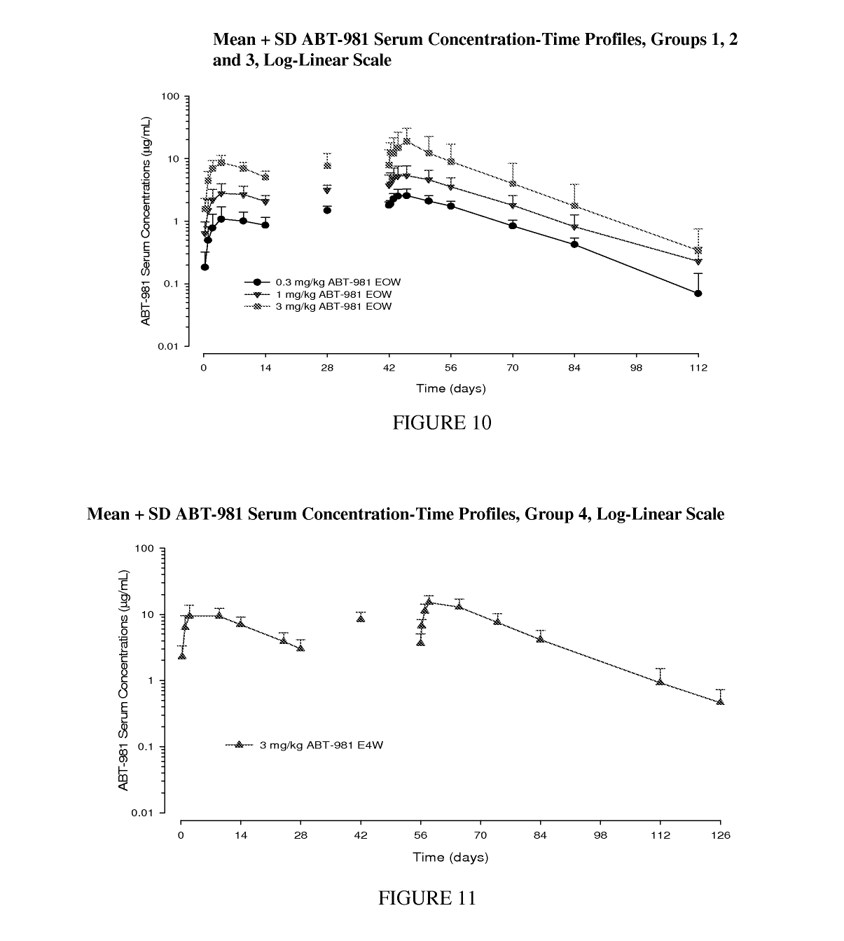

Data show that ABT-981 reached a Tmax from 3 days to 7 days after dosing with mean terminal half-life of 10 to 13 days. After 4 EOW doses the mean Cmax and AUCτ were 2.59-22.6 μg/mL and 30.7-248 μg·day/mL at 0.3-3.0 mg/kg (FIG. 10 and FIG. 11). Data show that exposures increased approximately linearly between 0.3 and 3 mg/kg and accumulation was approximately 2-fold. ABT-981 exhibited behavior similar to a conventional antibody with linear pharmacokinetics. The ABT-981 PK profile shown in data supports EOW or E4W dosing of ABT-981. Without being limited by any particular theory or mechanism of action, it is here envisioned that ABT-981 is efficacious, therapeutic, safe, and produces beneficial biochemical and/or human therapeutic effects (i.e., improved metrics and scores) in the patients having knee osteoarthritis.

TABLE 6
Pharmacokinetic Results:
The pharmacokinetic parameters (mean ± SD) of ABT-981 for all four dose
groups following the first and last doses are summarized in in the following table.

| | | Group 1 | Group 2 | Group 3 | Group 4 |
|---|---|---|---|---|---|
| Pharmacokinetic | | 0.3 mg/kg | 1.0 mg/kg | 3.0 mg/kg | 3.0 mg/kg |
| Parameter | Units | EOW | EOW | EOW | E4W |
| | | First Dose | | | |
| N | | 7 | 7 | 7 | 7 |
| Cₘₐₓ | μg/mL | 1.14 ± 0.59 | 2.84 ± 1.17 | 8.72 ± 2.59 | 10.4 ± 3.89 |
| Tₘₐₓ | day | 6.86 ± 2.67 | 5.43 ± 2.44 | 5.43 ± 2.44 | 6.00 ± 3.74 |
| AUCₜₐᵤ^{a} | μg · day/mL | 12.6 ± 5.95 | 33.0 ± 12.7 | 92.9 ± 25.3 | 186 ± 61.2 |
| Cₘₐₓ/Dose | (μg/mL)/(mg/kg) | 3.78 ± 1.97 | 2.84 ± 1.17 | 2.91 ± 0.86 | 3.46 ± 1.30 |
| AUCₜₐᵤ/Dose | (μg · day/mL)/(mg/kg) | 42.1 ± 19.8 | 33.0 ± 12.7 | 31.0 ± 8.42 | 61.9 ± 20.4 |
| | | Last Doseb | | | |
| N | | 6 | 6 | 5 | 7 |
| Cₘₐₓ | μg/mL | 2.59 ± 0.69 | 6.14 ± 1.88 | 22.6 ± 8.48 | 15.3 ± 4.39 |
| Tₘₐₓ | day | 3.33 ± 1.03 | 4.50 ± 2.35 | 3.99 ± 0.03 | 3.00 ± 2.65 |
| AUCₜₐᵤ^{a} | μg · day/mL | 30.7 ± 7.16 | 72.9 ± 21.3 | 248 ± 109 | 268 ± 79.7 |
| t_{1/2}^{c} | day | 13.4 ± 1.80 | 13.2 ± 2.33 | 10.3 ± 3.52 | 12.6 ± 2.27 |
| Cₘₐₓ/Dose | (μg/mL)/(mg/kg) | 8.63 ± 2.29 | 6.14 ± 1.88 | 7.54 ± 2.83 | 5.11 ± 1.46 |
| AUCₜₐᵤ/Dose | (μg · day/mL)/(mg/kg) | 102 ± 23.9 | 72.9 ± 21.3 | 82.7 ± 36.4 | 89.5 ± 26.6 |
| R_{AC}^{d} | | 2.17 | 2.24 | 2.08 | 1.58 |
| | | [1.81-3.79] | [1.21-2.97] | [1.52-4.49] | [1.05-1.92] |

^{a}AUC_{2wks} for Groups 1, 2 and 3. AUC_{4wks} for Group 4.
^{b}Last dose was administered on Day 43 for EOW Groups 1 to 3 and on Day 56 for E4W Group 4.
^{c}Harmonic mean and pseudo standard deviation.
^{d}R_{AC} = accumulation ratio of AUCₜₐᵤ on Day 43 (EOW) or Day 56 (E4W) to Day 1, as median [min − max].

TABLE 7
Total Variability in ABT-981 Pharmacokinetic Parameters

| | Group 1 | Group 2 | Group 3 | Group 4 |
|---|---|---|---|---|
| Pharmacokinetic | 0.3 mg/kg | 1.0 mg/kg | 3.0 mg/kg | 3.0 mg/kg |
| Parameter (Unit) | EOW | EOW | EOW | E4W |
| | First Dose | | | |
| Cₘₐₓ (μg/mL) | 52 | 41 | 30 | 38 |
| AUCₜₐᵤ (μg · h/mL) | 47 | 38 | 27 | 33 |
| | Final Dose | | | |
| Cₘₐₓ (μg/mL) | 27 | 31 | 38 | 29 |
| AUCₜₐᵤ (μg · h/mL) | 23 | 29 | 44 | 30 |

ABT-981 Cmax and AUC increased in a dose-proportional manner after single doses of 0.3-10 mg/kg IV or 0.3-3 mg/kg SC and multiple doses of 0.3-3 mg/kg SC EOW. Dose-normalized values of Cₘₐₓ and AUCₜₐᵤ were approximately linear between 0.3 and 3 mg/kg EOW following both the first and last dose. Accumulation of AUCₜₐᵤ after the fourth dose was approximately two-fold compared to the first dose during EOW dosing. Estimated relative bioavailability after SC administration was 46%. Following EOW dosing, accumulation in AUCτ was approximately 2-fold. Serum concentrations following SC administration of ABT-981 to subjects with OA of the knee reached maximum levels 5 to 7 days after the first dose, and 3 to 5 days after the final dose. Mean terminal half-life ranged from 10 days to 13 days.

The observed ABT-981 PK profile supports EOW or E4W dosing. PK, immunogenicity and safety profile support further evaluation of ABT-981 as an OA disease modifying agent in Phase 2 studies.

### Example 3

### Analysis of Target Engagement

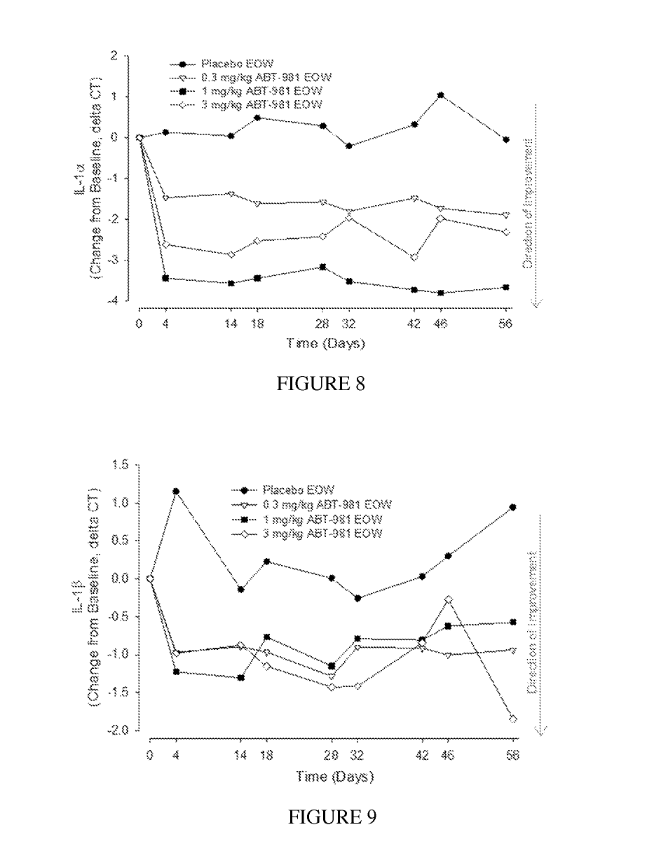

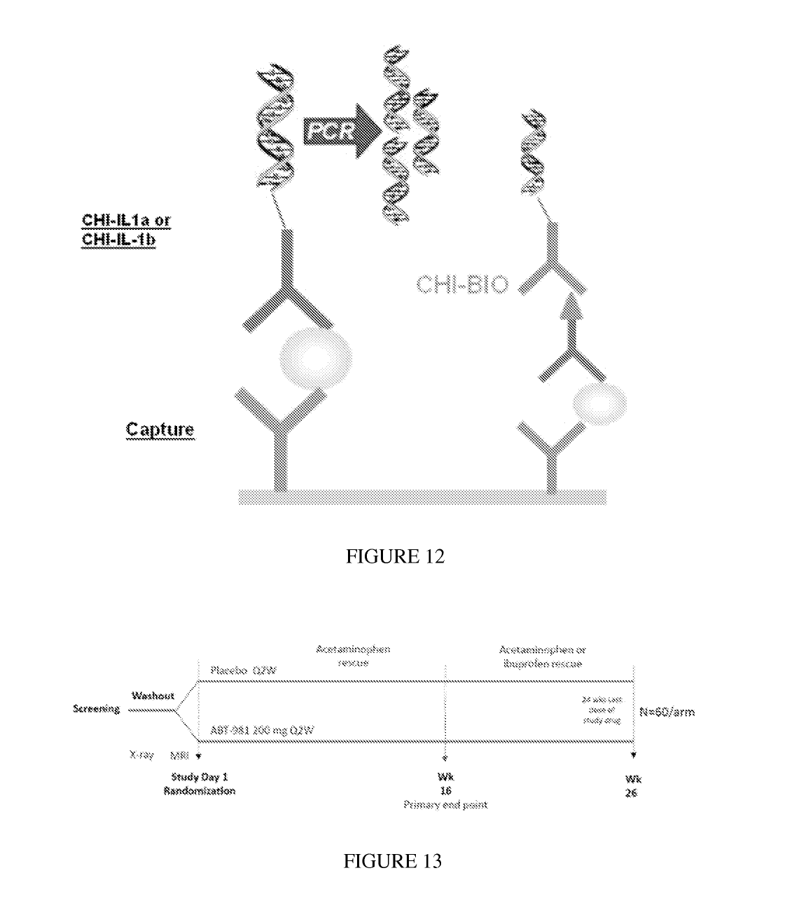

IL-1α and IL-1β levels in serum of subjects administered ABT-981 were measured with a sensitive immuno PCR assay (FIG. 12). The quantification of DNA sequences conjugated to detection antibodies was accomplished using QT-PCR techniques. Antigen levels were expressed in terms of: ΔCt=50-Ct; ΔCt is calculated as Ct, the number of cycles required to reach the threshold fluorescence levels subtracted from 50 (the maximum numbers of cycles). The ΔCt value was then converted to an estimated concentration level by comparing the experimental value to standard values obtained using the same PCR assay. The calculated ΔCt level positively correlated to concentration level in the serum. Data show there was a significant decrease in both IL-1α (P<0.001) and IL-1β (P<0.001) in a dose-dependent manner with ABT-981 compared with placebo (FIG. 8 and FIG. 9).

### Example 4

### Assessment of Biomarkers and Pharmacodynamic Variables

A panel of biomarkers for inflammation and joint degradation were evaluated, including high-sensitivity C-reactive protein (hsCRP), matrix metallopeptidase 9 (MMP-9), vascular endothelial growth factor (VEGF), and MMP degradation products of type I, II, III collagen (C1M, C2M, C3M), C-reactive protein (CRPM), and citrullinated and MMP-degraded vimentin (VICM). Biomarker response for subjects on active drug in each group was compared to the pooled placebo response across groups.

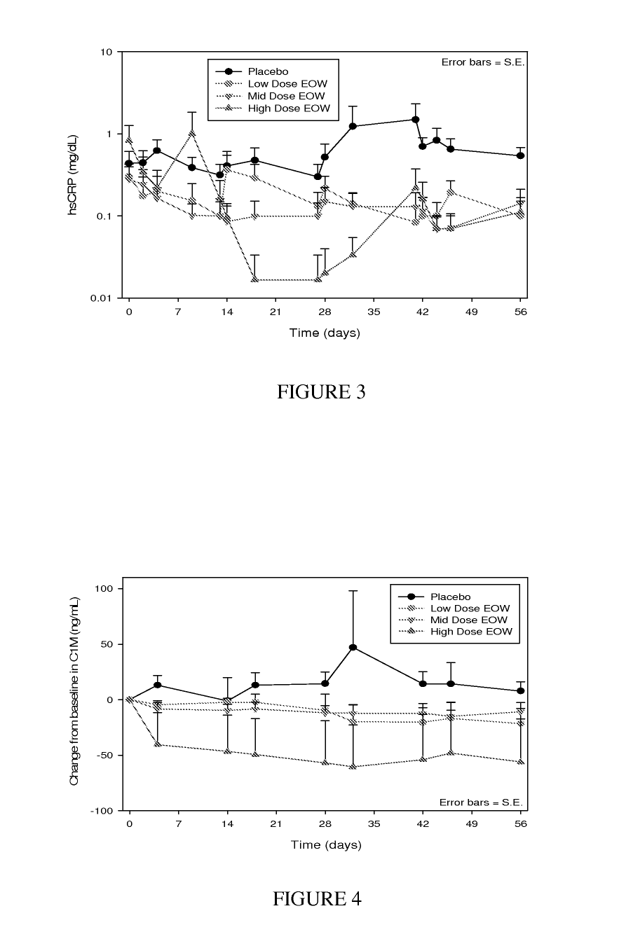

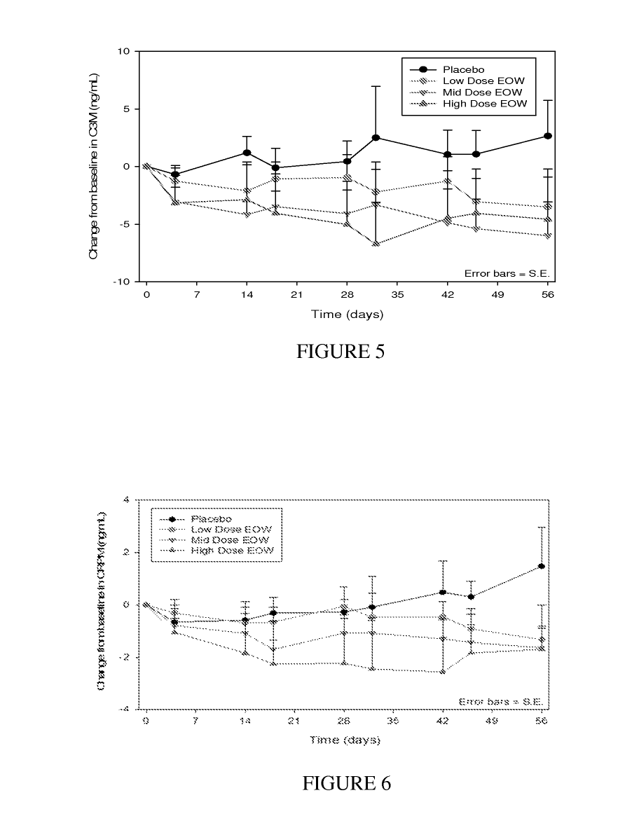

ABT-981 significantly reduced serum absolute neutrophil count and serum levels of hsCRP, C1M, IL1α, and IL-1β. Serum concentrations of C3M and CRPM demonstrated decreasing trends with ABT 981 treatment, but failed to reach statistical significance. The trends among selected biomarkers suggest that ABT-981 is engaging with IL-1α and IL-1β targets, and eliciting an anti-inflammatory response. It was observed that the average level of serum hsCRP in samples from knee OA patients treated with any of the ABT-981 DVD-Ig treatment doses was significantly lower compared to levels in samples from knee OA patients treated with placebo (p-value range from 0.003 to 0.031). See FIG. 3. In addition, FIG. 4 shows that the average serum C1M levels generally decreased in a dose dependent manner in samples obtained from patients administered 0.3, 1 and 3 mg/kg ABT-981 DVD-Ig protein (p=0.062, 0.027 and 0.015 respectively). The C1M levels in samples from ABT-981 DVD-Ig protein treated patients were noticeably lower than samples from patients administered the placebo. Average serum C3M levels were lower for samples from patients administered ABT-981 DVD-Ig protein compared to samples from patients administered the placebo (FIG. 5). Furthermore, the samples from the 1 and 3 mg/kg ABT-981 DVD-Ig treatment groups showed a noticeable decrease in C3M (p=0.062, 0.090 respectively) compared to the samples from the placebo-treated group. Serum CRPM levels were observed to decrease in samples from ABT-981 DVD-Ig treated patients compared to samples from patients treated with the placebo (FIG. 6). In fact the decrease in CRPM levels was statistical difference was starting at about day 33 (p-value range from 0.097 to 0.025).

Data herein show that biomarkers of joint metabolism such as hsCRP are generally elevated in inflammation driven joint destruction diseases. ABT-981 DVD-Ig binding protein, which was designed to simultaneously inhibit IL-1α and IL-1β, significantly reduced systemic inflammation in knee OA patients as evidenced by suppression of serum hsCRP. In addition, it was observed that ABT-981 DVD-Ig protein significantly decreased amount of C1M detected in samples from knee OA patients, which is strong indication that this IL-1α and IL-1β DVD-Ig protein reduced inflammation mediated joint destruction through a decrease of connective tissue turnover. Additionally ABT-981 DVD-Ig protein decreased serum concentrations of C3M and CRPM, which are biomarkers for inflammation mediated tissue destruction and chronic tissue inflammation. It was observed that administration of ABT-981 DVD-Ig protein (ABT-981) to up to 36 mild-to-moderate-knee osteoarthritis patients had an acceptable safety and tolerability profile. The DVD-Ig protein also had a favorable half-life (e.g., 12-14 days) when administered to the patients. Clearly, administration of ABT-981 DVD-Ig provided clinical benefit to this selected population of inflammation driven OA patients.

In the MAD trial (see Example 2), ABT-981 significantly (P<0.001 to P=0.031) reduced serum levels of high-sensitivity C-reactive protein at all 3 doses (hsCRP), matrix metalloproteinase (MMP)-degraded type 1 collagen (C1M), IL-1α (FIG. 8), and IL-1β (FIG. 9). Serum concentrations of MMP-degraded type 3 collagen (C3M) and MMP-degraded CRP (CRPM) demonstrated decreasing trends with ABT-981 treatment but did not reach statistical significance (P=0.054-0.073). These trends suggest that ABT-981 engaged with IL-1α and IL-1β targets and elicited an anti-inflammatory response in patients with knee OA.

In the MAD trial, absolute neutrophil count (ANC) decreased dose-dependently with ABT-981 dosing, starting at 48 hours and reaching nadir by 14 days, with lowest ANC values (2.1-2.3/mm³) observed with 3 mg/kg. Laboratory data from the study suggest a dose relationship with ABT-981 administration and declines in absolute neutrophil count (ANC). Although the 3 mg/kg ABT-981 E4W dose group had a lower baseline ANC than 3 mg/kg ABT-981 EOW dose group, the average maximum decline from baseline in ANC appeared similar for both groups at approximately 30%. The declines in neutrophil count were evident starting around 48-72 hours after initial dosing and reached their nadir in the first 2 weeks of dosing in the study. Consistent with the decline in neutrophil count, a modest decline in white blood cell count was also noted in the 3 mg/kg dose groups. There were no other clinically significant values reported as related to ABT-981 administration for hematology, serum chemistry, urinalysis, vital signs or ECGs. No dose limiting toxicities were observed.

### Example 5

### Phase 2 Study of ABT-981 for Subjects with Knee Osteoarthritis

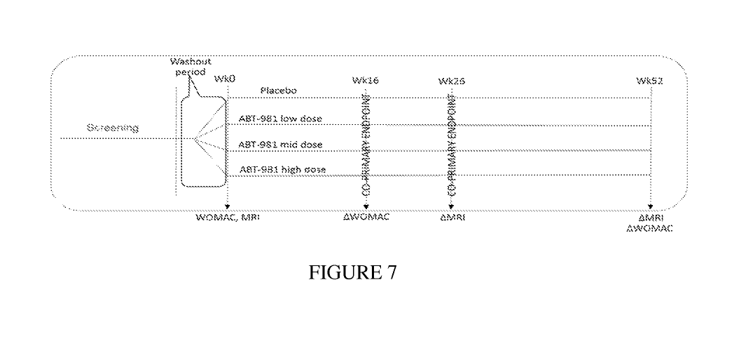

A 52 week study is to be performed to evaluate effects of administering ABT-981 to patients with symptomatic knee osteoarthritis (for study design see FIG. 7). The study will involve a multicenter, randomized, double blind, parallel group, placebo controlled Phase 2a trial to evaluate the safety, tolerability, efficacy, pharmacokinetic and pharmacodynamics effects of ABT-981 in 320 patients with symptomatic, radiographic and inflammatory knee osteoarthritis.

Patients must satisfy certain criteria to be eligible for the study: (1) radiographic evidence of knee osteoarthritis in the medial compartment of the index knee with Kellgren-Lawrence Grade 2 or 3 (with minimum 2 mm joint space width) during Screening as evaluated by a qualified central imaging reader. Prior radiographs taken no more than 3 months before Study Day 1 with Synaflexer™ will be submitted for centralized eligibility reading; (2) The intensity of index knee pain for the patients will be between 4 and 8, inclusive, using the Numeric Rating Scale-11 (NRS-11) at the initial Screening Visit and Study Day 1; (3) An eligible patient will have one or more clinical signs and symptoms of active inflammation in the index knee (localized pain, joint stiffness, swelling and effusion) during Screening and Study Day 1; (4) Eligible patient must also have presence of synovitis in the index knee confirmed by ultrasound during Screening; (5) Eligible patient will have discontinued analgesics, non-steroidal anti-inflammatory drugs and nutraceuticals (e.g., glucosamine, chondroitin sulfate, shark cartilage, diacerein, and soy extract) at least 7 days prior to first dose of study drug until after Week 26 MRI visit. Both males and females will be eligible for the study and the minimum age for the study will be set at 35 years and the maximum age will be set at 74 years.

The study will also have criteria for exclusion of patients including: (1) history of allergic reaction or significant sensitivity to any constituents of the study drug, history of anaphylactic reaction to any agent (e.g., food products or bee stings) or history of a major reaction to any IgG-containing product; (2) significant trauma or surgery to the index knee within the last year or arthroscopy of the index knee within 6 months of screening; (3) Kellgren-Lawrence Grade 1 or 4 in the index knee. (4) severe knee mal-alignment, either greater than 2° in varus; or greater than 5° in valgus angulation in the index knee; and (5) diagnosis of one or more of the following: (a) inflammatory arthritis such as rheumatoid arthritis, autoimmune disorder, seronegative spondyloarthropathy, gout, or pseudogout (defined as acute episodic attacks of swollen, painful joints in a patient with X-Ray chondrocalcinosis or CPPD crystals); and/or (b) other chronic painful syndromes (such as Paget's disease and fibromyalgia) and clinically significant non-articular musculoskeletal pain that could interfere with assessment of pain at the index knee.

Subjects will be screened and will have a washout period in which treatments/medications that subjects are using entering the study are discontinued. At week zero an affected knee of each subject is analyzed by Western Ontario and McMaster Universities (WOMAC) and magnetic resonance imaging. Subjects are then administered different doses of ABT-981. The doses may be administered once or a plurality of times over a period of time. For example, a subject could be administered a dose (e.g., 25 mg, 100 mg and 200 mg) every other week (EOW). The dose amounts and regimen listed herein are exemplary, and are not meant to limit the amount/dose that can be administered or the regimen used by a physician during this study. Control subjects are administered vehicle only, viz., no ABT-981.

The study will be performed in multiple U.S. states and countries and the primary/secondary outcomes analyzed include: changes in Pain Score of the index knee evaluated using Western Ontario and McMaster Universities Osteoarthritis Index (WOMAC) from day 1 to week 16; change in synovitis/effusion volume of the index knee using quantitative and semi-quantitative Magnetic Resonance Imaging (MRI) measurements from day 1 to week 52; change in bone marrow lesions (BML) of the index knee MRI using Whole-Organ Magnetic imaging Scoring (WORMS) from day 1 to week 52; change in index knee resting pain using the Intermittent and Constant Osteoarthritis Pain (ICOAP) score from day 1 to week 52; change in three types of pain intensity measures of the index knee using the 11-point NRS scale (NRS-11); and change in Patient Global Assessment of Arthritis using Patient Global Assessment of Arthritis form from day 1 to week 52.

For the study, a co-primary endpoint is analyzed at Week 16 and involves determining change from baseline in WOMAC pain. Another co-primary endpoint is analyzed at Week 26 and involves using MRI to determine synovitis and/or loss of knee cartilage volume. Other primary endpoints and/or secondary endpoints may also be analyzed. At Week 52 subjects having received ABT-981 will be analyzed for change from baseline in WOMAC pain and loss of knee cartilage volume measured by MRI compared to control subjects.

### Example 6

### Phase 2 Study of ABT-981 for Subjects with Erosive Hand Osteoarthritis

Erosive hand osteoarthritis (eHOA) is a painful, debilitating arthritis in the hands. It often occurs in peri- and postmenopausal females and can start as early as late 30's and early 40's. Effective drug treatment or surgical options are not currently available to erosive hand OA patients. Compared to generalized OA, erosive hand OA is a more inflammation-driven and a more rapidly progressing disease. Additionally, erosive hand OA is typically polyarticular in nature. Pharmacodynamic effects and/or efficacy of an anti-inflammatory treatment such as ABT-981 may be more readily detectable in the multiple joints implicated in the disease. Hence, erosive hand OA is a compelling model for establishing proof-of-concept (POC) with an anti-inflammatory drug, i.e., a disease modifying osteoarthritis drug (DMAOD) such as ABT-981.

A Phase 2a randomized, double-blind, placebo controlled, proof-of-concept study is performed to evaluate the safety and efficacy of ABT-981 in patients with eHOA (see FIG. 13). Subjects who meet all the inclusion criteria including a diagnosis of hand OA and none of the exclusion criteria during screening will be evaluated with high quality radiographs of both hands to determine eligibility for the study. Eligible subjects will be required to discontinue all medications taken for OA or OA pain. Following the Washout Period and prior to the Day 1 Visit, subjects undergo a magnetic resonance imaging (MRI) of the index hand. The index hand is defined as the hand with the most active disease as determined by the number of tender and swollen joints. About 120 subjects will be randomized in an equal ratio to one of two treatment groups: ABT-981 200 mg subcutaneous injection every two weeks for 24 weeks; or Placebo subcutaneous injection every two weeks for 24 weeks.

The primary efficacy endpoint is the change of pain from Baseline to 16 weeks as assessed by the Australian/Canadian Osteoarthritis Hand Index (AUSCAN NR3.1) pain subdomain score. Secondary Efficacy Endpoints include: the change of total AUSCAN score and individual subdomain (pain, physical function and stiffness); the change of subject index hand resting pain; and change of Patient Global Assessment. Pharmacokinetic and immunogenicity are evaluated during the study. Pharmacodynamic assessments include grip strength, Hand Osteoarthritis Magnetic Resonance Imaging Scoring System (HOAMRIS) score and biomarkers. Safety is monitored throughout the study based on assessments of adverse events (AEs), physical examinations, vital signs, and laboratory values.

### INCORPORATION BY REFERENCE

The contents of all cited references (including literature references, patents, patent applications, and websites) that may be cited throughout this application are hereby expressly incorporated by reference in their entirety, as are the references cited therein. The practice of the present invention will employ, unless otherwise indicated, conventional techniques of pharmaceutical science, immunology, molecular biology, and cell biology, which are well known in the art.

### EQUIVALENTS

The invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims rather than by the foregoing description, and all changes that come within the meaning and range of equivalency of the claims are therefore intended to be embraced herein.

1. A method for reducing one or more symptoms of osteoarthritis in an individual comprising administering to the individual
a binding protein that binds both IL-1α and IL-1β, wherein the binding protein is a dual variable domain immunoglobulin (DVD-Ig) binding protein including a variable heavy chain comprising an amino acid sequence selected from SEQ ID NOs: 46, 56, 66, 76, 86, 96, 106, 116, and 126, and including a variable light chain comprising an amino acid sequence selected from SEQ ID NOs: 51, 71, 81, 91, 101, 111, 121, and 131,
whereby one or more symptoms of osteoarthritis is reduced.

2. The method according to claim 1, wherein the osteoarthritis is moderate-to-severe knee osteoarthritis or moderate-to-severe erosive hand osteoarthritis.

3. A method for reducing pain associated with osteoarthritis in an individual, comprising administering to the individual
a binding protein that binds both IL-1α and IL-1β, wherein the binding protein is a DVD-Ig binding protein including a variable heavy chain comprising an amino acid sequence selected from SEQ ID NOs: 46, 56, 66, 76, 86, 96, 106, 116, and 126, and including a variable light chain comprising an amino acid sequence selected from SEQ ID NOs: 51, 71, 81, 91, 101, 111, 121, and 131,
whereby the pain is reduced.

4. The method for reducing pain according to claim 3, wherein the individual suffers from a pain condition selected from the group consisting of allodynia, hyperalgesia, and a combination of allodynia and hyperalgesia.

5. The method according to claim 1, wherein administering to said individual is subcutaneous administration or intravenous administration.

6. The method according to claim 1, wherein the binding protein is administered at a dose of
from about 0.1 mg/kg to about 10 mg/kg;
from about 0.3 mg/kg to about 3 mg/kg;
from about 1 mg/kg to about 3 mg/kg; or
about 3 mg/kg.

7-9. (canceled)

10. The method according to claim 1, wherein the binding protein is administered at a total dose of between about 1-25 mg, about 25-50 mg, about 50-75 mg, about 75-100 mg, about 100-200 mg, about 100-125 mg, about 125-150 mg, about 150-175 mg, about 175-200 mg, about 200-225 mg, about 225-250 mg, about 250-275 mg, about 275-300 mg, 300-325 mg, or about 325-350 mg of the binding protein.

11. (canceled)

12. The method according to claim 1, wherein the binding protein is administered:
in a single dose;
in multiple doses;
every week;
every other week;
every three weeks; or
every four weeks.

13-15. (canceled)

16. The method according to claim 1, wherein reducing the one or more symptoms of osteoarthritis comprises a decrease in one or more biomarker levels selected from the group consisting of high-sensitivity C-reactive protein (hsCRP), MMP degradation product type I (C1M), MMP degradation product type III (C3M) and C-reactive protein (CRPM) is observed in the subject after receiving one or more doses of the DVD-Ig binding protein relative to the one or more biomarker levels in the subject prior to receiving the one or more doses of the DVD-Ig binding protein.

17. The method of claim 1, wherein reducing the one or more symptoms of osteoarthritis comprises a decrease in one or more parameter levels selected from the group consisting of systemic inflammation, chronic tissue inflammation, inflammation-mediated tissue destruction, inflammation-mediated joint destruction, and connective tissue turnover is observed in the subject after receiving one or more doses of the DVD-Ig binding protein relative to the one or more parameter levels in the subject prior to receiving the one or more doses of the DVD-Ig binding protein.

18. The method of claim 1, wherein reducing the one or more symptoms of osteoarthritis comprises a decrease in one or more characteristics selected from the group consisting of pain, joint swelling, joint stiffness, effusion, rate of bone lesion, rate of joint space narrowing, rate of bony deformity formation, rate of bone sclerosis, synovitis, synovial hypertrophy, synovial hyperplasia, angiogenesis, and the presence of osteophytes is observed in the subject after receiving one or more doses of the DVD-Ig binding protein relative to the one or more characteristics in the subject prior to receiving the one or more doses of the DVD-Ig binding protein.

19. The method according to claim 1, wherein reducing the one or more symptoms of osteoarthritis comprises an improvement in one or more metrics selected from the group consisting of Western Ontario and McMaster Universities Arthritis Index (WOMAC), Whole-Organ Magnetic Imaging Score (WORMS), Intermittent and Constant Osteoarthritis Pain (ICOAP) score; 11-point Numeric Rating Score (NRS) score, Physician Global Assessment of Disease Activity, Patient Reported Outcome, a Health Assessment Questionnaire (HAQ-DI), a patient global assessment of disease activity (VAS), measurement or presence of an anti-drug antibody (ADA), tender joint count (TJC), swollen joint count (SJC), patient's assessment of pain, Work Instability Scale for Rheumatoid Arthritis, Short Form Health Survey (SF-36), American College of Rheumatology (ACR); proportion of subjects achieving Low Disease Activity (LDA); Disease Activity Score 28 (DAS28); Clinical Disease Activity Index (CDAI), simple disease activity index (SDAI), Clinical Remission criteria, and the individual's assessment is observed in the subject after receiving one or more doses of the DVD-Ig binding protein relative to the one or more metrics in the subject prior to receiving the one or more doses.

20. The method according to claim 1, wherein the binding prevents degradation or loss of cartilage.

21. A method of reducing one or both of osteoarthritis and pain associated with osteoarthritis in a human subject, the method comprising
administering to the human subject a binding protein that binds both IL-1α and IL-1β,
wherein administering the binding protein is performed using a dose of between about 1 to about 3 mg/kg of weight of the binding protein to weight of the individual, or
wherein administering the binding protein is performed using a dose of between about 100 mg and about 200 mg of the binding protein, and
wherein a decrease in one or more biomarker levels selected from the group consisting of hsCRP, C1M, C3M and C-reactive protein CRPM is observed in the subject after receiving one or more doses of the binding protein that binds both IL-1α and IL-1β relative to the one or more biomarker levels in the subject prior to receiving the one or more doses of the binding protein that binds both IL-1α and IL-1β, to reduce one or both of the osteoarthritis and the pain associated with osteoarthritis.

22. The method of claim 21, wherein the binding protein comprises a DVD-Ig binding protein binding protein including a variable heavy chain comprising an amino acid sequence selected from SEQ ID NOs: 46, 56, 66, 76, 86, 96, 106, 116, and 126, and including a variable light chain comprising an amino acid sequence selected from SEQ ID NOs: 51, 71, 81, 91, 101, 111, 121, and 131.

23. The method according to claim 21, wherein the osteoarthritis is moderate-to-severe knee osteoarthritis or moderate-to-severe erosive hand osteoarthritis.

24. The method according to claim 21, wherein administering to said individual is by subcutaneous administration or intravenous administration.

25. The method according to claim 21, wherein the binding protein is administered:
at a dose of between about 1 and about 3 mg/kg;
at a dose of about 3 mg/kg; or
at a total dose of between about 100 mg and about 200 mg.

26-27. (canceled)

28. The method according to claim 21, wherein the binding protein is administered:
in a single dose;
every other week; or
every four weeks.

29. (canceled)

30. A method of decreasing one or more biomarker levels associated with osteoarthritis in a subject comprising
administering to the subject a DVD-Ig binding protein binding protein including a variable heavy chain comprising an amino acid sequence selected from SEQ ID NOs: 46, 56, 66, 76, 86, 96, 106, 116, and 126, and including a variable light chain comprising an amino acid sequence selected from SEQ ID NOs: 51, 71, 81, 91, 101, 111, 121, and 131,
wherein administering the binding protein is performed using a dose of between about 1 to about 3 mg/kg of weight of the binding protein to weight of the individual, or
wherein administering the binding protein is performed using a dose of between about 100 mg and about 200 mg of the binding protein, and
wherein a decrease in one or more biomarker levels selected from the group consisting of hsCRP, C1M, C3M and C-reactive protein CRPM is observed in the subject after administration of the DVD-Ig binding protein relative to the one or more biomarker levels in the subject prior to administration of the DVD-Ig binding protein, to decrease one or more biomarker levels associated with osteoarthritis.

31. The method according to claim 30, wherein the osteoarthritis is moderate-to-severe knee osteoarthritis or moderate-to-severe erosive hand osteoarthritis.

32. The method according claim 30, wherein administering to said individual is by subcutaneous administration or intravenous administration.

33. The method according to claim 30, wherein the binding protein is administered:
at a dose of between about 1 and about 3 mg/kg;
at a dose of about 3 mg/kg; or
at a total dose of between about 100 mg and about 200 mg;
in a single dose;
every other week; or
every four weeks.

34-37. (canceled)

38. A method for treating osteoarthritis in a human subject comprising the step of administering to the human subject a binding protein that binds both IL-1α and IL-1β, wherein the binding protein is a DVD-Ig binding protein including a variable heavy chain comprising an amino acid sequence selected from SEQ ID NOs: 46, 56, 66, 76, 86, 96, 106, 116, and 126, and including a variable light chain comprising an amino acid sequence selected from SEQ ID NOs: 51, 71, 81, 91, 101, 111, 121, and 131, in a dose to achieve:
(a) an area under the curve (AUC) of between about 5 and about 300 μg×day/mL;
(b) a serum or plasma half-life (T½) of at least about 8 days;
(c) a time point to maximum observed serum concentration (Tmax) of between about 2 days and about 8 days; and/or
(d) a maximum observed serum concentration (Cmax) of between about 0.5 and about 25 μg/mL,
following administration of the DVD-Ig binding protein to the human subject.

39. The method of claim 38, wherein the AUC is between about 12 and about 280 μg×day/mL, the T½ is at least about 10 days, the Tmax is between about 2.5 days and about 7 days, and/or the Cmax is between about 0.1 and about 23 μg/mL.

40. The method of claim 38, wherein the AUC is at least about 30 μg×day/mL, the T½ is at least about 10 days, the Tmax is less than about 7 days, and/or the Cmax is at least about 2.5 μg/mL.

41. A method for treating pain associated with osteoarthritis in a human subject, wherein the pain is associated with osteoarthritis, the method comprising the step of administering to the human subject a binding protein that binds both IL-1α and IL-1β, wherein the binding protein is a DVD-Ig binding protein including a variable heavy chain comprising an amino acid sequence selected from SEQ ID NOs: 46, 56, 66, 76, 86, 96, 106, 116, and 126, and including a variable light chain comprising an amino acid sequence selected from SEQ ID NOs: 51, 71, 81, 91, 101, 111, 121, and 131, in a dose to achieve:
(a) an AUC of between about 5 and about 300 μg×day/mL;
(b) a T½ of at least about 8 days;
(c) a Tmax of between about 2 days and about 8 days; and/or
(d) a Cmax of between about 0.5 and about 25 μg/mL,
following administration of the DVD-Ig binding protein to the human subject.

42. The method of claim 41, wherein the AUC is between about 12 and about 280 μg×day/mL, the T½ is at least about 10 days, the Tmax is between about 2.5 days and about 7 days, and/or the Cmax is between about 0.1 and about 23 μg/mL.

43. The method of claim 41, wherein the AUC is at least about 30 μg×day/mL, the T½ is at least about 10 days, the Tmax is less than about 7 days, and/or the Cmax is at least about 2.5 μg/mL.
